(19) United States
(12) Patent Application Publication
(10) Pub. No.: US 2004/0023225 A1
(43) Pub. Date: Feb. 5, 2004

(54) Methods and compositions for identifying risk factors for abnormal lipid levels and the diseases and disorders associated therewith

(75) Inventor: Jeanette McCarthy, San Diego, CA (US)

Correspondence Address: MILLENNIUM PHARMACEUTICALS, INC., 75 SIDNEY STREET, CAMBRIDGE, MA, 02139, US

(73) Assignee: Vitivity, Inc.

(21) Appl. No.: 10/212,848

(22) Filed: Aug. 5, 2002

(51) Int. Cl. C12Q 1/68

(52) U.S. Cl. 435/6

(57) ABSTRACT

The present invention is based, at least in part, on the identification of associations between polymorphic regions of the CD36L1 gene and specific diseases or disorders, e.g., abnormal lipid levels, e.g., abnormally high TG level or an abnormally high TG:HDL-C ratio, or diseases or disorders associated with abnormal lipid levels, e.g., vascular or metabolic diseases or disorders. The invention also provides methods for identifying specific alleles of polymorphic regions of a CD36L1 gene, methods for determining whether a subject has or is at risk of developing abnormal lipid levels, e.g., high TG level and high TG:HDL-C levels, or a disease or disorder associated therewith, e.g., a vascular disease or disorder or a metabolic disease or disorder, based on detection of one or more polymorphisms within the CD36L1 gene, and kits for performing such methods.

### BACKGROUND OF THE INVENTION

Coronary artery disease (CAD) is a major health risk throughout the industrialized world. Atherosclerosis, the most prevalent of cardiovascular diseases, is the principal cause of myocardial infarction, stroke, and gangrene of the extremities, and thereby the principle cause of death in the United States. Myocardial infarction (MI), e.g., heart attack, results when the heart is damaged due to reduced blood flow to the heart caused by the build-up of plaque in the coronary arteries.

Decreased HDL-C and elevated triglyceride (TG) levels are well-known risk factors for the development of CAD and atherosclerosis (NIH Consensus Development Panel on TG, High-Density Lipoprotein and Coronary Heart Disease JAMA 1993:269:505-10). Although plasma HDL-C and TG levels are highly correlated, there is evidence that they may be independent risk factors and that simultaneous evaluation of both may more accurately predict risk of coronary disease. Among patients with low HDL-C, risk of CAD more than doubled in those with concurrent high TG compared to those with low TG in both the PROCAM(Assmann, G. and Schulte, H. (1992) Am. J. Cardiol. 70(7):733-7) and Helsinki Heart studies (Manninen, V., et al. (1992) 85(1):37-45). The ratio of TG:HDL-C has also been shown to correlate with LDL particle size (Dobiasove, M. and Frohlich, J. (2001) Clin. Biochem. 34(7):584-8) itself atherogenic, may therefore be a powerful predictor of future MI (Gaziano, et al. (1997) Circulation 85(1):37-45).

Plasma lipid concentrations are complex traits having both environmental and genetic determinants. About half of the variation in HDL-C and TG may be genetic (Perusse, et al. (1997) Arterioscler Thromb Vasc Biol 17(11):3263-9; Rice, T. et al. (1991) Hum Hered 41(2):107-21; Austin, M. A. et al. (1987) Am J. Epidemiol 125(2):308-18) with the same genes possibly accounting for the correlated traits of LDL particle size, TG and HDL-C (Edwards, et al. (1999) Arterioscler Thromb Biol 19(10):2456-64). A number of variants in candidate genes have been implicated in the regulation of plasma lipid levels (Breslow, J. L. (2000) Annu Rev Genet 34:233-254). In addition, genome scans have identified chromosomal regions with suggestive linkage to the TG:HDL-C ratio and other lipid parameters (Shearman, et al. (2000) Hum Mol Genet 9(9):1315-20; Klos, et al. (2001) Arterioscler Thromb Vasc Biol 21(6):971-8). Nonetheless, much of the genetic variability in HDL-C and TG levels remains unexplained.

The scavenger receptor class B type 1, CD36L1 (also referred to as SR-B1 and CLA-1) is a key component in the reverse cholesterol transport pathway where it binds HDL-C with high affinity and is involved in the selective transfer of lipids from HDL-C (Acton, et al. (1996) Science 271:518-520; Rigotti, A. et al. (1997) Proc Natl Acad Sci USA 94:12160-5). It is expressed primarily in liver and nonplacental steroidogenic tissues and mediates selective cholesterol uptake by a mechanism distinct from the classic low-density lipoprotein cholesterol (LDL-C) receptor pathway (Varban, et al. (1998) Proc Acad Nat Acad Sci 95:4619-24). CD36L1 receptor exists in two differentially spliced forms (Calvo and Vega (1993) J. Biol. Chem. 268:18929). The predominant form of human CD36L1 is a protein of 509 amino acids. The shorter form of CD36L1 has 409 amino acids, and is lacking the 100 amino acids located 42 amino acids downstream of the initiation codon (Calvo and Vega, supra). The nucleotide sequence of a cDNA encoding human CD36L1 is disclosed in Calvo and Vega, supra, and the nucleotide sequence of a cDNA encoding hamster CD36L1 is disclosed in Acton et al. (1994) J. Biol. Chem. 269:21003 and in PCT Application WO 96/00288.

Previous studies (Acton, et al. (1999) Arteriolscler Thromb Vasc Biol 19:1734-43; McCarthy, et al. (2001) Am J Hum Genet 69(4):383 (abstract), U.S. Pat. Nos. 6,030,778, and 6,228,581, and U.S. patent application Ser. No. 09/779,152, the entire contents of which are incorporated herein by reference) have found single nucleotide polymorphisms (SNPs) in CD36L1 associated with plasma lipids. In one study, three populations of diabetic kindred showed an association between combinations of SNPs in CD36L1 and HDL-C that appeared to be modified by gender. The most common cause of low HDL-C associated with CAD is hypertriglyceridemia (Genest, et al. (1992) Circulation 85:2025-33). Accordingly, it would be useful to identify associations between CD36L1 single nucleotide polymorphisms (SNPs) and HDL-C, TG and the TG:HDL-C ratio. It would further be desirable to provide prognostic, diagnostic, pharmacogenomic, and therapeutic methods utilizing the identified associations.

### SUMMARY OF THE INVENTION

The present invention is based, at least in part, on the identification of associations between known polymorphic regions of the CD36L1 gene and specific diseases or disorders, e.g., abnormal lipid levels, e.g., abnormally high TG level or an abnormally high TG:HDL-C ratio, or diseases or disorders associated with abnormal lipid levels, e.g., a vascular or metabolic disease or disorder. In particular, the present invention is based, at least in part, on the on the discovery of a significant association between the combination of two known polymorphisms, e.g., the combination of polymorphisms at position 41 of exon 8 (EX8) and position 54 of intron 5 (IVS5), within the CD36L1 gene, TG level, and TG:HDL-C ratio in women with premature, familial CAD (see Table I, below). Decreased HDL-C and elevated TG levels are well-known risk factors for the development of vascular diseases and disorders, e.g., CAD and MI and metabolic diseases or disorders, e.g., diabetes or obesity. Accordingly, SNPs in this gene can be utilized to predict an increased risk for developing a vascular disease or disorder, e.g., CAD or MI, or a metabolic disease or disorder, e.g., diabetes or obesity.

Furthermore, the associations between EX8 and IVS5 and high TG and/or high TG:HDL-C ratios are influenced by gender, indicating an interaction with hormonal status. The association of lipids with CD36L1 variants is modulated by hormonal status (see Example 2). Therefore, these CD36L1 polymorphisms, used in combination with each other, may be useful in predicting the effect of hormone replacement therapy (HRT) on lipid levels in female subjects, e.g., postmenopausal female subjects, and therefore the risk for CAD and/or MI.

The human CD36L1 gene contains 12 coding exons and one non coding exon (exon 13). The structure of the gene and the position of the introns relative to the nucleotide sequence of the exons are shown in FIGS. 1, 2, and 3.

Thus, the invention relates to polymorphic regions and in particular, SNPs identified as described herein, in combination with each other or in combination with other polymorphisms in the CD36L1 gene or in other genes. The invention also relates to the use of these SNPs, and other SNPs in the CD36L1 gene or in other genes, particularly those in linkage disequilibrium with these SNPs, for diagnosis of abnormal lipid levels, e.g., abnormally high TG level or an abnormally high TG:HDL-C ratio, or a disease or disorder associated with abnormal lipid levels, e.g., a vascular or metabolic disease or disorder. The SNPs identified herein may further be used in the development of new treatments for vascular disease based upon comparison of the variant and normal versions of the gene or gene product (e.g., the reference sequence), and development of cell-culture based and animal models for research and treatment of vascular disease. The invention further relates to novel compounds and pharmaceutical compositions for use in the diagnosis and treatment of such disorders. In preferred embodiments, the vascular disease is CAD or MI.

The polymorphisms of the invention may thus be used, or in combination with each other or with polymorphisms in the CD36L1 gene or in other genes, in prognostic, diagnostic, and therapeutic methods. For example, the polymorphisms of the invention can be used to determine whether a subject has, or is, or is not at risk of developing a disease or disorder associated with a specific allelic variant of a CD36L1 polymorphic region, e.g., a disease or disorder associated with aberrant CD36L1 activity, e.g., abnormal lipid levels, e.g., abnormally high TG level or an abnormally high TG:HDL-C ratio, or a disease or disorder associated with abnormal lipid levels, e.g., a vascular or metabolic disease or disorder.

The invention further provides methods for determining at least a portion of a CD36L1 gene. In a preferred embodiment, the method comprises contacting a sample nucleic acid comprising a CD36L1 gene sequence with a probe or primer having a sequence which is complementary to a CD36L1 gene sequence, carrying out a reaction that would amplify and/or detect differences in a region of interest within the CD36L1 gene sequence, and comparing the result of each reaction with that of a reaction with a control (known) CD36L1 gene (e.g., a CD36L1 gene from a human not afflicted with a vascular disease or disorder e.g., CAD, MI, or another disease associated with an aberrant CD36L1 activity) so as to determine the molecular structure of the CD36L1 gene sequence in the sample nucleic acid. The method of the invention can be used for example in determining the molecular structure of at least a portion of an exon, an intron, a 5′ upstream regulatory element, or the 3′ untranslated region In another preferred embodiment, the method comprises determining the nucleotide content of at least a portion of a CD36L1 gene, such as by sequence analysis. In yet another embodiment, determining the molecular structure of at least a portion of a CD36L1 gene is carried out by single-stranded conformation polymorphism (SSCP). In yet another embodiment, the method is an oligonucleotide ligation assay (OLA). Other methods within the scope of the invention for determining the molecular structure of at least a portion of a CD36L1 gene include hybridization of allele-specific oligonucleotides, sequence specific amplification, primer specific extension, and denaturing high performance liquid chromatography (DHPLC). In at least some of the methods of the invention, the probe or primer is allele specific. Preferred probes or primers are single stranded nucleic acids, which optionally are labeled.

The methods of the invention can be used for determining the identity of a nucleotide or amino acid residue within a polymorphic region of a human CD36L1 gene present in a subject. For example, the methods of the invention can be useful for determining whether a subject has, or is or is not at risk of developing abnormal lipid levels, e.g., abnormally high TG level or an abnormally high TG:HDL-C ratio, or a disease or disorder associated with abnormal lipid levels, e.g., a vascular or metabolic disease or disorder.

In one embodiment, the disease or condition is characterized by an aberrant CD36L1 activity, such as aberrant CD36L1 protein level, which can result from aberrant expression of a CD36L1 gene. Accordingly, the invention provides methods for predicting abnormal lipid levels, e.g., abnormally high TG level or an abnormally high TG:HDL-C ratio. The invention also provides a method of identifying subjects which are at increased risk of developing CAD and/or MI, wherein the method comprises the steps of i) identifying in DNA from a subject at least one sequence polymorphism, as compared with the reference CD36L1 gene sequence which comprises SEQ ID NO:1, in a CD36L1 gene sequence; and ii) identifying the subject based on the identified polymorphism.

In another embodiment, the invention provides a kit for amplifying and/or for determining the molecular structure of at least a portion of a CD36L1 gene, comprising a probe or primer capable of hybridizing to a CD36L1 gene and instructions for use. In a preferred embodiment, determining the molecular structure of a region of a CD36L1 gene comprises determining the identity of the allelic variant of the polymorphic region. Determining the molecular structure of at least a portion of a CD36L1 gene can comprise determining the identity of at least one nucleotide or determining the nucleotide composition, e.g., the nucleotide sequence a CD36L1 gene.

A kit of the invention can be used, e.g., for determining whether a subject is or is not at risk of developing a disease associated with a specific allelic variant of a polymorphic region of a CD36L1 gene. In a preferred embodiment, the invention provides a kit for determining whether a subject is or is not at risk of developing abnormal lipid levels, e.g., abnormally high TG level or an abnormally high TG:HDL-C ratio, or a disease or disorder associated with abnormal lipid levels, e.g., a vascular or metabolic disease or disorder. Vascular diseases or disorders include diseases such as, for example, atherosclerosis, CAD, MI, ischemia, stroke, peripheral vascular diseases, venous thromboembolism and pulmonary embolism. The kit of the invention can also be used in selecting the appropriate clinical course of treatment for a subject. Thus, determining the allelic variants of CD36L1 polymorphic regions of a subject can be useful in predicting how a subject will respond to a specific drug, e.g., a drug for treating a disease or disorder associated with aberrant CD36L1, e.g., a vascular or metabolic disease or disorder.

In a further embodiment, the invention provides a method for treating a subject having a disease or condition associated with a specific allelic variant of a polymorphic region of a CD36L1 gene. In one embodiment, the method comprises the steps of (a) determining the identity of the allelic variant; and (b) administering to the subject a clinical course of therapy that compensates for the effect of the specific allelic variant e.g., treatment with medications, lifestyle changes, and any combination thereof. In one embodiment, the clinical course of therapy is administration of an agent or modulator which modulates, e.g., agonizes or antagonizes, CD36L1 nucleic acid expression or CD36L1 protein levels. In a preferred embodiment, the modulator is selected from the group consisting of a nucleic acid, a ribozyme, an antisense CD36L1 nucleic acid molecule, a CD36L1 protein or polypeptide, an antibody, a peptidomimetic, or a small molecule.

In a preferred embodiment, the specific allelic variant is a mutation. The mutation can be located, e.g., in a 5′ upstream regulatory element, a 3′ regulatory element, an intron, or an exon of the gene. Thus, for example, in a subject that is heterozygous (CT) at IVS5 and heterozygous (CT) at EX8, or the complements thereof, a subject that is homozygous (TT) at IVS5 and heterozygous (CT) at EX8, or the complements thereof, vascular disorders such as CAD or MI, can be treated, prevented, or ameliorated by administering to the subject a particular clinical course of treatment sufficient to treat, prevent, or ameliorate the vascular disease or disorder.

Additionally, the invention provides a method of identifying a subject who is susceptible to abnormal lipid levels, e.g., abnormally high TG level or an abnormally high TG:HDL-C ratio, which method comprises the steps of i) providing a nucleic acid sample from a subject; and ii) detecting in the nucleic acid sample one or more CD36L1 gene polymorphisms, that correlate with the vascular disorder with a P value less than or equal to 0.05, the existence of the polymorphism being indicative of susceptibility to abnormal lipid levels, e.g., abnormally high TG level or an abnormally high TG:HDL-C ratio.

In another aspect, the invention provides methods for predicting the effect of hormone replacement therapy (HRT) on the TG level or TG:HDL-C ratio in a female subject, e.g., a postmenopausal female subject, comprising identifying one or more allelic variants of the CD36L1 gene which are associated with abnormally high TG level or TG:HDL-C ratio in females, thereby predicting the effect of hormone replacement therapy on the TG level or TG:HDL-C ratio in the subject. In one embodiment, the presence of CT at IVS5 and CT at EX8, or the complements thereof, or TT at IVS5 and CT at EX8, or the complements thereof, indicates the effect of hormone replacement therapy in a subject to be an increase in TG level or TG:HDL-C ratio.

The invention further provides forensic methods based on detection of polymorphisms within the CD36L1 gene

Other features and advantages of the invention will be apparent from the following detailed description and claims.

### BRIEF DESCRIPTION OF THE FIGURES

FIG. 1 is a schematic depiction of the chromosomal structure of the human CD36L1 gene indicating the introns (1 through 12) and exons (I-XIII). Black boxes represent coding exons (exons I-XII) and the white box represents the non-coding exon (exon XIII) and the nucleotides in the newly identified alleles are indicated.

FIG. 2A-G represents the nucleotide sequence of the exons (underlined sequence) of the human CD36L1 gene, portions of the introns which are adjacent to the exons, and 3′ end of the promoter sequence (SEQ ID Nos. 5-40). The putative 5′ end of the cDNA, as predicted by GRAIL, is indicated in italics. The TATA-like box is indicated in italics and is boxed. Bold sequences correspond to the nucleotide sequence or the complement of the nucleotide sequence of preferred primers for amplifying each of the exons or a promoter region. The nucleotide polymorphisms in exons 1, 3, and 8 and introns 5 and 10 are boxed.

FIG. 3A-B shows the nucleotide sequence of the full length human CD36L1 cDNA (SEQ ID NO: 1) and the position of introns 1-12 relative to the nucleotide sequence of the exons. The nucleotide polymorphisms in exons 1, 3, and 8 are boxed.

FIG. 4 depicts the proportion of male patients from the GeneQuest study by HDL-C and TG category.

FIG. 5 depicts the proportion of female patients from the GeneQuest study by HDL-C and TG category.

### DETAILED DESCRIPTION OF THE INVENTION

The present invention is based, at least in part, on the identification of associations between a combination of known polymorphic regions of the CD36L1 gene and specific diseases or conditions in women, e.g., abnormal TG levels and abnormal TG:HDL-C ratio. Decreased HDL-C and elevated TG levels are well-known risk factors for the development of vascular diseases or disorders, e.g., CAD and MI. Decreased HDL-C and elevated TG levels are also hallmark dyslipidemias of metabolic diseases or disorders, e.g., diabetes and obesity. Therefore, TG levels and the TG:HDL-C ratio represent important risk factors for vascular diseases or disorders and metabolic diseases or disorders. Thus, the invention relates to the use of specific polymorphic regions and in particular, the SNPs described herein, as well as to the use of these SNPs, and others in the CD36L1 gene, particularly those nearby in linkage disequilibrium with these SNPs, for predicting the risk of developing abnormal lipid levels, e.g., abnormally high TG level or an abnormally high TG:HDL-C ratio, or a disease or disorder associated with abnormal lipid levels, e.g., a vascular or metabolic disease or disorder. Diseases or disorders associated with abnormal TG levels or abnormal TG:HDL-C ratio include vascular diseases or disorders, e.g., CAD or MI, or metabolic diseases or disorders, e.g., diabetes and obesity.

The present invention is based, at least in part, on the on the discovery of significant associations between two common, known polymorphisms, used in combination, e.g., the combination of polymorphisms at position 41 of exon 8 (EX8) and position 54 of intron 5 (IVS5), within the CD36L1 gene, TG level, and TG:HDL-C ratio in women with premature, familial CAD.

Furthermore, the associations between EX8 and IVS5 and high TG and/or high TG:HDL-C ratios are influenced by gender, indicating an interaction with hormonal status. The association of lipids with CD36L1 variants is modulated by hormonal status, as shown in Example 2. Therefore, these CD36L1 polymorphisms, used in combination with each other, may be useful in predicting the effect of hormone replacement therapy (HRT) on lipid levels in female subjects, e.g., postmenopausal female subjects, and therefore the risk for CAD and/or MI.

The polymorphisms described herein may thus be used together, or in combination with other polymorphisms associated with abnormal lipid levels or diseases or disorders associated therewith, e.g., vascular or metabolic diseases or disorders, or in linkage disequilibrium therewith, in prognostic, diagnostic, and therapeutic methods. For example, the polymorphisms described herein can be used to determine whether a subject is or is not at risk of developing a disease or disorder associated with a specific allelic variant of a CD36L1 polymorphic region, e.g., abnormal lipid levels, e.g., abnormally high TG level or an abnormally high TG:HDL-C ratio, or a disease or disorder associated with abnormal lipid levels, e.g., a vascular or metabolic disease or disorder.

Cases which were used to identify associations between TG level and TG:HDL-C level were drawn from the GeneQuest study, a collection of nuclear families consisting of affected sibling pairs and living parents ascertained for premature CAD at fifteen medical centers in the United States. Each proband was required to have developed CAD by age 45 if male, or age 50 if female, as manifest by either a myocardial infarction, surgical or percutaneous coronary revascularization, or a coronary angiogram with evidence of at least a 70% stenosis in a major epicardial artery. At least one living sibling also had to fulfill these qualifying criteria for the family to be enrolled. Probands were identified through patient registries as well as new admissions.

Six previously identified SNPs in the CD36L1 gene were evaluated: a missense (G→A) at nucleotide position 146 in exon 1 resulting in a change from glycine to serine at amino acid 2 in exon 1 (EX1; alleles EX1G and EX1A), a missense (G→A) at nucleotide position 119 in exon 3 resulting in a change from valine to isoleucine at amino acid 135 in exon 3 (EX3; alleles EX3G and EX3A), an intronic SNP (C→T) at nucleotide position 54 of intron 5 (IVS5; alleles IVS5C and IVS5T), a silent mutation (C→T) at amino acid 301 in exon 7 (EX7; alleles EX7C and EX7T), a silent mutation (C→T) at nucleotide position 41, amino acid 350 in exon 8 (EX8; alleles EX8C and EX8T), and an intronic SNP (C→G) 41bp upstream from the start of exon 11 (IVS10; alleles IVS10C and IVS10G).

Combined genotypes for a silent variant in the EX8 SNP and the IVS5 SNP were strongly associated with TG level (p=0.001), and the ratio of TG:HDL-C (p=0.0003) (see Table I, below). These two variants accounted for nearly a quarter of the variability in the ratio of TG:HDL-C in women in the study. The same associations were not found in men, suggesting that the genetic basis of dyslipidemias and their attendant liability for premature coronary disease may differ for men and women.

TABLE I
Mean levels (±SD) of HDL-C, TG and TG:HDL-C ratio by combined
genotypes at IVS5 and EX8 for women.

| IVS5 | EX8 | N | Mean HDL-C | Mean TG | Mean TG:HDL-C |
|---|---|---|---|---|---|
| CT | CT | 3 | 25.0 ± 10.4 | 778.0 ± 570.1 | 38.4 ± 31.2 |
| CT | TT | 1 | 36.0 | 797.0 | 22.1 |
| CC | CC | 20 | 42.4 ± 11.1 | 246.3 ± 207.8 | 6.7 ± 6.1 |
| CC | CT | 44 | 43.0 ± 11.7 | 212.9 ± 139.7 | 5.7 ± 4.8 |
| CC | TT | 22 | 41.1 ± 10.5 | 175.0 ± 74.8 | 4.5 ± 2.1 |
| CT | CC | 10 | 48.3 ± 11.8 | 178.8 ± 110.6 | 3.9 ± 2.6 |
| | | | P = .07 | P = .001 | P = .0003 |

P-values for TG and TG:HDL ratio are for log-transformed data.

Dyslipidemia is a common and heterogeneous manifestation of premature, familial CAD, as well as a risk factor for metabolic diseases or disorders. Among patients from the GeneQuest study, nearly half of all women and two-thirds of men had low HDL-C, often but not always accompanied by elevated TG levels. Combined low HDL-C and elevated TG was found in one quarter of women and about 40% of men. By examining a population enriched for lipid abnormalities, a specific combination of genotypes (IVS5 CT and EX8 CT) that was associated with extremely low HDL-C and extremely high TG in women was identified. Women heterozygous for both polymorphisms had mean HDL-C=25 mg/dl, mean TG=778 mg/dl and TG:HDL-C=38.1 (see Table I). This combination is relatively common (>10%) in men, but seen less frequently in women (˜3%) from the population investigated, suggesting a selective disadvantage for female carriers.

The association of CD36L1 polymorphisms with low HDL-C remained significant after controlling for NIDDM status and BMI, suggesting a lack of confounding by these variables. Other variables known to be associated with serum lipids such as alcohol, smoking and medication use were not included due to the retrospective nature of the study.

The IVS5 and EX8 SNPs are in linkage disequilibrium. The term “linkage disequilibrium,” also referred to herein as “LD,” refers to a greater than random association between specific alleles at two marker loci within a particular population. In general, linkage disequilibrium decreases with an increase in physical distance. If linkage disequilibrium exists between two markers, or SNPs, then the genotypic information at one marker, or SNP, can be used to make probabilistic predictions about the genotype of the second marker.

Because the IVS5 and EX8 SNPs are in linkage disequilibrium, their combined effect on lipid levels may reflect an underlying haplotype(s), or segment of DNA defined by several variants inherited together as a unit, that is the basis of the observed association. Haplotypes can be unequivocally assigned from all combinations of genotypes except for those individuals heterozygous at both IVS5 and EX8. For these individuals the haplotypes are ambiguous and for this reason, the critical haplotype associated with the lowest level of HDL-C and highest TG could not be precisely defined.

The combined IVS5 and EX8 genotypes are an excellent marker of an underlying genetic variant(s) in CD36L1 leading to an elevated TG:HDL-C ratio. The six SNPs evaluated included one common (EX1) and one rare (EX3) missense variant, neither of which could account for the observed association. Further investigation of candidate variants in intronic and regulatory regions, and the construction of haplotypes across the gene may reveal the causative variant(s).

CD36L1 variants associated with serum lipids appear to act in a sex-dependent manner. Significant gene by sex interaction was found for both combinations of genotypes, revealing an underlying association in women, but not in men. It is well known that HDL-C levels are affected by sex hormone status. Furthermore, the expression of CD36L1 is known to be regulated by estrogen. Estrogen treatment of rats has been shown to down regulate CD36L1 in the liver (Flutier, et al. (1998) J Biol Chem 273:8434-8; Landschulz, et al. (1996) J Clin Invest 98(4):984-95). Moreover, overexpression of CD36L1 in the liver has been demonstrated to result in a pronounced fall in plasma HDL-C (Kozarsky, et al. (1997) Nature 387-414-7). Regulation of CD36L1 by estrogen may be influenced by genetic variants in CD36L1, which may have implications for the treatment of postmenopausal women with hormone replacement therapy (HRT). CD36L1 variants may modulate the effect of HRT on HDL-C levels in women.

The interaction between the CD36L1 variants and hormonal status has implications for the treatment of females with hormone replacement therapy (HRT). Females who take HRT have an increased risk for vascular disease, e.g., CAD. In females, the identification of CD34L1 variants associated with abnormal lipid levels, e.g., high TG levels or high TG:HDL-C ratios may be used to predict the effect HRT would have on lipid levels (e.g., increasing TG level and/or lowering HDL level). Accordingly, the CD36L1 genotype may also be used as a pharmacogenomic marker of response to HRT.

None of the individual variants in CD36L1 alone revealed an association with serum lipids. An association was only apparent when combinations of SNPs were examined, taking into account an interaction with sex. Once the interactions were taken into account, the association was strong and highly significant. This underscores the complex nature of dyslipidemias and illustrates the challenges encountered in uncovering and reproducing genetic associations.

The polymorphisms described herein are single nucleotide polymorphisms (SNPs) at a specific nucleotide residues within the CD36L1 gene. The CD36L1 gene has at least two alleles, referred to herein as the reference allele and the variant allele. The reference allele (i.e., the consensus sequence, or wild type allele) has been designated based on it's frequency in a general U.S. Caucasian population sample. The reference allele is the more common of the two alleles; the variant is the more rare of the two alleles.

It is understood that the invention is not limited by this exemplified reference sequence, as variants of this sequence which differ at locations other than the SNP site identified herein can also be utilized. The skilled artisan can readily determine the SNP sites in these other reference sequences which correspond to the SNP site identified herein by aligning the sequence of interest with the reference sequences specifically disclosed herein, and programs for performing such alignments are commercially available. For example, the ALIGN program in the GCG software package can be used, utilizing a PAM120 weight residue table, a gap length penalty of 12 and a gap penalty of 4, for example.

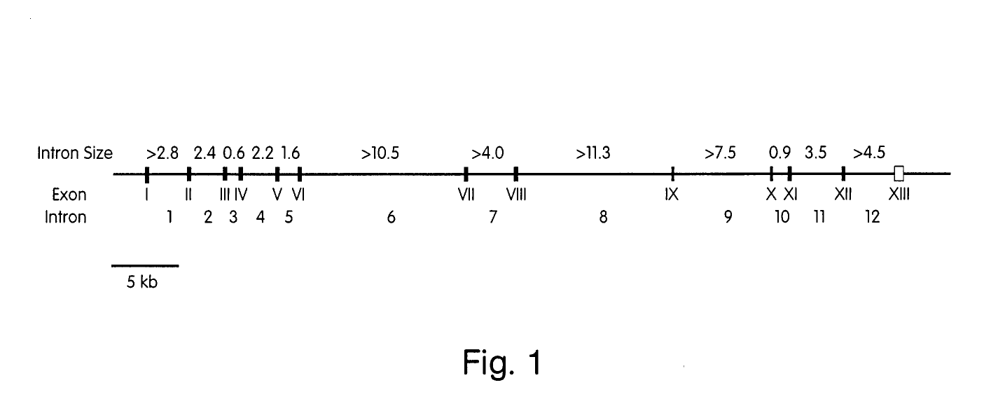

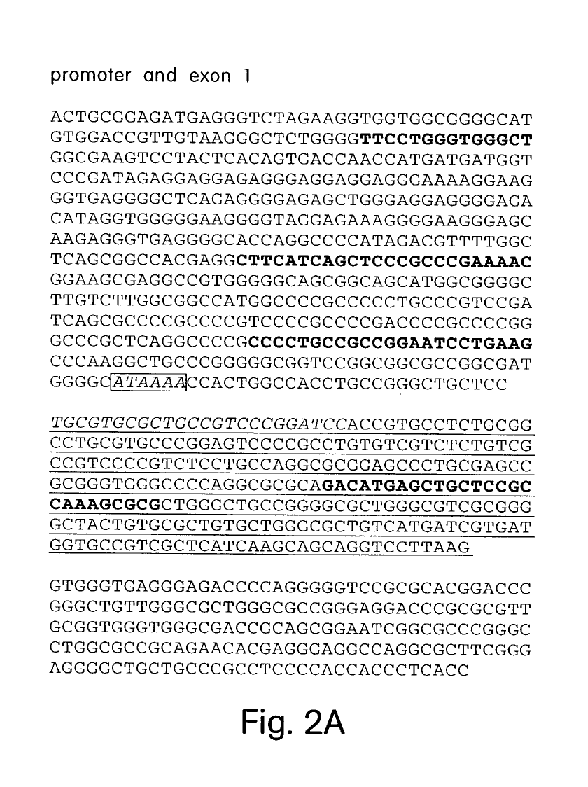

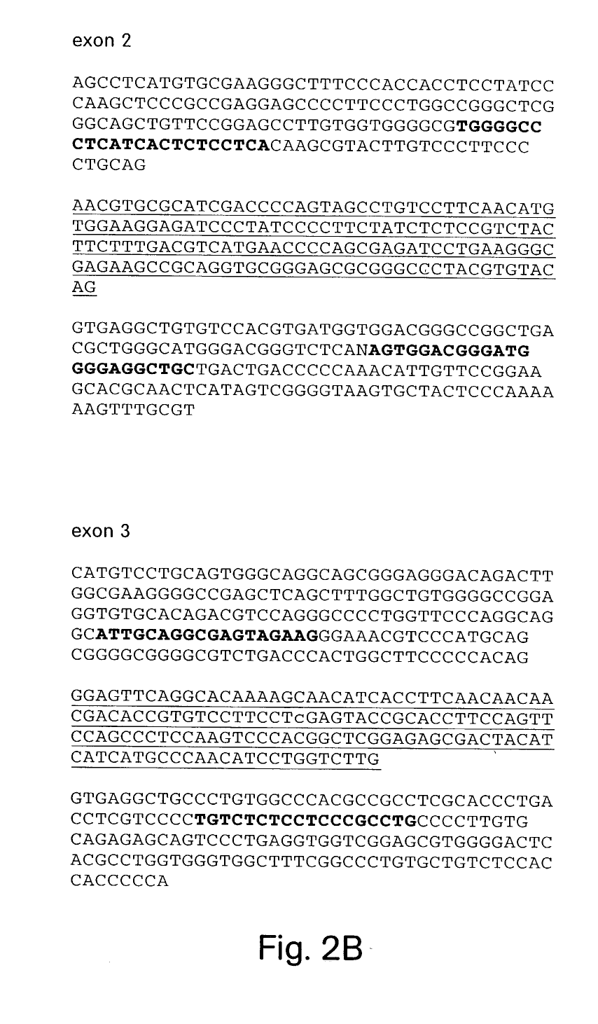

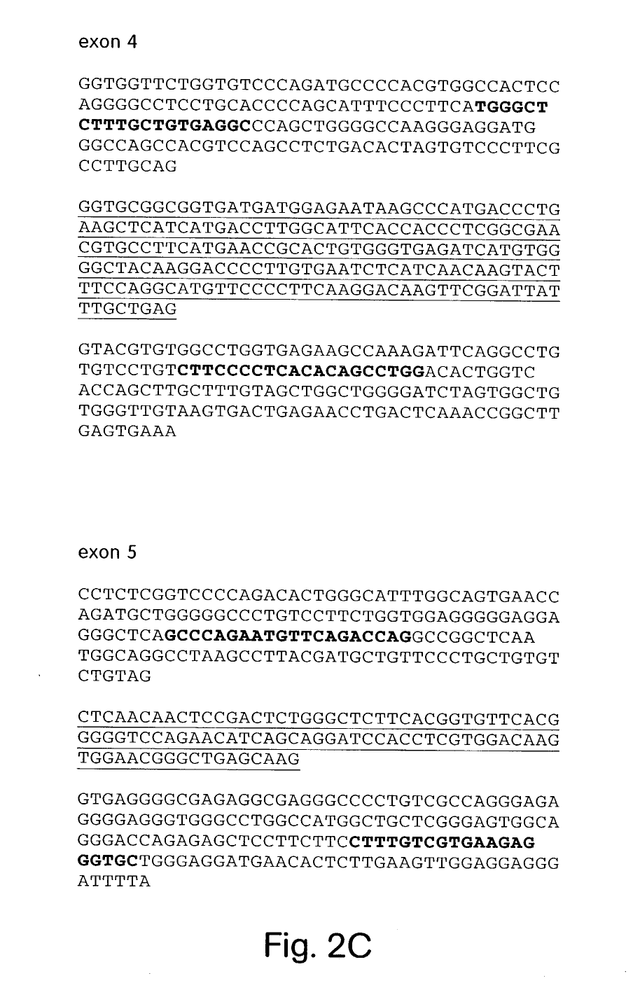

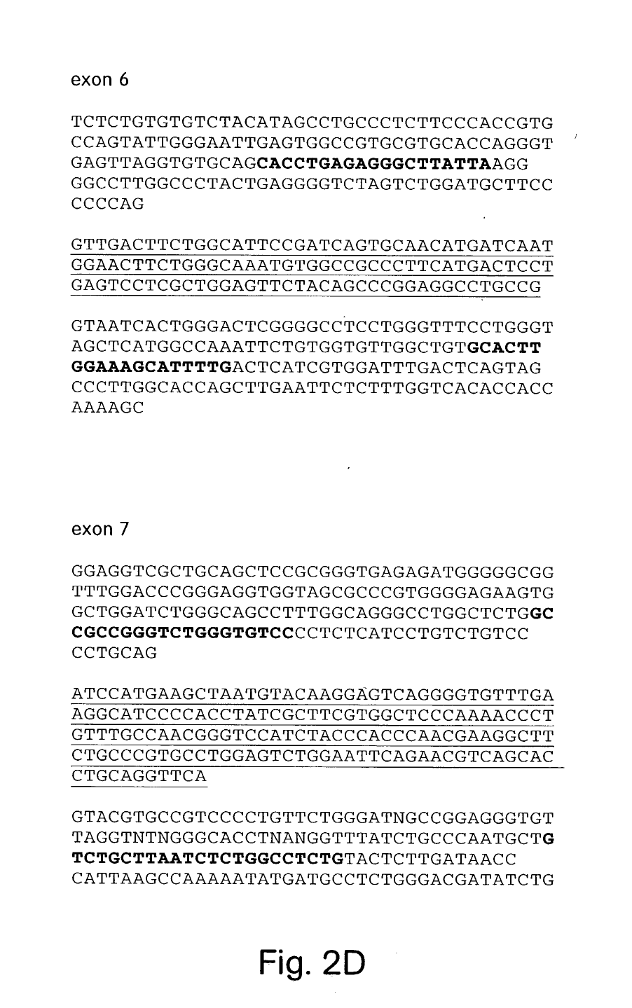

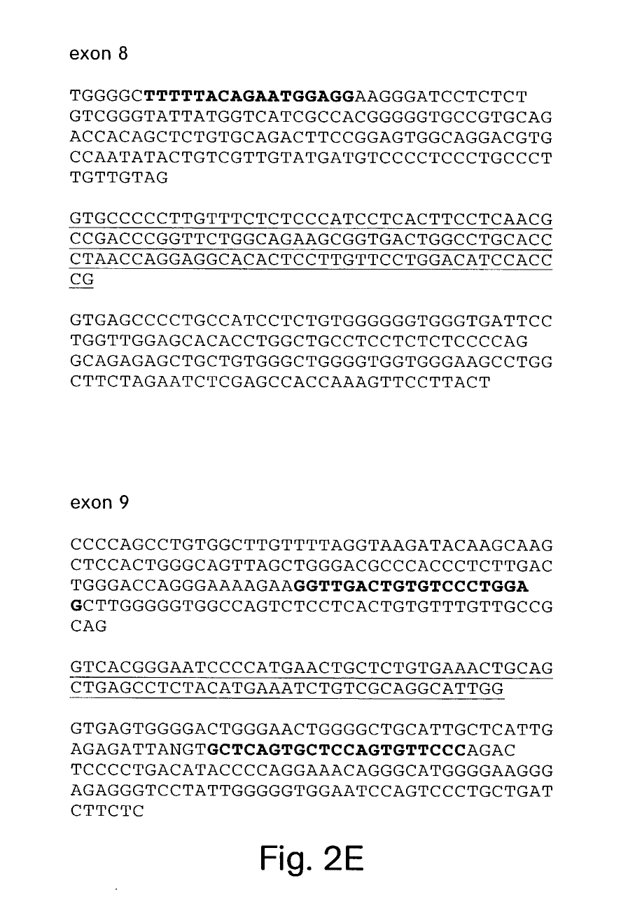

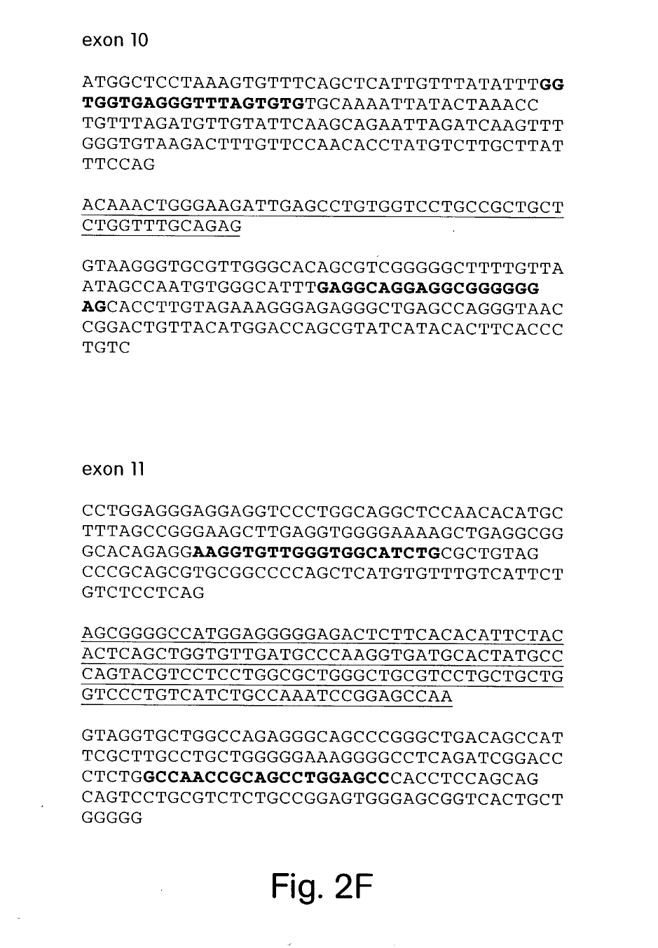

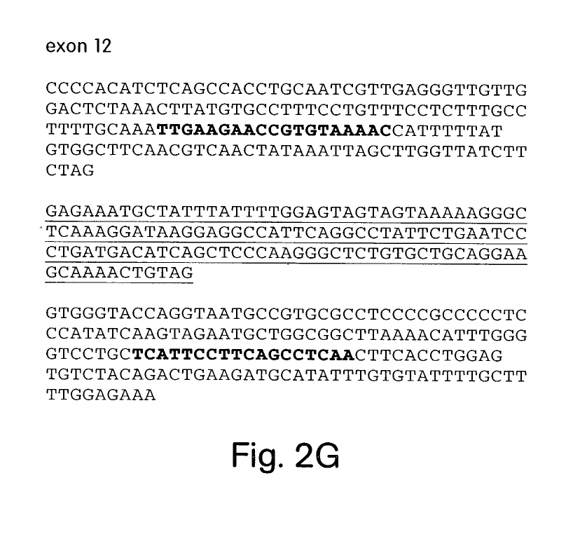

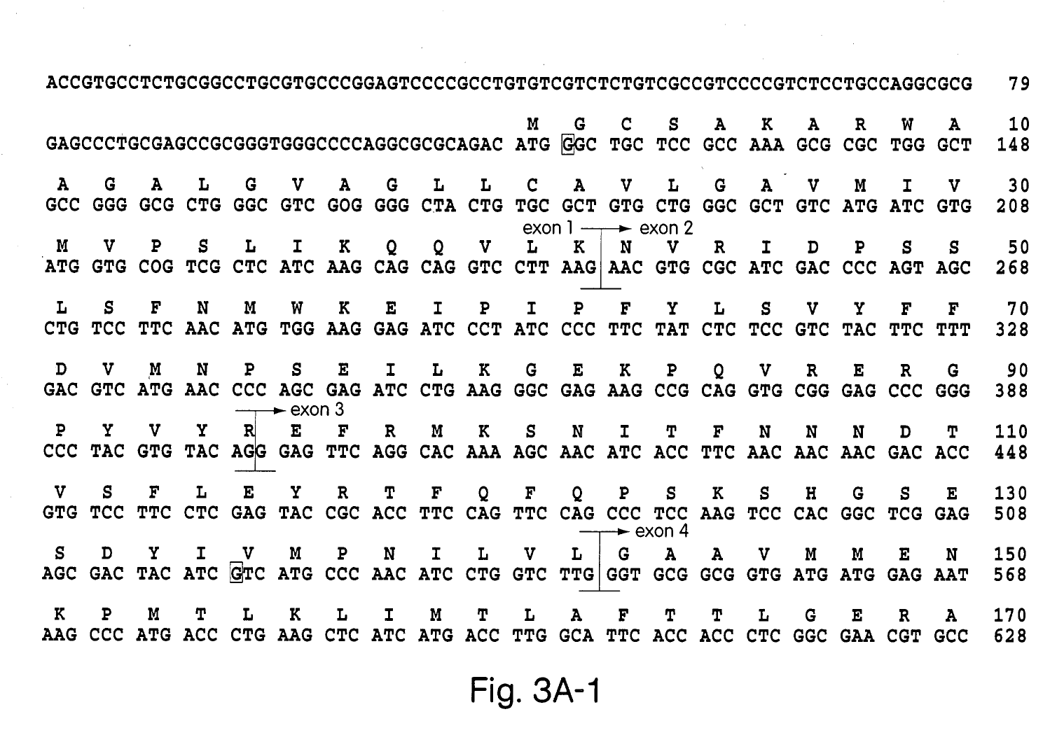

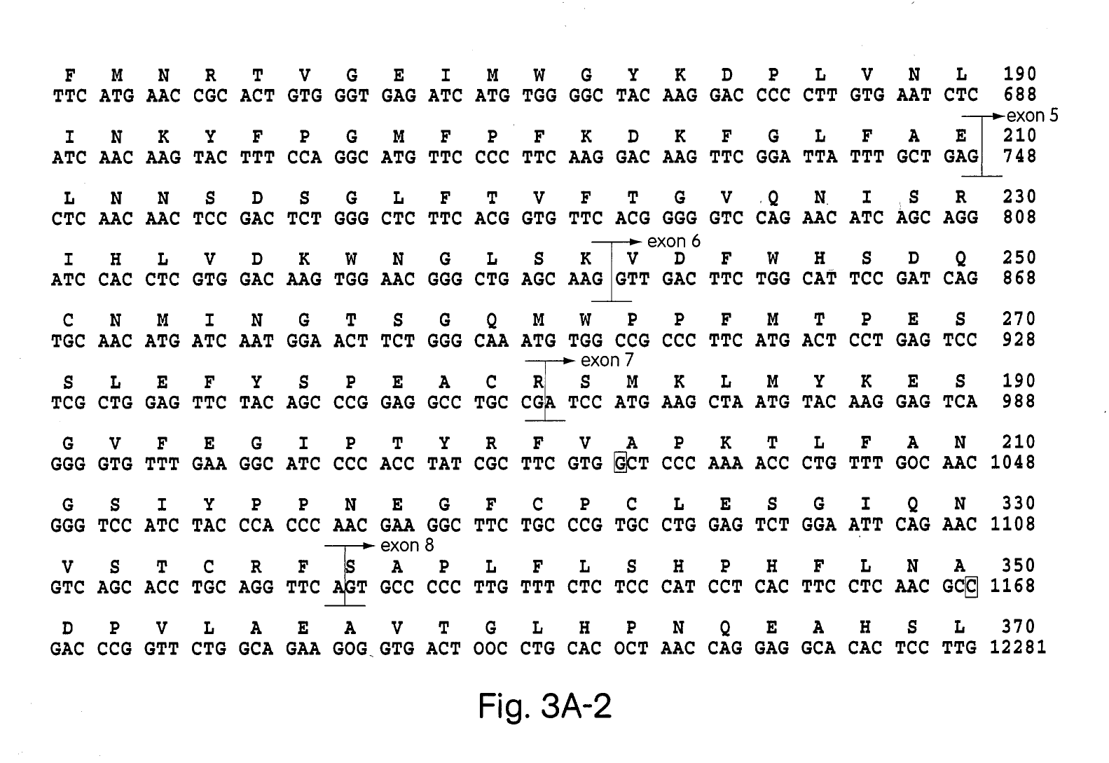

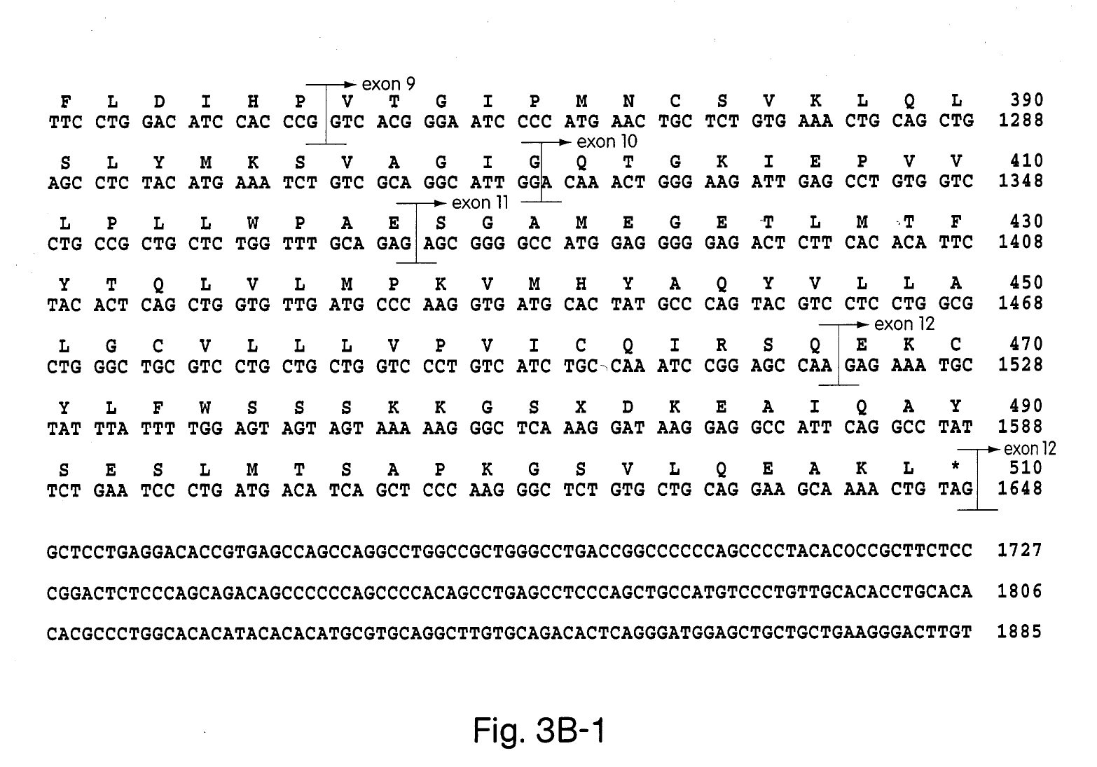

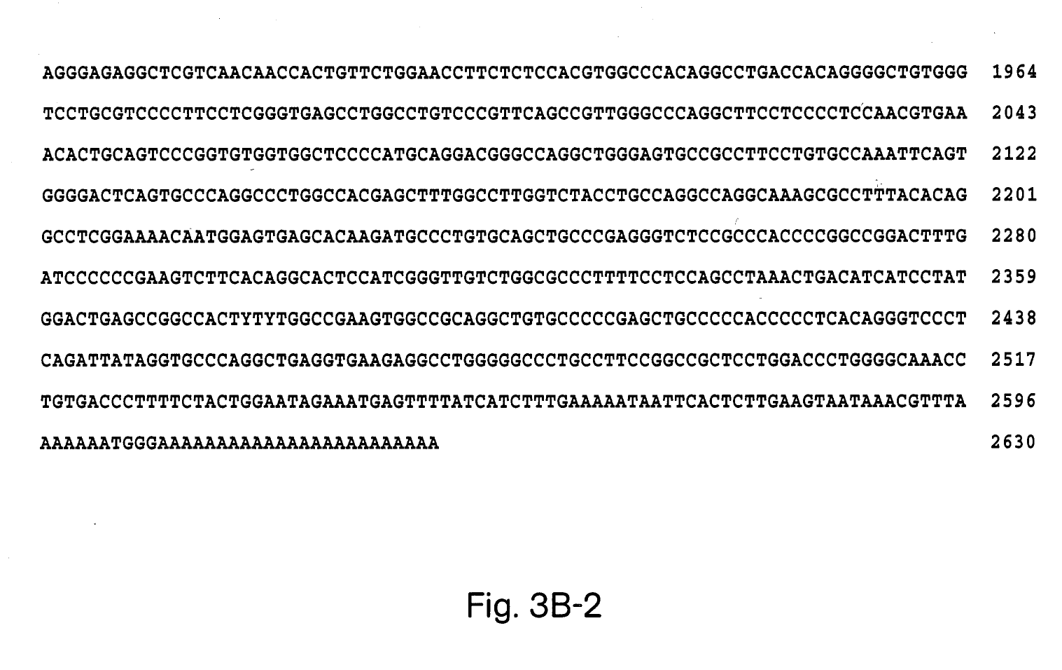

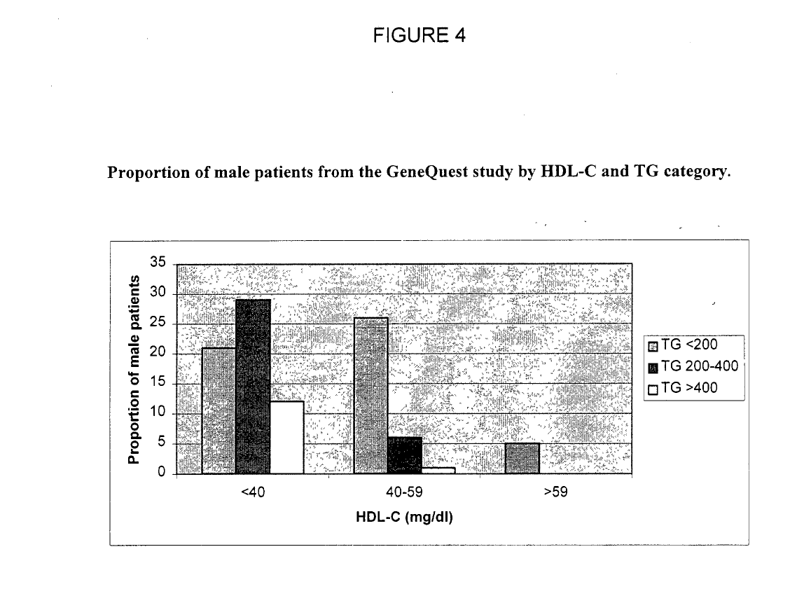

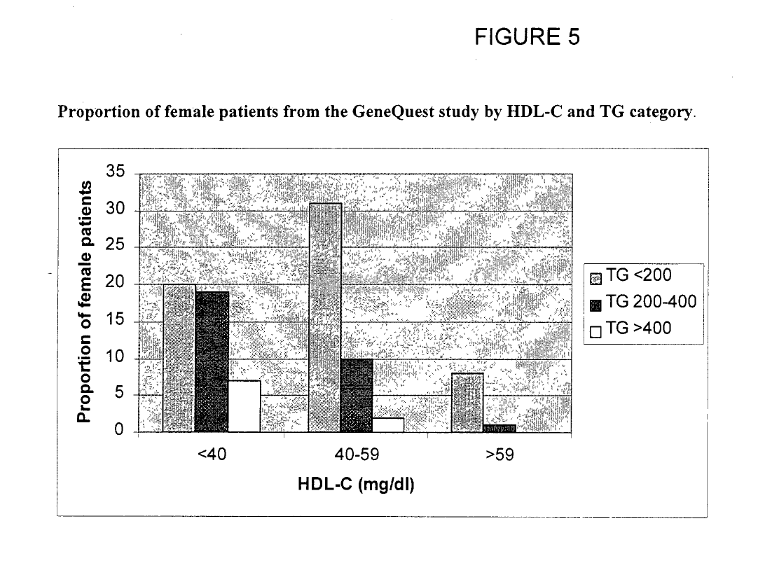

As shown in FIG. 1, the human CD36L1 gene is at least 50 kilobase pairs long and has 12 coding exons, one non-coding exon (exon 13), and 12 introns. The exons are numbered 1 to 13 from 5′ to 3′ and the introns are labeled 1 through 12 from 5′ to 3′. Exon 1 corresponds to the first exon located downstream of the promoter and contains the initiation codon. Intron 1 is located immediately downstream of exon 1 (see FIG. 1). The position of the introns relative to the nucleotide sequence of the full length cDNA encoding CD36L1 is shown in FIG. 2A-G. The nucleotide sequence of the human CD36L1 cDNA, shown in FIG. 3 and in SEQ ID NO: 1 encodes a protein of 509 amino acids. SEQ ID NO:1 contains the nucleotide sequence of the cDNA disclosed in Calvo and Vega (1993) J. Biol. Chem. 268:18929, and contains in addition a complete 5′ end. The amino acid sequence of the protein set forth in SEQ ID NO: 2 is identical to the Cla-I protein disclosed in Calvo and Vega (1993) J. Biol. Chem. 268:18929. As set forth in Calvo and Vega, supra, differential splicing of the human CD36L1 gene also results in a short mRNA lacking 300 nucleotides located 126 nucleotides downstream of the initiation codon, i.e., lacking exons 2 and 3 set forth in FIG. 3, which encodes a protein of 409 amino acids. The shorter protein is referred to herein as “splice variant”. The nucleotide sequence of a full length cDNA encoding the splice variant is set forth in SEQ ID NO: 3 and the amino acid sequence of the CD36L1 splice variant protein encoded by this nucleotide sequence is set forth in SEQ ID NO: 4. The splice variant is rare relative to the 509 amino acid CD36L1 protein.

FIG. 2A-G shows the nucleotide sequence of the 3′ end of the CD36L1 promoter. Additional promoter sequence is disclosed in U.S. Pat. No. 5,965,790 by Acton, incorporated herein by reference.

Set forth below in Table II are the locations and sizes of the exons in the human CD36L1 gene relative to the nucleotide sequence of a full length cDNA encoding human CD36L1 protein (SEQ ID NO: 1), in which nucleotide 1 corresponds to the first nucleotide in the isolated transcript. Table II also indicates the portions of the human CD36L1 protein encoded by each of these exons. Amino acid 1 is the initiating methionine. Also indicated is the length of the intron located downstream of each of the exons.

TABLE II

| | cDNA | | | |
|---|---|---|---|---|
| | Nucleotide position | Amino acid position | Size of intron | |
| Exon 1 | 1-244 | 1-42 | intron 1: | >2827 |
| Exon 2 | 245-402 | 43-95 | intron 2: | 2429 |
| Exon 3 | 403-544 | 95-142 | intron 3: | 567 |
| Exon 4 | 545-748 | 143-210 | intron 4: | 2229 |
| Exon 5 | 749-844 | 211-242 | intron 5: | 1580 |
| Exon 6 | 845-960 | 243-281 | intron 6: | >10532 |
| Exon 7 | 961-1127 | 281-337 | intron 7: | >3985 |
| Exon 8 | 1228-1246 | 337-376 | intron 8: | >11321 |
| Exon 9 | 1247-1320 | 377-401 | intron 9: | 7562 |
| Exon 10 | 1321-1372 | 401-418 | intron 10: | 902 |
| Exon 11 | 1373-1519 | 419-467 | intron 11: | 3547 |
| Exon 12 | 1520-1648 | 468-509 | intron 12: | >4578 |
| Exon 13 | 1649-2630 | | | |

FIG. 2A-G shows the nucleotide sequence of portions of the introns which are adjacent to the exons. The nucleotide sequence of each of the exons and adjacent portions of introns shown in FIG. 2A-G are set forth in SEQ ID Nos. 5 to 16. The portions of each of the introns shown in FIG. 2A-G are set forth in SEQ ID Nos. 18 to 40. For convenience, the identity of the sequences referred to as SEQ ID Nos. 1 to 40 are set forth below in Table III:

TABLE III

| | |
|---|---|
| SEQ ID NO: 1 | full length cDNA encoding human CD36L1; |
| SEQ ID NO: 2 | full length amino acid sequence of human CD36L1 protein; |
| SEQ ID NO: 3 | full length cDNA encoding splice variant of human CD36L1 (Calvo |
| and | Vega, supra); |
| SEQ ID NO: 4 | full length amino acid sequence of splice variant of human CD36L1 |
| | protein (Calvo and Vega, supra); |
| SEQ ID NO: 5 | 3′ end of promoter, exon 1, and 5′ end of intron 1; |
| SEQ ID NO: 6 | 3′ end of intron 1, exon 2, and 5′ end of intron 2; |
| SEQ ID NO: 7 | 3′ end of intron 2, exon 3, and 5′ end of intron 3; |
| SEQ ID NO: 8 | 3′ end of intron 3, exon 4, and 5′ end of intron 4; |
| SEQ ID NO: 9 | 3′ end of intron 4, exon 5, and 5′ end of intron 5; |
| SEQ ID NO: 10 | 3′ end of intron 5, exon 6, and 5′ end of intron 6; |
| SEQ ID NO: 11 | 3′ end of intron 6, exon 7, and 5′ end of intron 7; |
| SEQ ID NO: 12 | 3′ end of intron 7, exon 8, and 5′ end of intron 8; |
| SEQ ID NO: 13 | 3′ end of intron 8, exon 9, and 5′ end of intron 9; |
| SEQ ID NO: 14 | 3′ end of intron 9, exon 10, and 5′ end of intron 10; |
| SEQ ID NO: 15 | 3′ end of intron 10 ,exon 11, and 5′ end of intron 11; |
| SEQ ID NO: 16 | 3′ end of intron 11, exon 12, and 5′ end of intron 12; |
| SEQ ID NO: 17 | 3′ end of promoter; |
| SEQ ID NO: 18 | 5′ end of intron 1; |
| SEQ ID NO: 19 | 3′ end of intron 1; |
| SEQ ID NO: 20 | 5′ end of intron 2; |
| SEQ ID NO: 21 | 3′ end of intron 2; |
| SEQ ID NO: 22 | 5′ end of intron 3; |
| SEQ ID NO: 23 | 3′ end of intron 3; |
| SEQ ID NO: 24 | 5′ end of intron 4; |
| SEQ ID NO: 25 | 3′ end of intron 4; |
| SEQ ID NO: 26 | 5′ end of intron 5; |
| SEQ ID NO: 27 | 3′ end of intron 5; |
| SEQ ID NO: 28 | 5′ end of intron 6; |
| SEQ ID NO: 29 | 3′ end of intron 6; |
| SEQ ID NO: 30 | 5′ end of intron 7; |
| SEQ ID NO: 31 | 3′ end of intron 7; |
| SEQ ID NO: 32 | 5′ end of intron 8; |
| SEQ ID NO: 33 | 3′ end of intron 8; |
| SEQ ID NO: 34 | 5′ end of intron 9; |
| SEQ ID NO: 35 | 3′ end of intron 9; |
| SEQ ID NO: 36 | 5′ end of intron 10; |
| SEQ ID NO: 37 | 3′ end of intron 10; |
| SEQ ID NO: 38 | 5′ end of intron 11; |
| SEQ ID NO: 39 | 3′ end of intron 11; and |
| SEQ ID NO: 40 | 5′ end of intron 12. |

The nucleic acid molecules of the invention can be double- or single-stranded. Accordingly, the invention further provides for the complementary nucleic acid strands comprising the polymorphisms described herein.

Other aspects of the invention are described below or will be apparent to one of skill in the art in light of the present disclosure.

Definitions

For convenience, the meaning of certain terms and phrases employed in the specification, examples, and appended claims are provided below.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene, including the CD36L1 gene, can differ from each other in a single nucleotide, or several nucleotides, and can include substitutions, deletions, and insertions of nucleotides. An allele of a gene can also be a form of a gene containing one or more mutations.

The term “allelic variant of a polymorphic region of a CD36L1 gene” refers to an alternative form of the CD36L1 gene having one of several possible nucleotide sequences found in that region of the gene in the population.

“Biological activity” or “bioactivity” or “activity” or “biological function”, which are used interchangeably, for the purposes herein when applied to CD36L1 means an effector or antigenic function that is directly or indirectly performed by a CD36L1 polypeptide (whether in its native or denatured conformation), or by any subsequence (fragment) thereof. Biological activities include binding to a ligand, e.g., a lipid or lipoprotein, such as LDL or modified forms thereof, or HDL or modified forms thereof. Other molecules which can bind a CD36L1 receptor include anionic molecules, such as anionic phospholipids, negatively charged liposomes, and apoptotic cells. Another biological activity of a CD36L1 protein includes endocytosis of a ligand interacting with the receptor. A biological activity is also intended to include binding to a protein, such as binding to the cytoplasmic domain of CD36L1. Yet other biological activities include signal transduction from the receptor, modulation of expression of genes responsive to binding of a ligand to a CD36L1 receptor, and other biological activities, whether presently known or inherent. A CD36L1 bioactivity can be modulated by directly affecting a CD36L1 protein. Alternatively, a CD36L1 bioactivity can be modulated by modulating the level of a CD36L1 protein, such as by modulating expression of a CD36L1 gene. Antigenic functions include possession of an epitope or antigenic site that is capable of cross-reacting with antibodies raised against a naturally occurring or denatured CD36L1 polypeptide or fragment thereof.

Biologically active CD36L1 polypeptides include polypeptides having both an effector and antigenic function, or only one of such functions. CD36L1 polypeptides include antagonist polypeptides and native CD36L1 polypeptides, provided that such antagonists include an epitope of a native CD36L1 polypeptide. An effector function of CD36L1 polypeptide can be the ability to bind to a ligand, e.g., a lipid or modified form thereof.

As used herein the term “bioactive fragment of a CD36L1 protein” refers to a fragment of a full-length CD36L1 protein, wherein the fragment specifically mimics or antagonizes the activity of a wild-type CD36L1 protein. The bioactive fragment preferably is a fragment capable of binding to a second molecule, such as a ligand.

The term “an aberrant activity” or “abnormal activity”, as applied to an activity of a protein such as CD36L1, refers to an activity which differs from the activity of the normal or reference protein or which differs from the activity of the protein in a healthy subject, e.g., a subject not afflicted with a disease associated with a CD36L1 allelic variant. An activity of a protein can be aberrant because it is stronger than the activity of its wild-type counterpart. Alternatively, an activity of a protein can be aberrant because it is weaker or absent relative to the activity of its normal or reference counterpart. An aberrant activity can also be a change in reactivity. For example an aberrant protein can interact with a different protein or ligand relative to its normal or reference counterpart. A cell can also have aberrant CD36L1 activity due to overexpression or underexpression of the CD36L1 gene. Aberrant CD36L1 activity can result from a mutation in the gene, which results, e.g., in lower or higher binding affinity of a ligand to the CD36L1 protein encoded by the mutated gene. Aberrant CD36L1 activity can also result from an abnormal CD36L1 5′ upstream regulatory element activity.

“Cells,” “host cells” or “recombinant host cells” are terms used interchangeably herein. It is understood that such terms refer not only to the particular cell but to the progeny or derivatives of such a cell. Because certain modifications may occur in succeeding generations due to either mutation or environmental influences, such progeny may not, in fact, be identical to the parent cell, but are still included within the scope of the term as used herein.

As used herein, the term “course of clinical therapy” refers to any chosen method to treat, prevent, or ameliorate abnormal lipid levels, e.g., abnormally high TG level or an abnormally high TG:HDL-C ratio, or a disease or disorder associated with abnormal lipid levels, e.g., a vascular or metabolic disease or disorder, symptoms thereof, or related diseases or disorders. Courses of clinical therapy include, but are not limited to, lifestyle changes (e.g., changes in diet, exercise, or environment), administration of medication, e.g., lipid modulating medication. Clinical course of therapy for treatment or prevention or amelioration of vascular disease in particular includes, for example, use of medical devices, such as, but not limited to, a defibrillator, a stent, a device used in coronary revascularization, a pacemaker, or any combination thereof, and surgical procedures such as percutaneous transluminal coronary balloon angioplasty (PTCA) or laser angioplasty, or other surgical intervention, such as, for example, coronary bypass grafting (CABG), or any combination thereof.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

As used herein, the term “genetic profile” refers to the information obtained from identification of the specific allelic variants of a subject. For example, a CD36L1 genetic profile refers to the specific allelic variants of a subject within the CD36L1 gene. For example, one can determine a subject's CD36L1 genetic profile by determining the identity of one or more of the nucleotides present at nucleotide residue 41 of exon 8 (the EX8 polymorphism) and nucleotide residue 54 of intron 5 (the IVS5 polymorphism) of the CD36L1 gene, or the complements thereof. The genetic profile of a particular disease can be ascertained through identification of the identity of allelic variants in one or more genes which are associated with the particular disease.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, (1988) CABIOS4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The term “a homolog of a nucleic acid” refers to a nucleic acid having a nucleotide sequence having a certain degree of homology with the nucleotide sequence of the nucleic acid or complement thereof. For example, a homolog of a double stranded nucleic acid having SEQ ID NO:N is intended to include nucleic acids having a nucleotide sequence which has a certain degree of homology with SEQ ID NO:N or with the complement thereof. Preferred homologs of nucleic acids are capable of hybridizing to the nucleic acid or complement thereof.

The term “hybridization probe” or “primer” as used herein is intended to include oligonucleotides which hybridize bind in a base-specific manner to a complementary strand of a target nucleic acid. Such probes include peptide nucleic acids, and described in Nielsen et al., (1991) Science 254:1497-1500. Probes and primers can be any length suitable for specific hybridization to the target nucleic acid sequence. The most appropriate length of the probe and primer may vary depending on the hybridization method in which it is being used; for example, particular lengths may be more appropriate for use in microfabricated arrays, while other lengths may be more suitable for use in classical hybridization methods. Such optimizations are known to the skilled artisan. Suitable probes and primers can range form about 5 nucleotides to about 30 nucleotides in length. For example, probes and primers can be 5, 6, 8, 10, 12, 14, 16, 18, 20, 22, 24, 25, 26, 28 or 30 nucleotides in length. The probe or primer of the invention comprises a sequence that flanks and/or preferably overlaps, at least one polymorphic site occupied by any of the possible variant nucleotides. The nucleotide sequence of an overlapping probe or primer can correspond to the coding sequence of the allele or to the complement of the coding sequence of the allele.

A “disease or disorder associated with abnormal TG levels and/or TG:HDL-C ratio” or a “disease or disorder associated with abnormal lipid levels,” as used herein, includes any disease or disorder for which abnormal lipid levels, e.g, abnormal TG levels and/or abnormal TG:HDL-C ratio, is a risk factor, e.g., a vascular or metabolic disease or disorder. The term “vascular disease or disorder” as used herein refers to any disease or disorder effecting the vascular system, including the heart and blood vessels. A vascular disease or disorder includes any disease or disorder characterized by vascular dysfunction, including, for example, intravascular stenosis (narrowing) or occlusion (blockage), due to the development of atherosclerotic plaque and diseases and disorders resulting therefrom. Examples of vascular diseases and disorders include, without limitation, abnormal lipid metabolism, abnormal lipid level, abnormal TG level, abnormal TG:HDL-C ratio, atherosclerosis, CAD, MI, ischemia, stroke, peripheral vascular diseases, venous thromboembolism and pulmonary embolism.

As used herein, the term “metabolic disease or disorder” includes a disorder, disease or condition which is caused or characterized by an abnormal metabolism (i.e., the chemical changes in living cells by which energy is provided for vital processes and activities) in a subject. Metabolic diseases and disorders include diseases, disorders, or conditions associated with abnormal lipid levels, e.g., abnormal TG or HDL-C levels. Examples of metabolic diseases and disorders include obesity, diabetes, hyperphagia, endocrine abnormalities, triglyceride storage disease, Bardet-Biedl syndrome, Lawrence-Moon syndrome, Prader-Labhart-Willi syndrome, anorexia, and cachexia. Obesity is defined as a body mass index (BMI) of 30 kg/²m or more (National Institute of Health, Clinical Guidelines on the Identification, Evaluation, and Treatment of Overweight and Obesity in Adults (1998)). However, the present invention is also intended to include a disease, disorder, or condition that is characterized by a body mass index (BMI) of 25 kg/²m or more, 26 kg/²m or more, 27 kg/²m or more, 28 kg/²m or more, 29 kg/²m or more, 29.5 kg/²m or more, or 29.9 kg/²m or more, all of which are typically referred to as overweight (National Institute of Health, Clinical Guidelines on the Identification, Evaluation, and Treatment of Overweight and Obesity in Adults (1998)).

As used herein, the term “abnormally high TG level” refers to a TG level which is higher than the level generally accepted by one of skill in the art as being normal, e.g., higher than 200 mg/dl. An exceptionally high TG level is a TG level which is higher than 400 mg/dl.

As used herein, the term “abnormally high TG:HDL-C ratio” refers to a TG:HDL-C ratio which is higher than the level generally accepted by one of skill in the art as being normal, e.g., TG:HDL-C>1 (logTG:HDL-C>0). Values above 1.0 also correlate with the atherogenic lipoprotein particle type B. TG:HDL-C ratios below 1.0 correspond to a low risk of CAD (see Dobiasova, et al. (2001) Clin Biochem 34(7):583-8). An exceptionally high TG:HDL-C ratio is a ratio which is higher than 7.0.

The term “interact” as used herein with respect to interaction between molecules, is meant to include detectable interactions between molecules, such as can be detected using, for example, a binding or hybridization assay. The term interact is also meant to include “binding” interactions between molecules. Interactions may be, for example, protein-protein, protein-nucleic acid, protein-small molecule or small molecule-nucleic acid in nature. The term “interaction” when used in the context of a statistical relationship or analysis, refers to a means for demonstrating the underlying effect of haplotypes, e.g., the combined effect of SNPs at different loci that are in LD.

The term “intronic sequence” or “intronic nucleotide sequence” refers to the nucleotide sequence of an intron or portion thereof.

The term “isolated” as used herein with respect to nucleic acids, such as DNA or RNA, refers to molecules separated from other DNAs or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “lipid” refers to a fat or fat-like substance that is insoluble in polar solvents such as water. The term “lipid” is intended to include true fats (e.g esters of fatty acids and glycerol); lipids (phospholipids, cerebrosides, waxes); sterols (cholesterol, ergosterol) and lipoproteins (e.g. triglyceride, HDL, LDL and VLDL).

The term “linkage” describes the tendency of genes, alleles, loci or genetic markers to be inherited together as a result of their location on the same chromosome. It can be measured by percent recombination between the two genes, alleles, loci, or genetic markers. The term “linkage disequilibrium,” also referred to herein as “LD,” refers to a greater than random association between specific alleles at two marker loci within a particular population. In general, linkage disequilibrium decreases with an increase in physical distance. If linkage disequilibrium exists between two markers, then the genotypic information at one marker can be used to make probabilistic predictions about the genotype of the second marker.

The term “locus” refers to a specific position in a chromosome. For example, a locus of a CD36L1 gene refers to the chromosomal position of the CD36L1 gene.

The term “modulation” as used herein refers to both upregulation, (i.e., activation or stimulation), for example by agonizing; and downregulation (i.e., inhibition or suppression), for example by antagonizing of a bioactivity (e.g. expression of a gene).

The term “molecular structure” of a gene or a portion thereof refers to the structure as defined by the nucleotide content (including deletions, substitutions, additions of one or more nucleotides), the nucleotide sequence, the state of methylation, and/or any other modification of the gene or portion thereof.

The term “mutated gene” refers to an allelic form of a gene that differs from the predominant form in a population. A mutated gene is capable of altering the phenotype of a subject having the mutated gene relative to a subject having the predominant form of the gene. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenine”, “cytidine”, “guanine”, and thymidine” and/or “A”, “C”, “G”, and “T”, respectively, are used. It is understood that if the nucleic acid is RNA, a nucleotide having a uracil base is uridine.

The term “nucleotide sequence complementary to the nucleotide sequence set forth in SEQ ID NO:N” refers to the nucleotide sequence of the complementary strand of a nucleic acid strand having SEQ ID NO:N. The term “complementary strand” is used herein interchangeably with the term “complement.” The complement of a nucleic acid strand can be the complement of a coding strand or the complement of a non-coding strand. When referring to double stranded nucleic acids, the complement of a nucleic acid having SEQ ID NO:N refers to the complementary strand of the strand having SEQ ID NO:N or to any nucleic acid having the nucleotide sequence of the complementary strand of SEQ ID NO:N. When referring to a single stranded nucleic acid having the nucleotide sequence SEQ ID NO:N, the complement of this nucleic acid is a nucleic acid having a nucleotide sequence which is complementary to that of SEQ ID NO:N. The nucleotide sequences and complementary sequences thereof are always given in the 5′ to 3′ direction. The term “complement” and “reverse complement” are used interchangeably herein.

A “non-human animal” of the invention can include mammals such as rodents, non-human primates, sheep, goats, horses, dogs, cows, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which an exogenous sequence is found, or in which an exogenous sequence is expressed in some but not all cells of the animal. The term “tissue-specific chimeric animal” indicates that an exogenous sequence is present and/or expressed or disrupted in some tissues, but not others.

The term “oligonucleotide” is intended to include and single- or double stranded DNA or RNA. Oligonucleotides can be naturally occurring or synthetic, but are typically prepared by synthetic means. Preferred oligonucleotides of the invention include segments of CD36L1 gene sequence or their complements, which include and/or flank any one of the polymorphic sites described herein. The segments can be between 5 and 250 bases, and, in specific embodiments, are between 5-10, 5-20, 10-20, 10-50, 20-50 or 10-100 bases. For example, the segments can be 21 bases. The polymorphic site can occur within any position of the segment or a region next to the segment. The segments can be from any of the allelic forms of the CD36L1 gene sequences described herein.

The term “operably-linked” is intended to mean that the 5′ upstream regulatory element is associated with a nucleic acid in such a manner as to facilitate transcription of the nucleic acid from the 5′ upstream regulatory element.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic locus can be a single nucleotide, the identity of which differs in the other alleles. A polymorphic locus can also be more than one nucleotide long. The allelic form occurring most frequently in a selected population is often referred to as the reference and/or wildtype form. Other allelic forms are typically designated or alternative or variant alleles. Diploid organisms may be homozygous or heterozygous for allelic forms. A diallelic or biallelic polymorphism has two forms. A trialleleic polymorphism has three forms.

A “polymorphic gene” refers to a gene having at least one polymorphic region.

The term “primer” as used herein, refers to a single-stranded oligonucleotide which acts as a point of initiation of template-directed DNA synthesis under appropriate conditions (e.g., in the presence of four different nucleoside triphosphates and as agent for polymerization, such as DNA or RNA polymerase or reverse transcriptase) in an appropriate buffer and at a suitable temperature. The length of a primer may vary but typically ranges from 15 to 30 nucleotides. A primer need not match the exact sequence of a template, but must be sufficiently complementary to hybridize with the template.

The term “primer pair” refers to a set of primers including an upstream primer that hybridizes with the 3′ end of the complement of the DNA sequence to be amplified and a downstream primer that hybridizes with the 3′ end of the sequence to be amplified. The terms “protein”, “polypeptide” and “peptide” are used interchangeably herein when referring to a gene product.

The term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

A “regulatory element”, also termed herein “regulatory sequence” is intended to include elements which are capable of modulating transcription from a 5′ upstream regulatory sequence, including, but not limited to a basic promoter, and include elements such as enhancers and silencers. The term “enhancer”, also referred to herein as “enhancer element”, is intended to include regulatory elements capable of increasing, stimulating, or enhancing transcription from a 5′ upstream regulatory element, including a basic promoter. The term “silencer”, also referred to herein as “silencer element” is intended to include regulatory elements capable of decreasing, inhibiting, or repressing transcription from a 5′ upstream regulatory element, including a basic promoter. Regulatory elements are typically present in 5′ flanking regions of genes. Regulatory elements also may be present in other regions of a gene, such as introns. Thus, it is possible that a CD36L1 gene has regulatory elements located in introns, exons, coding regions, and 3′ flanking sequences. Such regulatory elements are also intended to be encompassed by the present invention and can be identified by any of the assays that can be used to identify regulatory elements in 5′ flanking regions of genes.

The term “regulatory element” further encompasses “tissue specific” regulatory elements, i.e., regulatory elements which effect expression of an operably linked DNA sequence preferentially in specific cells (e.g., cells of a specific tissue). Gene expression occurs preferentially in a specific cell if expression in this cell type is significantly higher than expression in other cell types. The term “regulatory element” also encompasses non-tissue specific regulatory elements, i.e., regulatory elements which are active in most cell types. Furthermore, a regulatory element can be a constitutive regulatory element, i.e., a regulatory element which constitutively regulates transcription, as opposed to a regulatory element which is inducible, i.e., a regulatory element which is active primarily in response to a stimulus. A stimulus can be, e.g., a molecule, such as a protein, hormone, cytokine, heavy metal, phorbol ester, cyclic AMP (cAMP), or retinoic acid.

Regulatory elements are typically bound by proteins, e.g., transcription factors. The term “transcription factor” is intended to include proteins or modified forms thereof, which interact preferentially with specific nucleic acid sequences, i.e., regulatory elements, and which in appropriate conditions stimulate or repress transcription. Some transcription factors are active when they are in the form of a monomer. Alternatively, other transcription factors are active in the form of a dimer consisting of two identical proteins or different proteins (heterodimer). Modified forms of transcription factors are intended to refer to transcription factors having a postranslational modification, such as the attachment of a phosphate group. The activity of a transcription factor is frequently modulated by a postranslational modification. For example, certain transcription factors are active only if they are phosphorylated on specific residues. Alternatively, transcription factors can be active in the absence of phosphorylated residues and become inactivated by phosphorylation. A list of known transcription factors and their DNA binding site can be found, e.g., in public databases, e.g., TFMATRIX Transcription Factor Binding Site Profile database.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site.

SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect.

As used herein, the term “specifically hybridizes” or “specifically detects” refers to the ability of a nucleic acid molecule of the invention to hybridize to at least approximately 6, 12, 20, 30, 40, 50, 60, 70, 80, 90, 100, 110, 120, 130 or 140 consecutive nucleotides of either strand of a CD36L1 gene.

“CD36L1” or “CD36L1 receptor” refers to a class B scavenger receptor that has been shown to bind HDL cholesterol and mediate uptake into cells (Acton, S. et al., Science 271:518-520). CD36L1 has also been shown to bind with high affinity to modified proteins (e.g. acetylated LDL, oxidized LDL, maleylated bovine serum albumin) and native LDL (Acton, et al., (1994) J. Biochem. 269:21003-21009). Further, CD36L1 has been shown to bind anionic phospholipids, such as phosphatidylserine and phosphatidylinositol, but not zwitterionic phospholipids, such as phosphatidylcholine, phosphatidylethanolamine and sphingomyelin. Competition studies suggest that anionic phospholipids bind to CD36L1 at a site close to or identical with the sites of native and modified LDL binding and that the interaction may involve polyvalent binding via multiple anionic phospholipid molecules (Rigotti, A., S. Acton and M. Krieger (1995) J. Biochem 270:16221-16224). CD36L1 has also been shown to bind to negatively charged liposomes and apoptotic cells. The human CD36L1 protein is described in Calvo et al. (1993) J. Biol. Chem. 268:18929 and hamster CD36L1 is described in International Patent Application Number WO 96/00288 entitled “Class B1 and C1 Scavenger Receptors” by Acton, S. et al.

The term “CD36L1 therapeutic” refers to various forms of CD36L1 polypeptides, as well as peptidomimetics, nucleic acids, or small molecules, which can modulate at least one activity of a CD36L1 by mimicking or potentiating (agonizing) or inhibiting (antagonizing) the effects of a naturally-occurring CD36L1 polypeptide. A CD36L1 therapeutic which mimics or potentiates the activity of a wild-type CD36L1 polypeptide is a “CD36L1 agonist”. Conversely, a CD36L1 therapeutic which inhibits the activity of a wild-type CD36L1 polypeptide is a “CD36L1 antagonist”. CD36L1 therapeutics can be used to treat diseases which are associated with a specific CD36L1 allele which encodes a protein having an amino acid sequence that differs from that of the wild-type CD36L1 protein.

As used herein, the term “transfection” means the introduction of a nucleic acid, e.g., an expression vector, into a recipient cell by nucleic acid-mediated gene transfer. The term “transduction” is generally used herein when the transfection with a nucleic acid is by viral delivery of the nucleic acid. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the recombinant protein is disrupted.

As used herein, the term “transgene” refers to a nucleic acid sequence which has been genetic-engineered into a cell. Daughter cells deriving from a cell in which a transgene has been introduced are also said to contain the transgene (unless it has been deleted). A transgene can encode, e.g., a polypeptide, or an antisense transcript, partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). Alternatively, a transgene can also be present in an episome. A transgene can include one or more transcriptional regulatory sequence and any other nucleic acid, (e.g. intron), that may be necessary for optimal expression of a selected nucleic acid.

A “transgenic animal” refers to any animal, preferably a non-human animal, e.g. a mammal, bird or an amphibian, in which one or more of the cells of the animal contain heterologous nucleic acid introduced by genetic engineering, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of a protein, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

The term “treatment”, or “treating” as used herein, is defined as the application or administration of a therapeutic agent to a subject, implementation of lifestyle changes (e.g., changes in diet, exercise, or environment), administration of medication, e.g., lipid modulating, e.g., lowering, medication, or application or administration of a therapeutic agent to an isolated tissue or cell line from a subject, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease. The treatments described in the methods of the invention can also be used in combination with a modulator of CD36L1 gene expression or CD36L1 polypeptide activity. “Modulators of CD36L1 gene expression,” as used herein include, for example, CD36L1 nucleic acid molecules, antisense CD36L1 nucleic acid molecules, ribozymes, or a small molecules. “Modulators of CD36L1 polypeptide activity” include, for example, CD36L1-specific antibodies or CD36L1 proteins or polypeptides.

As used herein, the term “vector” refers to a nucleic acid molecule capable of transporting or replicating another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively-linked are referred to herein as “expression vectors”. In general, expression vectors of utility in recombinant DNA techniques are often in the form of “plasmids” which refer generally to circular double stranded DNA circles which, in their vector form are not physically linked to the host chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

Polymorphisms Used in the Methods of the Invention

The nucleic acid molecules of the present invention include specific allelic variants of the CD36L1 gene, which differ from the reference sequence set forth in SEQ ID NO:1 or SEQ ID NO:3, or at least a portion thereof, having a polymorphic region. The preferred nucleic acid molecules of the present invention comprise the EX8 polymorphism and the IVS5 polymorphism of the CD36L1 gene, and those polymorphisms in linkage disequilibrium therewith, which are associated with high TG level or high TG:HDL-C ratio. The invention further comprises isolated nucleic acid molecules complementary to nucleic acid molecules comprising the polymorphisms described in the present invention. Nucleic acid molecules of the present invention can function as probes or primers, e.g., in methods for determining the allelic identity of a CD36L1 polymorphic region. The nucleic acids of the invention can also be used, either in combination with each other or in combination with other SNPs in the CD36L1 gene or other genes, to determine whether a subject is or is not at risk of developing a disease associated with a specific allelic variant of a CD36L1 polymorphic region, e.g., a vascular disease or disorder. The nucleic acids of the invention can further be used to prepare or express CD36L1 polypeptides encoded by specific alleles, such as mutant alleles. Such nucleic acids can be used in gene therapy. Polypeptides encoded by specific CD36L1 alleles, such as mutant CD36L1 polypeptides, can also be used in therapy or for preparing reagents, e.g., antibodies, for detecting CD36L1 proteins encoded by these alleles. Accordingly, such reagents can be used to detect mutant CD36L1 proteins.

As described herein, associations between combinations of allelic variants of the human CD36L1 gene and TG level or TG:HDL-C ratio in women have been identified. The invention is intended to encompass the allelic variants as well as those in linkage disequilibrium which can be identified, e.g., according to the methods described herein.

As described below, one aspect of the invention pertains to isolated nucleic acids comprising an intronic sequence of a CD36L1 gene. In a preferred embodiment, the invention provides an intronic sequence of the genomic DNA sequence encoding a CD36L1 protein, comprising an intronic sequence shown in FIG. 2A-G or set forth in any of SEQ ID NOs.1-121 or complements thereof or homologues thereof. Other preferred nucleic acids of the invention include specific CD36L1 alleles, which differ from the allelic variant having the nucleotide sequence set forth in SEQ ID NO: 1 or SEQ ID NO: 3, or at least a portion thereof having a polymorphic region. Nucleic acids of the invention can function as probes or primers, e.g., in methods for determining the identity of an allelic variant of a CD36L1 polymorphic region. The nucleic acids of the invention can also be used to determine whether a subject is at risk of developing a disease associated with a specific allelic variant of a CD36L1 polymorphic region, e.g, a disease or disorder associated with an aberrant CD36L1 activity. The nucleic acids of the invention can further be used to prepare CD36L1 polypeptides encoded by specific alleles, such as mutant alleles. Such polypeptides can be used in gene therapy. Polypeptides encoded by specific CD36L1 alleles, such as mutant CD36L1 polypeptides, can also be used for preparing reagents, e.g., antibodies, for detecting CD36L1 proteins encoded by these alleles. Accordingly, such reagents can be used to detect mutant CD36L1 proteins.

Certain nucleic acids of the invention comprise an intronic sequence of a CD36L1 gene. The term “CD36L1 intronic sequence” refers to a nucleotide sequence of an intron of a CD36L1 gene. An intronic sequence can be directly adjacent to an exon or located further away from the exons. Preferred nucleic acids of the invention include an intronic sequence of a CD36L1 gene which is adjacent to an exon and comprises at least about 3 consecutive nucleotides, at least about 6 consecutive nucleotides, at least about 9 consecutive nucleotides, at least about 12 consecutive nucleotides, at least about 15 consecutive nucleotides, at least about 18 consecutive nucleotides, or at least about 20 consecutive nucleotides. Isolated nucleic acids which comprise a CD36L1 intronic sequence which is immediately adjacent to an exon and comprises at least about 25 consecutive nucleotides, at least about 30 consecutive nucleotides, at least about 35 consecutive nucleotides, at least about 40 consecutive nucleotides, at least about 50 consecutive nucleotides, or at least about 100 consecutive nucleotides are also within the scope of the invention. Preferred isolated nucleic acids of the invention also include those having a CD36L1 intronic sequence having a nucleotide sequence of at least about 10 nucleotides, at least about 15 nucleotides, at least about 20 nucleotides, at least about 25 nucleotides, at least about 30 nucleotides, at least about 35 nucleotides, at least about 40 nucleotides, at least about 50 nucleotides or at least about 100 nucleotides. Other preferred nucleic acids of the invention can comprise a CD36L1 intronic sequence having less than about 10 nucleotides, provided that the nucleotide sequence is novel. Yet other preferred isolated nucleic acids of the invention include CD36L1 intronic nucleic acid sequences of a CD36L1 intron, having at least about 150 consecutive nucleotides, at least about 200 consecutive nucleotides, at least about 250 consecutive nucleotides, at least about 300 consecutive nucleotides, at least about 350 consecutive nucleotides, at least about 400 consecutive nucleotides, at least about 500 consecutive nucleotides or at least about 1000 consecutive nucleotides

Preferred nucleic acids of the invention comprise a CD36L1 intronic sequence having a nucleotide sequence shown in FIG. 2A-G, and/or in any of SEQ ID Nos. 1-121, complement thereof, reverse complement thereof or homologue thereof. In a preferred embodiment, the invention provides an isolated nucleic acid comprising a CD36L1 intronic which is at least about 70% 75%, 80%, 85%, 90%, 95%, or preferably at least about 98%, and most preferably at least about 99% identical to an intronic nucleotide sequence shown in FIG. 2A-G or set forth in any of SEQ ID NOS.1-121 or a complement thereof. In fact, as described herein, several alleles of human CD36L1 genes have been identified. The invention is intended to encompass all of these alleles and CD36L1 alleles not yet identified, which can be identified, e.g, according to the methods described herein.

The invention also provides isolated nucleic acids comprising at least one polymorphic region of a CD36L1 gene having a nucleotide sequence which differs from the nucleotide sequence set forth in SEQ ID NO: 1 or SEQ ID NO: 3. Preferred nucleic acids have a polymorphic region located in an exon of a CD36L1 gene, such as exon 8. Accordingly, preferred nucleic acids of the invention comprise a thymidine at position 41 of exon 8 (as set forth in SEQ ID NO: 65). Preferred nucleic acids can also have a polymorphic region in an intron, e.g., intron 5 or 10. For example, the invention provides nucleic acids having a polymorphic nucleotide at position 54 of intron 5. In a preferred embodiment, the nucleic acid has a thymidine at position 54 of intron 5 (as set forth in SEQ ID NO: 66). The nucleic acids can be genomic DNA, cDNA, or RNA (in which case, the nucleic acid has a uridine at position 54 of intron 5).

Also within the scope of the invention are isolated nucleic acids which encode a CD36L1 protein, such as a CD36L1 protein having an amino acid sequence which differs from the amino acid sequence set forth in SEQ ID NOs 2 and 4.

Preferred nucleic acids of the invention are from vertebrate genes encoding CD36L1 proteins. Particularly preferred vertebrate nucleic acids are mammalian nucleic acids. A particularly preferred nucleic acid of the invention is a human nucleic acid, such as a nucleic acid comprising a CD36L1 intronic sequence shown in FIG. 2A-G or set forth in any of SEQ ID NOS. 1-121 or complement thereof or an allele comprising a nucleotide sequence set forth in SEQ ID NO: 65 or SEQ ID NO: 97.

Another aspect of the invention provides a nucleic acid which hybridizes under appropriate stringency to a CD36L1 intronic sequence having a nucleotide sequence shown in introns shown in FIG. 2A-G or in intronic sequences set forth in any of SEQ ID Nos. 1-121 or complement thereof. As used herein, the term “hybridizes” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions vary according to the length of the involved nucleotide sequence but are known to those skilled in the art and can be found or determined based on teachings in Current Protocols in Molecular Biology, Ausubel et al., eds., John Wiley & Sons, Inc. (1995), sections 2, 4 and 6. Additional stringent conditions and formulas for determining such conditions can be found in Molecular Cloning. A Laboratory Manual, Sambrook et al., Cold Spring Harbor Press, Cold Spring Harbor, N.Y. (1989), chapters 7, 9 and 11. A preferred, non-limiting example of stringent hybridization conditions for hybrids that are at least basepairs in length includes hybridization in 4×sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions for such hybrids includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions for such hybrids includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete.

The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In a preferred embodiment, a nucleic acid of the present invention will bind to at least about 20, preferably at least about 25, more preferably at least about 30 and most preferably at least about 50 consecutive nucleotides of a sequence shown in FIG. 2A-G or set forth in any of SEQ ID Nos.1-121 under moderately stringent conditions, for example at about 2.0×SSC and about 40° C. Even more preferred nucleic acids of the invention are capable of hybridizing under stringent conditions to an intronic sequence of at least about 20, 30, 40, or at least about 50 nucleotides as shown in FIG. 2A-G or as set forth in an intronic sequence of any of SEQ ID Nos.1-121.

Hybridization, as described above, can be used to isolate nucleic acids comprising a CD36L1 intron or portion thereof from various animal species. A comparison of these nucleic acids should be indicative of intronic sequences which may have a regulatory or other function, since these regions are expected to be conserved among various species. Hybridization can also be used to isolate CD36L1 alleles.

The nucleic acid of the invention can be single stranded DNA (e.g, an oligonucleotide), double stranded DNA (e.g., double stranded oligonucleotide) or RNA. Preferred nucleic acids of the invention can be used as probes or primers. Primers of the invention refer to nucleic acids which hybridize to a nucleic acid sequence which is adjacent to the region of interest or which covers the region of interest and is extended. A primer can be used alone in a detection method, or a primer can be used together with at least one other primer or probe in a detection method. Primers can also be used to amplify at least a portion of a nucleic acid. Probes of the invention refer to nucleic acids which hybridize to the region of interest and which are not further extended. For example, a probe is a nucleic acid which hybridizes to a polymorphic region of a CD36L1 gene, and which by hybridization or absence of hybridization to the DNA of a subject will be indicative of the identity of the allelic variant of the polymorphic region of the CD36L1 gene.

Numerous procedures for determining the nucleotide sequence of a nucleic acid, or for determining the presence of mutations in nucleic acids include a nucleic acid amplification step, which can be carried out by, e.g., polymerase chain reaction (PCR). Accordingly, in one embodiment, the invention provides primers for amplifying portions of a CD36L1 gene, such as portions of exons and/or portions of introns. In a preferred embodiment, the exons and/or sequences adjacent to the exons of the human CD36L1 gene will be amplified to, e.g, detect which allelic variant of a polymorphic region is present in the CD36L1 gene of a subject. Preferred primers comprise a nucleotide sequence complementary to a CD36L1 intronic sequence or a specific allelic variant of a CD36L1 polymorphic region and of sufficient length to selectively hybridize with a CD36L1 gene. In a preferred embodiment, the primer, e.g., a substantially purified oligonucleotide, comprises a region having a nucleotide sequence which hybridizes under stringent conditions to about 6, 8, 10, or 12, preferably 25, 30, 40, 50, or 75 consecutive nucleotides of a CD36L1 gene. In an even more preferred embodiment, the primer is capable of hybridizing to a CD36L1 intron and has a nucleotide sequence of an intronic sequence shown in FIG. 2A-G or set forth in any of SEQ ID Nos. 1-121, complements thereof, allelic variants thereof, or complements of allelic variants thereof. For example, primers comprising a nucleotide sequence of at least about 15 consecutive nucleotides, at least about 20 nucleotides or having from about 15 to about 25 nucleotides shown in FIG. 2A-G or set forth in any of SEQ ID NOS. 1-121 or complement thereof are provided by the invention. Primers having a sequence of more than about 25 nucleotides are also within the scope of the invention. Preferred primers of the invention are primers that can be used in PCR for amplifying each of the exons of a CD36L1 gene. Even more preferred primers of the invention have the nucleotide sequence set forth in any of SEQ ID Nos. 41-64 and 89-94.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 150 to about 350 nucleotides apart.

For amplifying at least a portion of a nucleic acid, a forward primer (i.e., 5′ primer) and a reverse primer (i e., 3′ primer) will preferably be used. Forward and reverse primers hybridize to complementary strands of a double stranded nucleic acid, such that upon extension from each primer, a double stranded nucleic acid is amplified. A forward primer can be a primer having a nucleotide sequence or a portion of the nucleotide sequence shown in FIG. 2A-G or in SEQ ID Nos. 1-40, 65, 66, and 95-97. A reverse primer can be a primer having a nucleotide sequence or a portion of the nucleotide sequence that is complementary to a nucleotide sequence shown in FIG. 2A-G or in SEQ ID Nos. 1-40, 65, 66, and 95-97. Preferred forward primers comprise a nucleotide sequence set forth in SEQ ID Nos. 41, 43, 45, 47, 49, 51, 53, 55, 57, 59, 61, 63, and 85. Preferred reverse primers comprise a nucleotide sequence set forth in SEQ ID Nos. 42, 44, 46, 48, 50, 52, 54, 56, 58, 60, 62, 64, and 86.

Yet other preferred primers of the invention are nucleic acids which are capable of selectively hybridizing to an allelic variant of a polymorphic region of a CD36L1 gene. Thus, such primers can be specific for a CD36L1 gene sequence, so long as they have a nucleotide sequence which is capable of hybridizing to a CD36L1 gene. Preferred primers are capable of specifically hybridizing to an allelic variant in which nucleotide 146 of exon 1 of human CD36L1 is an adenine, e.g., a nucleic acid having SEQ ID NO: 95; an allelic variant in which nucleotide 119 of exon 3 is an adenine, e.g., a nucleic acid having SEQ ID NO: 96; or an allelic variant in which nucleotide 41 of exon 8 of human CD36L1 is a thymidine, e.g., a nucleic acid having SEQ ID NO: 65. Other preferred primers are capable of specifically hybridizing to an allelic variant in which nucleotide 54 of intron 5 is a thymidine, e.g., a nucleic acid having SEQ ID NO: 66 or nucleotide −41 of intron 10 is a guanine, e.g., a nucleic acid having SEQ ID NO: 97. Such primers can be used, e.g., in sequence specific oligonucleotide priming as described further herein.

The CD36L1 nucleic acids of the invention can also be used as probes, e.g., in therapeutic and diagnostic assays. For instance, the present invention provides a probe comprising a substantially purified oligonucleotide, which oligonucleotide comprises a region having a nucleotide sequence that hybridizes under stringent conditions to at least approximately 6, 8, 10 or 12, preferably about 25, 30, 40, 50 or 75 consecutive nucleotides of a CD36L1 gene. In one embodiment, the probes preferably hybridize to an intron of a CD36L1 gene, having an intronic nucleotide sequence shown in FIG. 2A-G or set forth in any of SEQ ID Nos. 1-121, allelic variants thereof, complements thereof or complements of allelic variants thereof. In another embodiment, the probes are capable of hybridizing to a nucleotide sequence encompassing an intron/exon border of a CD36L1 gene.

Other preferred probes of the invention are capable of hybridizing specifically to a region of a CD36L1 gene which is polymorphic. In an even more preferred embodiment of the invention, the probes are capable of hybridizing specifically to one allelic variant of a CD36L1 gene having a nucleotide sequence which differs from the nucleotide sequence set forth in SEQ ID NO: 1 or 3. Such probes can then be used to specifically detect which allelic variant of a polymorphic region of a CD36L1 gene is present in a subject. The polymorphic region can be located in the promoter, exon, or intron sequences of a CD36L1 gene.

Other preferred probes of the invention are capable of hybridizing specifically to a region overlapping nucleotide 41 of exon 8 of the human CD36L1 gene. In one embodiment, the probe overlapping nucleotide 41 of exon 8 is capable of hybridizing specifically to a nucleotide sequence wherein nucleotide 41 is a thymidine (as shown in SEQ ID NO: 65). Examples of such probes include a probe having the nucleotide sequence 5′ AACCGGGTCAGCGTTGAGGA 3′ (SEQ ID NO: 67); 5′ TGCCAGAACCGGGTCAGCGTTGAGGAAGTGA 3′ (SEQ ID NO: 68); and probes having the complement of these nucleotide sequences, i.e., 5′ TCCTCAACGCTGACCCGGTT 3′ (SEQ ID NO: 69); 5′ TCACTTCCTCAACGCTGACCCGGTTCTGGCA 3′ (SEQ ID NO: 70). The bold nucleotides represents the location of the nucleotide polymorphism. In another embodiment, the probe overlapping nucleotide 41 of exon 8 is capable of specifically hybridizing to a nucleotide sequence wherein nucleotide 41 is a cytidine (as shown in FIG. 2A-G and set forth in SEQ ID NO: 12). Examples of such probes include a probe having the nucleotide sequence 5′ AACCGGGTCGGCGTTGATGA 3′ (SEQ ID NO: 71); TGCCAGAACCGGGTCGGCGTTGATGAAGTGA 3′ (SEQ ID NO: 72) and probes having the complement of these nucleotide sequences, i.e., 5′ TCATCAACGCCGACCCGGTT 3′ (SEQ ID NO: 73); 5′ TCACTTCATCAACGCCGACCCGGTTCTGGCA 3′ (SEQ ID NO: 74).

Yet other preferred probes of the invention are capable of hybridizing specifically to a region overlapping nucleotide 54 of intron 5 of the human CD36L1 gene. In one embodiment, the probe overlapping nucleotide 54 of intron 5 is capable of hybridizing specifically to a nucleotide sequence wherein nucleotide 54 is a cytidine (as shown in FIG. 2A-G and set forth in SEQ ID NOS. 9 and 26). Examples of such probes include a probe having the nucleotide sequence 5′ AGCCATGGCCGGGCCCACCCT 3′ (SEQ ID NO: 75); 5′ CGAGCAGCCATGGCCGGGCCCACCCTCCCCT 3′ (SEQ ID NO: 76); and probes having the complement of these nucleotide sequences, i.e., 5′ AGGGTGGGCCCGGCCATGGCT 3′ (SEQ ID NO: 77); 5′ AGGGGAGGGTGGGCCCGGCCATGGCTGCTCG 3′ (SEQ ID NO: 78). In another embodiment, the probe overlapping nucleotide 54 of intron 5 is capable of specifically hybridizing to a nucleotide sequence wherein nucleotide 54 is a thymidine (as shown in SEQ ID NO: 66). Examples of such probes include a probe having the nucleotide sequence 5′ AGCCATGGCCAGGCCCACCCT 3′ (SEQ ID NO: 79); 5′ CGAGCAGCCATGGCCAGGCCCACCCTCCCCT 3′ (SEQ ID NO: 80); and probes having the complement of these nucleotide sequences, i.e., 5′ AGGGTGGGCCTGGCCATGGCT 3′ (SEQ ID NO: 81); 5′ AGGGGAGGGTGGGCCTGGCCATGGCTGCTCG 3′ (SEQ ID NO: 82).

Particularly, preferred probes of the invention have a number of nucleotides sufficient to allow specific hybridization to the target nucleotide sequence. Where the target nucleotide sequence is present in a large fragment of DNA, such as a genomic DNA fragment of several tens or hundreds of kilobases, the size of the probe may have to be longer to provide sufficiently specific hybridization, as compared to a probe which is used to detect a target sequence which is present in a shorter fragment of DNA. For example, in some diagnostic methods, a portion of a CD36L1 gene may first be amplified and thus isolated from the rest of the chromosomal DNA and then hybridized to a probe. In such a situation, a shorter probe will likely provide sufficient specificity of hybridization. For example, a probe having a nucleotide sequence of about 10 nucleotides may be sufficient.

In preferred embodiments, the probe or primer further comprises a label attached thereto, which, e.g., is capable of being detected, e.g. the label group is selected from amongst radioisotopes, fluorescent compounds, enzymes, and enzyme co-factors.

In a preferred embodiment of the invention, the isolated nucleic acid, which is used, e.g., as a probe or a primer, is modified, such as to become more stable. Exemplary nucleic acid molecules which are modified include phosphoramidate, phosphothioate and methylphosphonate analogs of DNA (see also U.S. Pat. Nos. 5,176,996; 5,264,564; and 5,256,775).

The nucleic acids of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the nucleic acid of the invention may be conjugated to another molecule, e.g., a peptide, hybridization triggered cross-linking agent, transport agent, hybridization-triggered cleavage agent, etc.

The isolated nucleic acid comprising a CD36L1 intronic sequence may comprise at least one modified base moiety which is selected from the group including but not limited to 5-fluorouracil, 5-bromouracil, 5-chlorouracil, 5-iodouracil, hypoxanthine, xantine, 4-acetylcytidine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytidine, 5-methylcytidine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytidine, 5-methyl-2-thiouracil, 2-thiouracil, 4-thiouracil, 5-methyluracil, uracil-5- oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The isolated nucleic acid may also comprise at least one modified sugar moiety selected from the group including but not limited to arabinose, 2-fluoroarabinose, xylulose, and hexose.

In yet another embodiment, the nucleic acid comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the nucleic acid is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBSLett. 215:327-330).

Any nucleic acid fragment of the invention can be prepared according to methods well known in the art and described, e.g., in Sambrook, J. Fritsch, E. F., and Maniatis, T. (1989) Molecular Cloning: A Laboratory Manual, Cold Spring Harbor Laboratory Press, Cold Spring Harbor, N.Y. For example, discrete fragments of the DNA can be prepared and cloned using restriction enzymes. Alternatively, discrete fragments can be prepared using the Polymerase Chain Reaction (PCR) using primers having an appropriate sequence.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The invention also provides vectors and plasmids containing the nucleic acids of the invention. For example, in one embodiment, the invention provides a vector comprising at least a portion of a CD36L1 gene comprising a polymorphic region and/or intronic sequence. Thus, the invention provides vectors for expressing at least a portion of the newly identified allelic variants of the human CD36L1 gene, as well as other allelic variants, having a nucleotide sequence which is different from the nucleotide sequence disclosed in Calvo and Vega, supra. The allelic variants can be expressed in eukaryotic cells, e.g., cells of a subject, or in prokaryotic cells.

In one embodiment, the vector comprising at least a portion of a CD36L1 allele is introduced into a host cell, such that a protein encoded by the allele is synthesized. The CD36L1 protein produced can be used, e.g., for the production of antibodies, which can be used, e.g., in methods for detecting mutant forms of CD36L1. Alternatively, the vector can be used for gene therapy, and be, e.g., introduced into a subject to produce CD36L1 protein. Host cells comprising a vector having at least a portion of a CD36L1 gene are also within the scope of the invention.

Polypeptides Used in the Methods of the Invention

The present invention describes isolated CD36L1 polypeptides, such as CD36L1 polypeptides which are encoded by specific allelic variants of CD36LI, including those identified herein.

In one embodiment, the CD36L1 polypeptides are isolated from, or otherwise substantially free of other cellular proteins. The term “substantially free of other cellular proteins” (also referred to herein as “contaminating proteins”) or “substantially pure or purified preparations” are defined as encompassing preparations of CD36L1 polypeptides having less than about 20% (by dry weight) contaminating protein, and preferably having less than about 5% contaminating protein. It will be appreciated that functional forms of the subject polypeptides can be prepared, for the first time, as purified preparations by using a cloned gene as described herein.

Preferred CD36L1 proteins of the invention have an amino acid sequence which is at least about 60%, 70%, 80%, 85%, 90%, or 95% identical or homologous to the amino acid sequence of SEQ ID NO:2 or 4. Even more preferred CD36L1 proteins comprise an amino acid sequence which is at least about 95%, 96%, 97%, 98%, or 99% homologous or identical to the amino acid sequence of SEQ ID NO:2 or 4. Such proteins can be recombinant proteins, and can be, e.g., produced in vitro from nucleic acids comprising a specific allele of a CD36L1 polymorphic region. For example, recombinant polypeptides preferred by the present invention can be encoded by a nucleic acid which comprises a sequence which is at least 85% homologous and more preferably 90% homologous and most preferably 95% homologous with a nucleotide sequence set forth in SEQ ID NO:1 or 3 and comprises an allele of a polymorphic region that differs from that set forth in SEQ ID NO:1 or 3. Polypeptides which are encoded by a nucleic acid comprising a sequence that is at least about 98-99% homologous with the sequence of SEQ ID NO:1 or 3 and comprises an allele of a polymorphic region that differs from that set forth in SEQ ID NO:1 or 3 are also within the scope of the invention.

In a preferred embodiment, a CD36L1 protein of the present invention is a mammalian CD36L1 protein. In an even more preferred embodiment, the CD36L1 protein is a human protein.

The invention also provides peptides that preferably are capable of functioning in one of either role of an agonist or antagonist of at least one biological activity of a wild-type (“normal”) CD36L1 protein of the appended sequence listing. The term “evolutionarily related to,” with respect to amino acid sequences of CD36L1 proteins, refers to both polypeptides having amino acid sequences found in human populations, and also to artificially produced mutational variants of human CD36L1 polypeptides which are derived, for example, by combinatorial mutagenesis.

Full length proteins or fragments corresponding to one or more particular motifs and/or domains or to arbitrary sizes, for example, at least 5, 10, 25, 50, 75 and 100, amino acids in length of CD36L1 protein are within the scope of the present invention.

Isolated CD36L1 peptides or polypeptides can be obtained by screening peptides recombinantly produced from the corresponding fragment of the nucleic acid encoding such peptides. In addition, such peptides and polypeptides can be chemically synthesized using techniques known in the art such as conventional Merrifield solid phase f-Moc or t-Boc chemistry. For example, a CD36L1 peptide or polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptides or polypeptides which can function as either agonists or antagonists of a wild-type (e.g., “normal”) CD36L1 protein.

In general, peptides and polypeptides referred to herein as having an activity (e.g., are “bioactive”) of a CD36L1 protein are defined as peptides and polypeptides which mimic or antagonize all or a portion of the biological/biochemical activities of a CD36L1 protein having SEQ ID NO:2 or 4, such as the ability to bind ligands. Other biological activities of the subject CD36L1 proteins are described herein or will be reasonably apparent to those skilled in the art. According to the present invention, a peptide or polypeptide has biological activity if it is a specific agonist or antagonist of a naturally-occurring form of a CD36L1 protein.

Assays for determining whether a CD36L1 protein or variant thereof, has one or more biological activities are well known in the art.

Other preferred proteins of the invention are those encoded by the nucleic acids set forth in the section pertaining to nucleic acids of the invention. In particular, the invention provides fusion proteins, e.g., CD36L1-immunoglobulin fusion proteins. Such fusion proteins can provide, e.g., enhanced stability and solubility of CD36L1 proteins and may thus be useful in therapy. Fusion proteins can also be used to produce an immunogenic fragment of a CD36L1 protein. For example, the VP6 capsid protein of rotavirus can be used as an immunologic carrier protein for portions of the CD36L1 polypeptide, either in the monomeric form or in the form of a viral particle. The nucleic acid sequences corresponding to the portion of a subject CD36L1 protein to which antibodies are to be raised can be incorporated into a fusion gene construct which includes coding sequences for a late vaccinia virus structural protein to produce a set of recombinant viruses expressing fusion proteins comprising CD36L1 epitopes as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of a CD36L1 protein and the poliovirus capsid protein can be created to enhance immunogenicity of the set of polypeptide antigens (see, for example, EP Publication No: 0259149; and Evans et al. (1989) Nature 339:385; Huang et al. (1988) J. Virol. 62:3855; and Schlienger et al. (1992) J. Virol. 66:2).

The Multiple antigen peptide system for peptide-based immunization can also be utilized to generate an immunogen, wherein a desired portion of a CD36L1 polypeptide is obtained directly from organo-chemical synthesis of the peptide onto an oligomeric branching lysine core (see, for example, Posnett et al. (1988) JBC 263:1719 and Nardelli et al. (1992) J. Immunol. 148:914). Antigenic determinants of CD36L1 proteins can also be expressed and presented by bacterial cells.

Fusion proteins can also facilitate the expression of proteins including the CD36L1 polypeptides of the present invention. For example, CD36L1 polypeptides can be generated as glutathione-S-transferase (GST-fusion) proteins. Such GST-fusion proteins can be easily purified, as for example by the use of glutathione-derivatized matrices (see, for example, Current Protocols in Molecular Biology, eds. Ausubel et al. (N.Y.: John Wiley & Sons, 1991)) and used subsequently to yield purified CD36L1 polypeptides.

The present invention further pertains to methods of producing the subject CD36L1 polypeptides. For example, a host cell transfected with a nucleic acid vector directing expression of a nucleotide sequence encoding the subject polypeptides can be cultured under appropriate conditions to allow expression of the peptide to occur. Suitable media for cell culture are well known in the art. The recombinant CD36L1 polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant CD36L1 polypeptide is a fusion protein containing a domain which facilitates its purification, such as GST fusion protein.

Moreover, it will be generally appreciated that, under certain circumstances, it may be advantageous to provide homologs of one of the subject CD36L1 polypeptides which function in a limited capacity as one of either a CD36L1 agonist (mimetic) or a CD36L1 antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of CD36L1 proteins.

Homologs of each of the subject CD36L1 proteins can be generated by mutagenesis, such as by discrete point mutation(s), and/or by truncation. For instance, mutation can give rise to homologs which retain substantially the same, or merely a subset, of the biological activity of the CD36L1 polypeptide from which it was derived. Alternatively, antagonistic forms of the protein can be generated which are able to inhibit the function of the naturally occurring form of the protein, such as by competitively binding to a CD36L1 receptor.

The recombinant CD36L1 polypeptides of the present invention also include homologs of CD36L1 polypeptides which differ from the CD36L1 protein having SEQ ID NO:2 or 4, such as versions of the protein which are resistant to proteolytic cleavage, as for example, due to mutations which alter ubiquitination or other enzymatic targeting associated with the protein.

CD36L1 polypeptides may also be chemically modified to create CD36L1 derivatives by forming covalent or aggregate conjugates with other chemical moieties, such as glycosyl groups, lipids, phosphate, acetyl groups and the like. Covalent derivatives of CD36L1 proteins can be prepared by linking the chemical moieties to functional groups on amino acid side-chains of the protein or at the N-terminus or at the C-terminus of the polypeptide.

Modification of the structure of the subject CD36L1 polypeptides can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally-occurring form of the protein, or to produce specific antagonists thereof, are considered functional equivalents of the CD36L1 polypeptides described in more detail herein. Such modified peptides can be produced, for instance, by amino acid substitution, deletion, or addition. The substitutional variant may be a substituted conserved amino acid or a substituted non-conserved amino acid.

For example, it is reasonable to expect that an isolated replacement of a leucine with an isoleucine or valine, an aspartate with a glutamate, a threonine with a serine, or a similar replacement of an amino acid with a structurally related amino acid (i.e., isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic =aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur -containing=cysteine and methionine. (see, for example, Biochemistry, 2^{nd} ed., Ed. by L. Stryer, WH Freeman and Co.: 1981). Whether a change in the amino acid sequence of a peptide results in a functional CD36L1 homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

Methods

The invention further provides predictive medicine methods, which are based, at least in part, on the discovery of CD36L1 polymorphic regions which are associated with specific physiological states and/or diseases or disorders, e.g., abnormal lipid levels, e.g., abnormally high TG levels and/or abnormally high TG:HDL-C ratios. Abnormally high TG levels and abnormally high TG:HDL-C ratios are risk factors associated with vascular diseases or disorders such as CAD and MI and metabolic diseases or disorders such as diabetes or obesity. These methods can be used alone, or in combination with other predictive medicine methods, including the identification and analysis of known risk factors associated with abnormal lipid levels, e.g., abnormally high TG levels and/or abnormally high TG:HDL-C ratios, vascular diseases or disorders, and/or metabolic diseases or disorders, e.g., phenotypic factors and family history.

For example, information obtained using the diagnostic assays described herein (in combination with each other or in combination with information of another genetic defect which contributes to the same disorder, e.g., abnormal lipid levels, e.g., abnormally high TG levels and/or abnormally high TG:HDL-C ratios) is useful for diagnosing or confirming that a subject has an allele of a polymorphic region which is associated with a particular disease or disorder, e.g., abnormal lipid levels, e.g., abnormally high TG levels and/or abnormally high TG:HDL-C ratios, or a combination of alleles which are associated with a particular disease or disorder, e.g., a subject who is heterozygous (CT) at IVS5 and heterozygous (CT) at EX8, or the complement thereof, or homozygous (TT) at IVS5 and heterozygous (CT) at EX8, or the complements thereof. Moreover, the information obtained using the diagnostic assays described herein, in combination with each other or in combination with information of another genetic defect which contributes to the same disease, e.g., abnormal lipid levels, e.g., abnormally high TG levels and/or abnormally high TG:HDL-C ratios, can be used to predict whether or not a subject will benefit from further diagnostic evaluation for abnormal lipid levels, e.g., abnormally high TG levels and/or abnormally high TG:HDL-C ratios, a vascular disease or disorder, or a metabolic disease or disorder. Such further diagnostic evaluation includes, but is not limited to, cardiovascular imaging, such as angiography, cardiac ultrasound, coronary angiogram, magnetic resonance imagery, nuclear imaging, CT scan, myocardial perfusion imagery, or electrocardiogram, genetic analysis, e.g., identification of additional polymorphisms e.g., which contribute to the same disease, familial health history analysis, lifestyle analysis, or exercise stress tests, either alone or in combination. Furthermore, the diagnostic information obtained using the diagnostic assays described herein (in combination with each other or in combination with information of another genetic defect which contributes to the same disease, e.g., abnormal lipid levels, e.g., abnormally high TG level or an abnormally high TG:HDL-C ratio, or a disease or disorder associated with abnormal lipid levels, e.g., a vascular or metabolic disease or disorder), may be used to identify which subject will benefit from a particular clinical course of therapy useful for preventing, treating, ameliorating, or prolonging onset of abnormal lipid levels, a vascular disease or disorder, or a metabolic disease or disorder in the particular subject. Clinical courses of therapy include, but are not limited to, administration of medication, e.g., lipid lowering medication, change in lifestyle, e.g., diet or exercise, non-surgical intervention, surgical procedures such as percutaneous transluminal coronary angioplasty, laser angioplasty, implantation of a stent, coronary bypass grafting, implantation of a defibrillator, implantation of a pacemaker, and any combination thereof, and use of surgical and non-surgical medical devices used in the treatment of vascular disease, such as, for example, a defibrillator, a stent, a device used in coronary revascularization, a pacemaker, and any combination thereof. Medical devices, medication, or lifestyle changes, as described above, may also be used in combination with a modulator of CD36L1 gene expression or CD36L1 polypeptide activity.

Alternatively, the information, in combination with each other, or, preferably, in combination with information of another genetic defect which contributes to the same disease, e.g., abnormal lipid levels, e.g., abnormally high TG level or an abnormally high TG:HDL-C ratio, or a disease or disorder associated with abnormal lipid levels, e.g., a vascular or metabolic disease or disorder, can be used prognostically for predicting whether a non-symptomatic subject is likely to develop a disease or condition which is associated with one or more specific alleles of CD36L1 polymorphic regions in a subject. The information may also be used to predict the response of a female subject to HRT.

Based on the prognostic information, a health care provider can recommend a particular further diagnostic evaluation which will benefit the subject, or a particular clinical course of therapy, as described above.

In addition, knowledge of the identity of one or more particular CD36L1 alleles in a subject (the CD36L1 genetic profile), preferably, the allele at nucleotide residue 41 of exon 8 in combination with the allele at nucleotide residue 54 of intron 5, allows customization of further diagnostic evaluation and/or a clinical course of therapy for a particular disease. For example, a subject's CD36L1 genetic profile or the genetic profile of a disease or disorder associated with a specific allele of a CD36L1 polymorphic region, e.g., abnormal lipid levels, e.g., abnormally high TG levels and/or abnormally high TG:HDL-C ratios, can enable a health care provider: 1) to more efficiently and cost-effectively identify means for further diagnostic evaluation, including, but not limited to, further genetic analysis, familial health history analysis, or use of vascular imaging devices or procedures; 2) to more effectively prescribe a drug that will address the molecular basis of the disease or condition; 3) to more efficiently and cost-effectively identify an appropriate clinical course of therapy, including, but not limited to, lifestyle changes, medications, surgical or non-surgical medical devices, surgical or non-surgical intervention or procedures, or any combination thereof; and 4) to better determine the appropriate dosage of a particular drug or duration of a particular course of clinical therapy. For example, the expression level of CD36L1 proteins, alone or in conjunction with the expression level of other genes known to contribute to the same disease, can be measured in many subjects at various stages of the disease to generate a transcriptional or expression profile of the disease. Expression patterns of individual subjects can then be compared to the expression profile of the disease to determine the appropriate drug, dose to administer to the subject, or course of clinical therapy.

The ability to target populations expected to show the highest clinical benefit, based on the CD36L1 or disease genetic profile, can enable: 1) the repositioning of marketed drugs, medical devices and surgical procedures for use in treating, preventing, or ameliorating vascular diseases or disorders, or diagnostics, such as vascular imaging devices or procedures, with disappointing market results; 2) the rescue of drug candidates whose clinical development has been discontinued as a result of safety or efficacy limitations, which are subject subgroup-specific; 3) an accelerated and less costly development for drug candidates and more optimal drug labeling (e.g., since the use of CD36L1 as a marker is useful for optimizing effective dose); and 4) an accelerated, less costly, and more effective selection of a particular course of clinical therapy suited to a particular subject.

These and other methods are described in further detail in the following sections.

A. Prognostic and Diagnostic Assays

The present methods provide means for determining if a subject has or is or is not at risk of developing a disease, condition or disorder that is associated a specific CD36L1 allele or combinations thereof, e.g., abnormal lipid levels, e.g., abnormally high TG levels and/or abnormally high TG:HDL-C ratios or a vascular disease or disorder or a metabolic disease or disorder associated with abnormal lipid levels. The present methods also provide means for determining if a subject, e.g., a female subject, e.g., a postmenopausal female subject, is at risk for developing abnormal lipid levels and/or diseases or disorders associated with abnormal lipid levels, e.g., CAD or MI, in response to treatment with HRT.

The present invention provides methods for determining the molecular structure of a CD36L1 gene, such as a human CD36L1 gene, or a portion thereof. In one embodiment, determining the molecular structure of at least a portion of a CD36L1 gene comprises determining the identity of the allelic variant of at least one polymorphic region of a CD36L1 gene (determining the genotype at the EX8 polymorphism and the IVS5 polymorphism). A polymorphic region of a CD36L1 gene can be located in an exon, an intron, at an intron/exon border, or in the 5′ upstream regulatory element of the CD36L1 gene.

The invention provides methods for determining whether a subject has or is at risk of developing, a disease or disorder associated with a specific allelic variant of a polymorphic region of a CD36L1 gene. Such diseases can be associated with aberrant CD36L1 activity.

Analysis of one or more CD36L1 polymorphic regions in a subject can be useful for predicting whether a subject has or is likely to develop abnormal lipid levels, e.g., abnormally high TG level or an abnormally high TG:HDL-C ratio, or a disease or disorder associated with abnormal lipid levels, e.g., a vascular disease or disorder, e.g., CAD or MI, or a metabolic disease or disorder e.g., obesity or diabetes.

In preferred embodiments, the methods of the invention can be characterized as comprising detecting, in a sample of cells from the subject, the presence or absence of a specific allelic variant of one or more polymorphic regions of a CD36L1 gene. The allelic differences can be: (i) a difference in the identity of at least one nucleotide or (ii) a difference in the number of nucleotides, which difference can be a single nucleotide or several nucleotides. The invention also provides methods for detecting differences in a CD36L1 gene such as chromosomal rearrangements, e.g., chromosomal dislocation. The invention can also be used in prenatal diagnostics.

A preferred detection method is allele specific hybridization using probes overlapping the polymorphic site and having about 5, 10, 20, 25, or 30 nucleotides around the polymorphic region. In a preferred embodiment of the invention, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism in the 5′ upstream regulatory element can be determined in a single hybridization experiment.

In other detection methods, it is necessary to first amplify at least a portion of a CD36L1 gene prior to identifying the allelic variant. Amplification can be performed, e.g., by PCR and/or LCR (see Wu and Wallace, (1989) Genomics 4:560), according to methods known in the art. In one embodiment, genomic DNA of a cell is exposed to two PCR primers and amplification for a number of cycles sufficient to produce the required amount of amplified DNA. In preferred embodiments, the primers are located between 150 and 350 base pairs apart.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al., 1988, Bio/Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of a CD36L1 gene and detect allelic variants, e.g., mutations, by comparing the sequence of the sample sequence with the corresponding reference (control) sequence. Exemplary sequencing reactions include those based on techniques developed by Maxam and Gilbert (Proc. Natl Acad Sci USA (1977) 74:560) or Sanger (Sanger et al. (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled “DNA Sequencing by Mass Spectrometry Via Exonuclease Degradation” by H. Köster), and U.S. Pat. No. 5,605,798 and International Patent Application No. PCT/US96/03651 entitled DNA Diagnostics Based on Mass Spectrometry by H. Köster; Cohen et al (1996) Adv Chromatogr 36:127-162; and Griffin et al (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

In some cases, the presence of a specific allele of a CD36L1 gene in DNA from a subject can be shown by restriction enzyme analysis. For example, a specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of a CD36L1 allelic variant with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al. (1988) Proc. Natl Acad Sci USA 85:4397; Saleeba et al (1992) Methods Enzymol. 217:286-295. In a preferred embodiment, the control or sample nucleic acid is labeled for detection.

In another embodiment, an allelic variant can be identified by denaturing high-performance liquid chromatography (DHPLC) (Oefner and Underhill, (1995) Am. J. Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility is used to identify the type of CD36L1 allelic variant. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl Acad. Sci USA 86:6230; and Wallace et al. (1979) Nucl. Acids Res. 6:3543). Such allele specific oligonucleotide hybridization techniques may be used for the simultaneous detection of several nucleotide changes in different polymorphic regions of CD36L1. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner(1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al., (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., (1990) Proc. Natl. Acad. Sci. (U.S.A.) 87:8923-8927. In this method, PCR is used to achieve the exponential amplification of target DNA, which is then detected using OLA.

Several techniques based on this OLA method have been developed and can be used to detect specific allelic variants of a polymorphic region of a CD36L1 gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide polymorphisms in a CD36L1 gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site (Cohen, D. et al. (French Patent 2,650,840; PCT Application No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Application No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. W091/02087) the method of Goelet, P. et al. is preferably a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

For determining the identity of the allelic variant of a polymorphic region located in the coding region of a CD36L1 gene, yet other methods than those described above can be used. For example, identification of an allelic variant which encodes a mutated CD36L1 protein can be performed by using an antibody specifically recognizing the mutant protein in, e.g., immunohistochemistry or immunoprecipitation. Antibodies to wild-type CD36L1 or mutated forms of CD36L1 proteins can be prepared according to methods known in the art.

Alternatively, one can also measure an activity of a CD36L1 protein, such as binding to a CD36L1 ligand. Binding assays are known in the art and involve, e.g., obtaining cells from a subject, and performing binding experiments with a labeled lipid, to determine whether binding to the mutated form of the protein differs from binding to the wild-type of the protein.

Antibodies directed against reference or mutant CD36L1 polypeptides or allelic variant thereof, which are discussed above, may also be used in disease diagnostics and prognostics. Such diagnostic methods, may be used to detect abnormalities in the level of CD36L1 polypeptide expression, or abnormalities in the structure and/or tissue, cellular, or subcellular location of a CD36L1 polypeptide. Structural differences may include, for example, differences in the size, electronegativity, or antigenicity of the mutant CD36L1 polypeptide relative to the normal CD36L1 polypeptide. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook et al, 1989, supra, at Chapter 18. The protein detection and isolation methods employed herein may also be such as those described in Harlow and Lane, for example (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of CD36L1 polypeptides. In situ detection may be accomplished by removing a histological specimen from a subject, and applying thereto a labeled antibody of the present invention. The antibody (or fragment) is preferably applied by overlaying the labeled antibody (or fragment) onto a biological sample. Through the use of such a procedure, it is possible to determine not only the presence of the CD36L1 polypeptide, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labeling an anti-CD36L1 polypeptide specific antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards. Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

If a polymorphic region is located in an exon, either in a coding or non-coding portion of the gene, the identity of the allelic variant can be determined by determining the molecular structure of the mRNA, pre-mRNA, or cDNA. The molecular structure can be determined using any of the above described methods for determining the molecular structure of the genomic DNA.

The methods described herein may be performed, for example, by utilizing pre-packaged diagnostic kits, such as those described above, comprising at least one probe or primer nucleic acid described herein, which may be conveniently used, e.g., to determine whether a subject has or is at risk of developing a disease associated with a specific CD36L1 allelic variant.

Sample nucleic acid to be analyzed by any of the above-described diagnostic and prognostic methods can be obtained from any cell type or tissue of a subject. For example, a subject's bodily fluid (e.g. blood) can be obtained by known techniques (e.g. venipuncture). Alternatively, nucleic acid tests can be performed on dry samples (e.g. hair or skin). Fetal nucleic acid samples can be obtained from maternal blood as described in International Patent Application No. WO91/07660 to Bianchi. Alternatively, amniocytes or chorionic villi may be obtained for performing prenatal testing.

Diagnostic procedures may also be performed in situ directly upon tissue sections (fixed and/or frozen) of subject tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

B. Pharmacogenomics

Knowledge of the identity of the alleles of the CD36L1 gene polymorphic region in a subject (the more CD36L1 genetic profile), alone or in conjunction with information of other genetic defects associated with the same disease (the genetic profile of the particular disease) also allows selection and customization of the therapy, e.g., a particular clinical course of therapy and/or further diagnostic evaluation for a particular disease to the subject's genetic profile. For example, subjects having specific alleles of a CD36L1 gene in combination, may or may not exhibit symptoms of a particular disease or be predisposed to developing symptoms of a particular disease. Further, if those subjects are symptomatic, they may or may not respond to a certain drug, e.g., a specific therapeutic used in the treatment or prevention of abnormal lipid levels, e.g., abnormally high TG level or an abnormally high TG:HDL-C ratio, or a disease or disorder associated with abnormal lipid levels, e.g., a vascular or metabolic disease or disorder. Specific treatments used for the treatment of vascular diseases or disorders, e.g., CAD or MI, include, for example, beta blocker drugs, calcium channel blocker drugs, or nitrate drugs, but may respond to another. Furthermore, they may or may not respond to other treatments, including, for example, use of medical devices for treatment of vascular disease, or surgical and/or non-surgical procedures or courses of treatment. Moreover, if a subject does or does not exhibit symptoms of a particular disease, the subject may or may not benefit from further diagnostic evaluation. Furthermore, knowledge of the identity of the alleles of the CD36L1 gene polymorphic region in a subject, alone or in conjunction with information of other genetic defects associated with abnormal lipid levels of diseases or disorders associated therewith, allows predictions to be made with respect to the response by a subject to a certain therapy, e.g., HRT. For example, if a subject has alleles associated with abnormally high TG and/or high TG:HDL-C ratio, the subject's response to treatment with HRT may be an increased in TG level or TG:HDL-C level or a vascular disease or disorder, e.g., CAD.

Thus, generation of a CD36L1 genetic profile, (e.g., categorization of alterations in a CD36L1 gene which are associated with the development of a particular disease), from a population of subjects, who are symptomatic for a disease or condition that is caused by or contributed to by a defective and/or deficient CD36L1 gene and/or protein (a CD36L1 genetic population profile) and comparison of a subject's CD36L1 profile to the population profile, permits the selection or design of drugs that are expected to be safe and efficacious for a particular subject or subject population (i.e., a group of subjects having the same genetic alteration), as well as the selection or design of a particular clinical course of therapy or further diagnostic evaluations that are expected to be safe and efficacious for a particular subject or subject population.

For example, a CD36L1 population profile can be performed by determining the CD36L1 profile, e.g., the identity of CD36L1 alleles, in a subject population having a disease, which is associated with one or more specific alleles of CD36L1 polymorphic regions. Optionally, the CD36L1 population profile can further include information relating to the response of the population to a CD36L1 therapeutic, using any of a variety of methods, including, monitoring: 1) the severity of symptoms associated with the CD36L1 related disease; 2) CD36L1 gene expression level; 3) CD36L1 mRNA level; and/or 4) CD36L1 protein level, and dividing or categorizing the population based on particular CD36L1 alleles. The CD36L1 genetic population profile can also, optionally, indicate those particular CD36L1 alleles which are present in subjects that are either responsive or non-responsive to a particular therapeutic, clinical course of therapy, or diagnostic evaluation. This information or population profile, is then useful for predicting which individuals should respond to particular drugs, particular clinical courses of therapy, or diagnostic evaluations based on their individual CD36L1 genetic profile.

In a preferred embodiment, the CD36L1 profile is a transcriptional or expression level profile and is comprised of determining the expression level of CD36L1 proteins, alone or in conjunction with the expression level of other genes known to contribute to the same disease at various stages of the disease.

Pharmacogenomic studies can also be performed using transgenic animals. For example, one can produce transgenic mice, e.g., as described herein, which contain a specific allelic variant of a CD36L1 gene. These mice can be created, e.g., by replacing their wild-type CD36L1 gene with an allele of the human CD36L1 gene. The response of these mice to specific CD36L1 particular therapeutics, clinical courses of treatment, and/or diagnostic evaluations can then be determined.

(i) Diagnostic Evaluation

In one embodiment, the polymorphisms of the present invention are used to determine the most appropriate diagnostic evaluation and to determine whether or not a subject will benefit from further diagnostic evaluation. For example, if a subject is heterozygous (CT) at IVS5 and heterozygous (CT) at EX8, or if a subject is homozygous (TT) at IVS5 and heterozygous (CT) at EX8, or the complements thereof, as described herein, that subject is more likely to have or be at risk of developing an abnormally high TG or an abnormally high TG:HDL-C ratio, and therefore to have or to be at a higher than normal risk of developing a vascular disease or disorder such as CAD or MI or a metabolic disease or disorder such as diabetes or obesity. Also for example, if a subject is heterozygous (CT) at IVS5 and heterozygous (CT) at EX8, or if a subject is homozygous (TT) at IVS5 and heterozygous (CT) at EX8, or the complements thereof, as described herein, that subject is more likely to have or be at risk of developing an abnormally high TG or an abnormally high TG:HDL-C ratio, and therefore to have or to be at a higher than normal risk of developing a vascular disease such as CAD or MI if treated with HRT.

Thus, in one embodiment, the invention provides methods for classifying a subject who has, or is at risk for developing, abnormal lipid levels, e.g., abnormally high TG level or an abnormally high TG:HDL-C ratio, or a disease or disorder associated with abnormal lipid levels, e.g., a vascular disease or disorder or a metabolic disease or disorder as a candidate for further diagnostic evaluation for a vascular disease or disorder or a metabolic disease or disorder comprising the steps of determining the CD36L1 genetic profile of the subject, comparing the subject's CD36L1 genetic profile to a CD36L1 genetic population profile, and classifying the subject based on the identified genetic profiles as a subject who is a candidate for further diagnostic evaluation for a vascular disease or disorder or a metabolic disease or disorder. The invention also provides methods for classifying a subject as a candidate for treatment with HRT.

In a preferred embodiment, the subject's CD36L1 genetic profile is determined by identifying the nucleotides present at the EX8 and IVS5 SNPs.

Methods of further diagnostic evaluation include use of vascular imaging devices or procedures such as, for example, angiography, cardiac ultrasound, coronary angiogram, magnetic resonance imagery, nuclear imaging, CT scan, myocardial perfusion imagery, or electrocardiogram, or may include genetic analysis, familial health history analysis, lifestyle analysis, exercise stress tests, or any combination thereof.

ii) Clinical Course of Therapy

In another aspect, the polymorphisms of the present invention are used to determine the most appropriate clinical course of therapy for a subject who has or is at risk of abnormal lipid levels, e.g., abnormally high TG level or an abnormally high TG:HDL-C ratio, or a disease or disorder associated with abnormal lipid levels, e.g., a vascular or metabolic disease or disorder, and will aid in the determination of whether the subject will benefit from such clinical course of therapy, as determined by identification of the polymorphisms of the invention. If a subject is heterozygous (CT) at IVS5 and heterozygous (CT) at EX8, or if a subject is homozygous (TT) at IVS5 and heterozygous (CT) at EX8, or the complements thereof, that subject is more likely to have or be at risk of developing an abnormally high TG or an abnormally high TG:HDL-C ratio, and therefore to have or to be at a higher than normal risk of developing a vascular disease or disorder such as CAD or MI or a metabolic disease or disorder.

Thus, in one aspect, the invention relates to the SNPs identified as described herein, in combination, as well as to the use of these SNPs, and others in these genes, particularly those nearby in linkage disequilibrium with these SNPs, in combination, for prediction of a particular clinical course of therapy for a subject who has, or is at risk for developing, abnormal lipid levels, e.g., abnormally high TG level or an abnormally high TG:HDL-C ratio, or a disease or disorder associated with abnormal lipid levels. In one embodiment, the invention provides a method for determining whether a subject will benefit from a particular course of therapy by determining the presence of the polymorphisms of the invention. For example, the determination of the polymorphisms of the invention, in combination with each other, or in combination with other polymorphisms in the CD36L1 gene or other genes, will aid in the determination of whether an individual will benefit from surgical revascularization and/or will benefit by the implantation of a stent following surgical revascularization, and will aid in the determination of the likelihood of success or failure of a particular clinical course of therapy.

In one embodiment, the invention provides methods for classifying a subject who has, or is at risk for developing, abnormal lipid levels as a candidate for a particular clinical course of therapy for abnormal lipid levels, e.g., abnormally high TG level or an abnormally high TG:HDL-C ratio, or a disease or disorder associated with abnormal lipid levels comprising the steps of determining the CD36L1 genetic profile of the subject; comparing the subject's CD36L1 genetic profile to a CD36L1 genetic population profile; and classifying the subject based on the identified genetic profiles as a subject who is a candidate for a particular clinical course of therapy for abnormal lipid levels.

In another embodiment, the invention provides methods for selecting an effective clinical course of therapy to treat a subject who has, or is at risk for developing, abnormal lipid levels comprising the steps of: determining the CD36L1 genetic profile of the subject; comparing the subject's CD36L1 genetic profile to a CD36L1 genetic population profile; and selecting an appropriate clinical course of therapy for treatment of a subject who has, or is at risk for developing, abnormal lipid levels.

An appropriate clinical course of therapy may include, for example, a lifestyle change, including, for example, a change in diet, exercise, or environment. Other clinical courses of therapy include, but are not limited to, medications, e.g., lipid modulating medication, use of surgical procedures or medical devices. Surgical procedures for the treatment of vascular disorders, includes, for example, surgical revascularization, such as angioplasty, e.g., percutaneous transluminal coronary balloon angioplasty (PTCA), or laser angioplasty, or coronary bypass grafting (CABG). Medical devices used in the treatment or prevention of vascular diseases or disorders, include, for example, devices used in angioplasty, such as balloon angioplasty or laser angioplasty, a device used in coronary revascularization, or a stent, a defibrillator, a pacemaker, or any combination thereof. Medical devices may also be used in combination with modulators of CD36L1 gene expression or CD36L1 protein activity.

C. Monitoring Effects of CD36L1 Therapeutics During Clinical Trials

The present invention provides a method for monitoring the effectiveness of treatment of a subject with a CD36L1 therapeutic e.g., a modulator or agent (e.g, an agonist, antagonist, such as, for example, a peptidomimetic, protein, peptide, nucleic acid, ribozyme, small molecule, or other drug candidate identified, e.g., by the screening assays described herein) comprising the steps of (i) obtaining a preadministration sample from a subject prior to administration of the agent; (ii) detecting the level of expression or activity of a CD36L1 protein, mRNA or gene in the preadministration sample; (iii) obtaining one or more post-administration samples from the subject; (iv) detecting the level of expression or activity of the CD36L1 protein, mRNA or gene in the post-administration samples; (v) comparing the level of expression or activity of the CD36L1 protein, mRNA, or gene in the preadministration sample with those of the CD36L1 protein, mRNA, or gene in the post administration sample or samples; and (vi) altering the administration of the agent to the subject accordingly. For example, increased administration of the agent may be desirable to increase the expression or activity of CD36L1 to higher levels than detected, i.e., to increase the effectiveness of the agent. Alternatively, decreased administration of the agent may be desirable to decrease expression or activity of CD36L1 to lower levels than detected, i.e., to decrease the effectiveness of the agent.

Cells of a subject may also be obtained before and after administration of a CD36L1 therapeutic to detect the level of expression of genes other than CD36L1, to verify that the CD36L1 therapeutic does not increase or decrease the expression of genes which could be deleterious. This can be done, e.g., by using the method of transcriptional profiling. Thus, mRNA from cells exposed in vivo to a CD36L1 therapeutic and mRNA from the same type of cells that were not exposed to the CD36L1 therapeutic could be reverse transcribed and hybridized to a chip containing DNA from numerous genes, to thereby compare the expression of genes in cells treated and not treated with a CD36L1 therapeutic. If, for example a CD36L1 therapeutic turns on the expression of a proto-oncogene in a subject, use of this particular CD36L1 therapeutic may be undesirable.

D. Methods of Treatment

The present invention provides for both prophylactic and therapeutic methods of treating a subject having or likely to develop a disorder associated with specific CD36L1 alleles and/or aberrant CD36L1 expression or activity, e.g., vascular diseases or disorders.

i) Prophylactic Methods

In one aspect, the invention provides a method for preventing a disease or disorder associated with a specific CD36L1 allele such as abnormal lipid levels, e.g., abnormally high TG levels or abnormally high TG:HDL-C levels, and medical conditions resulting therefrom, by administering to the subject an agent which counteracts the unfavorable biological effect of the specific CD36L1 allele. Subjects at risk for such a disease can be identified by a diagnostic or prognostic assay, e.g., as described herein. Administration of a prophylactic agent can occur prior to the manifestation of symptoms associated with specific CD36L1 alleles, such that a disease or disorder is prevented or, alternatively, delayed in its progression. Depending on the identity of the CD36L1 allele in a subject, a compound that counteracts the effect of this allele is administered. The compound can be a compound modulating the activity of CD36L1, e.g., a CD36L1 inhibitor. The treatment can also be a specific lifestyle change, e.g., a change in diet, exercise, or an environmental alteration. In particular, the treatment can be undertaken prophylactically, before any other symptoms are present. Such a prophylactic treatment could thus prevent the development of aberrant lipid levels leading to, for example, a vascular or metabolic disease or disorder. The prophylactic methods are similar to therapeutic methods of the present invention and are further discussed in the following subsections.

(ii) Therapeutic Methods

The invention further provides methods of treating a subject having a disease or disorder associated with a specific allelic variant of a polymorphic region of a CD36L1 gene, e.g., abnormal lipid levels, e.g, abnormally high TG levels and/or abnormally high TG:HDL-C ratios, and diseases or disorders associated therewith, e.g., vascular diseases and disorders, and disorders resulting therefrom (e.g., such as, for example, atherosclerosis, CAD, MI, ischemia, stroke, peripheral vascular diseases, venous thromboembolism and pulmonary embolism, and metabolic diseases or disorders such as, for example, obesity and diabetes).

In one embodiment, the method comprises (a) determining the identity of one or more of the allelic variants of a CD36L1 gene, or preferably, the identity of the nucleotides at the IVS5 SNP and the EX8 SNP; and (b) administering to the subject a compound that compensates for the effect of the specific allelic variant(s). The polymorphic region can be localized at any location of the gene, e.g., in a regulatory element (e.g., in a 5′ upstream regulatory element), in an exon, (e.g., coding region of an exon), in an intron, at an exon/intron border, or in the 3′ UTR. Thus, depending on the site of the polymorphism in the CD36L1 gene, a subject having a specific variant of the polymorphic region which is associated with a specific disease or condition, can be treated with compounds which specifically compensate for the effect of the allelic variant.

In a preferred embodiment, the identity of the nucleotides present at IVS5 and EX8 is determined. If a subject is heterozygous (CT) at IVS5 and heterozygous (CT) at EX8, or if a subject is homozygous (TT) at IVS5 and heterozygous (CT) at EX8, or the complements thereof, that subject is more likely to have or be at risk of developing an abnormally high TG or an abnormally high TG:HDL-C ratio, and therefore to have or to be at a higher than normal risk of developing abnormal lipid levels, e.g., abnormally high TG levels and/or abnormally high TG:HDL-C ratios, and diseases or disorders associated therewith, e.g., vascular diseases and disorders or metabolic disorders.

A mutation can be a substitution, deletion, and/or addition of at least one nucleotide relative to the wild-type allele (i.e., the reference sequence). Depending on where the mutation is located in the CD36L1 gene, the subject can be treated to specifically compensate for the mutation. For example, if the mutation is present in the coding region of the gene and results in a more active CD36L1 protein, the subject can be treated, e.g., by administration to the subject of a modulator, e.g., a therapeutic or course of clinical treatment which treat, prevents, or ameliorates abnormal lipid levels or diseases or disorders associated therewith. Normal CD36L1 protein can also be used to counteract or compensate for the endogenous mutated form of the CD36L1 protein. Normal CD36L1 protein can be directly delivered to the subject or indirectly by gene therapy wherein some cells in the subject are transformed or transfected with an expression construct encoding wild-type CD36L1 protein. Nucleic acids encoding reference human CD36L1 protein are set forth in SEQ ID NO: 1 and SEQ ID NO:3.

Yet in another embodiment, the invention provides methods for treating a subject having a mutated CD36L1 gene, in which the mutation is located in a regulatory region of the gene. Such a regulatory region can be localized in the 5′ upstream regulatory element of the gene, in the 5′ or 3′ untranslated region of an exon, or in an intron. A mutation in a regulatory region can result in increased production of CD36L1 protein, decreased production of CD36L1 protein, or production of CD36L1 having an aberrant tissue distribution. The effect of a mutation in a regulatory region upon the CD36L1 protein can be determined, e.g., by measuring the CD36L1 protein level or mRNA level in cells having a CD36L1 gene having this mutation and which, normally (i.e., in the absence of the mutation) produce CD36L1 protein. The effect of a mutation can also be determined in vitro. For example, if the mutation is in the 5′ upstream regulatory element, a reporter construct can be constructed which comprises the mutated 5′ upstream regulatory element linked to a reporter gene, the construct transfected into cells, and comparison of the level of expression of the reporter gene under the control of the mutated 5′ upstream regulatory element and under the control of a wild-type 5′ upstream regulatory element. Such experiments can also be carried out in mice transgenic for the mutated 5′ upstream regulatory element. If the mutation is located in an intron, the effect of the mutation can be determined, e.g., by producing transgenic animals in which the mutated CD36L1 gene has been introduced and in which the wild-type gene may have been knocked out. Comparison of the level of expression of CD36L1 in the mice transgenic for the mutant human CD36L1 gene with mice transgenic for a wild-type human CD36L1 gene will reveal whether the mutation results in increased, or decreased synthesis of the CD36L1 protein and/or aberrant tissue distribution of CD36L1 protein. Such analysis could also be performed in cultured cells, in which the human mutant CD36L1 gene is introduced and, e.g., replaces the endogenous wild-type CD36L1 gene in the cell. Thus, depending on the effect of the mutation in a regulatory region of a CD36L1 gene, a specific treatment can be administered to a subject having such a mutation. Accordingly, if the mutation results in increased CD36L1 protein levels, the subject can be treated by administration of a compound which reduces CD36L1 protein production, e.g., by reducing CD36L1 gene expression or a compound which inhibits or reduces the activity of CD36L1.

A correlation between drug responses and specific alleles of CD36L1 can be shown, for example, by clinical studies wherein the response to specific drugs of subjects having different allelic variants of a polymorphic region of a CD36L1 gene is compared. Such studies can also be performed using animal models, such as mice having various alleles of a human CD36L1 gene and in which, e.g., the endogenous CD36L1 gene has been inactivated such as by a knock-out mutation. Test drugs are then administered to the mice having different human CD36L1 alleles and the response of the different mice to a specific compound is compared. Accordingly, the invention provides assays for identifying the drug which will be best suited for treating a specific disease or condition in a subject. For example, it will be possible to select drugs which will be devoid of toxicity, or have the lowest level of toxicity possible for treating a subject having a disease or condition.

Other Uses For the Nucleic Acid Molecules of the Invention

The identification of different alleles of CD36L1 can also be useful for identifying an individual among other individuals from the same species. For example, DNA sequences can be used as a fingerprint for detection of different individuals within the same species (Thompson, J. S. and Thompson, eds., Genetics in Medicine, WB Saunders Co., Philadelphia, Pa. (1991)). This is useful, for example, in forensic studies and paternity testing, as described below.

A. Forensics

Determination of which specific allele occupies a set of one or more polymorphic sites in an individual identifies a set of polymorphic forms that distinguish the individual from others in the population. See generally National Research Council, The Evaluation of Forensic DNA Evidence (Eds. Pollard et al., National Academy Press, DC, 1996). The more polymorphic sites that are analyzed, the lower the probability that the set of polymorphic forms in one individual is the same as that in an unrelated individual. Preferably, if multiple sites are analyzed, the sites are unlinked. Thus, the polymorphisms of the invention can be used in conjunction with known polymorphisms in distal genes. Preferred polymorphisms for use in forensics are biallelic because the population frequencies of two polymorphic forms can usually be determined with greater accuracy than those of multiple polymorphic forms at multi-allelic loci.

The capacity to identify a distinguishing or unique set of polymorphic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers is the same in the sample as in the suspect, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

p(ID) is the probability that two random individuals have the same polymorphic or allelic form at a given polymorphic site. For example, in biallelic loci, four genotypes are possible: AA, AB, BA, and BB. If alleles A and B occur in a haploid genome of the organism with frequencies x and y, the probability of each genotype in a diploid organism is (see WO 95/12607):

Homozygote: p(AA)=x²

Homozygote: p(BB)=y²=(1−x)²

Single Heterozygote: p(AB)=p(BA)=xy=x(1−x)

Both Heterozygotes: p(AB+BA)=2xy=2x(1−x)

The probability of identity at one locus (i.e., the probability that two individuals, picked at random from a population will have identical polymorphic forms at a given locus) is given by the equation: p(ID)=(x²).

These calculations can be extended for any number of polymorphic forms at a given locus. For example, the probability of identity p(ID) for a 3-allele system where the alleles have the frequencies in the population of x, y, and z, respectively, is equal to the sum of the squares of the genotype frequencies: P(ID)=x⁴+(2xy)²+(2yz)²+(2xz)²+z⁴+y⁴.

In a locus of n alleles, the appropriate binomial expansion is used to calculate p(ID) and p(exc).

The cumulative probability of identity (cum p(ID)) for each of multiple unlinked loci is determined by multiplying the probabilities provided by each locus:

cum p(ID)=p(ID1)p(ID2)p(ID3) . . . p(IDn).

The cumulative probability of non-identity for n loci (i.e., the probability that two random individuals will be difference at 1 or more loci) is given by the equation:

cum p(nonID)=1−cum p(ID).

If several polymorphic loci are tested, the cumulative probability of non-identity for random individuals becomes very high (e.g., one billion to one). Such probabilities can be taken into account together with other evidence in determining the guilt or innocence of the suspect.

B. Paternity Testing

The object of paternity testing is usually to determine whether a male is the father of a child. In most cases, the mother of the child is known, and thus, it is possible to trace the mother's contribution to the child's genotype. Paternity testing investigates whether the part of the child's genotype not attributable to the mother is consistent to that of the putative father. Paternity testing can be performed by analyzing sets of polymorphisms in the putative father and in the child.

If the set of polymorphisms in the child attributable to the father does not match the set of polymorphisms of the putative father, it can be concluded, barring experimental error, that that putative father is not the real father. If the set of polymorphisms in the child attributable to the father does match the set of polymorphisms of the putative father, a statistical calculation can be performed to determine the probability of a coincidental match.

The probability of parentage exclusion (representing the probability that a random male will have a polymorphic form at a given polymorphic site that makes him incompatible as the father) is given by the equation (see WO 95/12607): p(exc)=xy(1−xy), where x and y are the population frequencies of alleles A and B of a biallelic polymorphic site.

(At a trial lelic site p(exc)=xy(1−-xy)+yz(1−yz)+xz(1−xz)+3xyz(1−xyz)), where x, y, and z and the respective populations frequencies of alleles A, B, and C).

The probability of non-exclusion is: p(non-exc)=1−-p(exc).

The cumulative probability of non-exclusion (representing the values obtained when n loci are is used) is thus:

Cum p(non-exc)=p(non-exc1)p(non-exc2)p(non-exc3) . . . p(non-excn).

The cumulative probability of the exclusion for n loci (representing the probability that a random male will be excluded: cum p(exc)=1−cum p(non-exc).

If several polymorphic loci are included in the analysis, the cumulative probability of exclusion of a random male is very high. This probability can be taken into account in assessing the liability of a putative father whose polymorphic marker set matches the child's polymorphic marker set attributable to his or her father.

C. Kits

As set forth herein, the invention provides methods, e.g., diagnostic and therapeutic methods, e.g., for determining the type of allelic variant of a polymorphic region present in a CD36L1 gene, such as a human CD36L1 gene. In preferred embodiments, the methods use probes or primers comprising nucleotide sequences which are complementary to a polymorphic region of a CD36L1 gene. In a preferred embodiment, the methods use probes or primers comprising nucleotide sequences which are complementary to a polymorphic region of a CD36L1 gene. Accordingly, the invention provides kits for performing these methods. In a preferred embodiment, the kit comprises probes or primers comprising nucleotide sequences which are complementary to one or more of the variant alleles at nucleotide position 41 of exon 8 or 54 of intron 5 of the CD36L1 gene, or the complements thereof. For example, if a subject is heterozygous (CT) at IVS5 and heterozygous (CT) at EX8, or if a subject is homozygous (TT) at IVS5 and heterozygous (CT) at EX8, or the complements thereof, that subject is more likely to have or be at risk of developing an abnormally high TG or an abnormally high TG:HDL-C ratio, and therefore to have or to be at a higher than normal risk of developing a vascular disease or disorder such as CAD or MI or a metabolic disease or disorder such as diabetes or obesity.

In a preferred embodiment, the invention provides a kit for determining whether a subject has or is at risk of developing a disease or condition associated with a specific allelic variant of a CD36L1 polymorphic region. In an even more preferred embodiment, the disease or disorder is characterized by an abnormal CD36L1 activity. In an even more preferred embodiment, the invention provides a kit for determining whether a subject has or is or is not at risk of developing a abnormal lipid levels, e.g, abnormally high TG levels and/or abnormally high TG:HDL-C ratios or a disease or disorder associated therewith, e.g., a vascular disease or disorder, e.g., atherosclerosis, CAD, MI, ischemia, stroke, peripheral vascular diseases, venous thromboembolism and pulmonary embolism or a metabolic disease or disorder.

A preferred kit provides reagents for determining whether a subject is likely to develop abnormal lipid levels, e.g., abnormally high TG levels and/or abnormally high TG:HDL-C ratios, and diseases or disorders associated therewith, e.g., vascular diseases and disorders.

Preferred kits comprise at least one probe or primer which is capable of specifically hybridizing under stringent conditions to a CD36L1 sequence or polymorphic region and instructions for use. The kits preferably comprise at least one of the above described nucleic acids. Preferred kits for amplifying at least a portion of a CD36L1 gene comprise at least two primers, at least one of which is capable of hybridizing to an allelic variant sequence.

The kits of the invention can also comprise one or more control nucleic acids or reference nucleic acids, such as nucleic acids comprising a CD36L1 intronic sequence. For example, a kit can comprise primers for amplifying a polymorphic region of a CD36L1 gene and a control DNA corresponding to such an amplified DNA and having the nucleotide sequence of a specific allelic variant. Thus, direct comparison can be performed between the DNA amplified from a subject and the DNA having the nucleotide sequence of a specific allelic variant. In one embodiment, the control nucleic acid comprises at least a portion of a CD36L1 gene of an individual who does not have abnormal lipid levels, e.g., abnormally high TG levels and/or abnormally high TG:HDL-C ratios, or a disease or disorder associated with an aberrant CD36L1 activity.

Yet other kits of the invention comprise at least one reagent necessary to perform the assay. For example, the kit can comprise an enzyme. Alternatively the kit can comprise a buffer or any other necessary reagent.

D. Electronic Apparatus Readable Media and Arrays

Electronic apparatus readable media comprising polymorphisms of the present invention is also provided. As used herein, “electronic apparatus readable media” and “computer readable media,” which are used interchangeably herein, refer to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon a marker of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the polymorphisms of the present invention.

A variety of software programs and formats can be used to store the polymorphisms information of the present invention on the electronic apparatus readable medium. For example, the polymorphic sequence can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of data processor structuring formats (e.g., text file or database) may be employed in order to obtain or create a medium having recorded thereon the markers of the present invention.

By providing the polymorphisms of the invention in readable form, in combination, one can routinely access the polymorphism information for a variety of purposes. For example, one skilled in the art can use the sequences of the polymorphisms of the present invention in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has abnormal lipid levels, e.g., abnormally high TG levels and/or abnormally high TG:HDL-C ratios or a pre-disposition to abnormal lipid levels, e.g., abnormally high TG levels and/or abnormally high TG:HDL-C ratios, wherein the method comprises the steps of determining the presence or absence of a polymorphism and based on the presence or absence of the polymorphism, determining whether the subject has abnormal lipid levels, e.g., abnormally high TG levels and/or abnormally high TG:HDL-C ratios or a predisposition to abnormal lipid levels, e.g., abnormally high TG levels and/or abnormally high TG:HDL-C ratios and/or recommending a particular clinical course of therapy or diagnostic evaluation for the abnormal lipid levels, e.g., abnormally high TG levels and/or abnormally high TG:HDL-C ratios or pre-disease condition.

The present invention further provides in an electronic system and/or in a network, a method for determining whether a subject has a vascular disease or a pre-disposition to vascular disease associated with a polymorphism as described herein wherein the method comprises the steps of determining the presence or absence of the polymorphism, and based on the presence or absence of the polymorphism, determining whether the subject has abnormal lipid levels, e.g., abnormally high TG levels and/or abnormally high TG:HDL-C ratios or a pre-disposition to a abnormal lipid levels, e.g., abnormally high TG levels and/or abnormally high TG:HDL-C ratios, and/or recommending a particular treatment for the abnormal lipid levels, e.g., abnormally high TG levels and/or abnormally high TG:HDL-C ratios or pre-disease condition. The method may further comprise the step of receiving phenotypic information associated with the subject and/or acquiring from a network phenotypic information associated with the subject.

The present invention also provides in a network, a method for determining whether a subject has vascular disease or a pre-disposition to abnormal lipid levels, e.g., abnormally high TG levels and/or abnormally high TG:HDL-C ratios associated with a polymorphism, said method comprising the steps of receiving information associated with the polymorphism, receiving phenotypic information associated with the subject, acquiring information from the network corresponding to the polymorphism and/or disease, and based on one or more of the phenotypic information, the polymorphism, and the acquired information, determining whether the subject has a abnormal lipid levels, e.g., abnormally high TG levels and/or abnormally high TG:HDL-C ratios or a pre-disposition to abnormal lipid levels, e.g., abnormally high TG levels and/or abnormally high TG:HDL-C ratios. The method may further comprise the step of recommending a particular treatment for the abnormal lipid levels, e.g., abnormally high TG levels and/or abnormally high TG:HDL-C ratios or pre-disease condition.

The present invention also provides a method for determining whether a subject has a vascular disease or a pre-disposition to a abnormal lipid levels, e.g., abnormally high TG levels and/or abnormally high TG:HDL-C ratios, said method comprising the steps of receiving information associated with the polymorphism, receiving phenotypic information associated with the subject, acquiring information from the network corresponding to the polymorphism and/or vascular disease, and based on one or more of the phenotypic information, the polymorphism, and the acquired information, determining whether the subject has abnormal lipid levels, e.g., abnormally high TG levels and/or abnormally high TG:HDL-C ratios disease or a pre-disposition to abnormal lipid levels, e.g., abnormally high TG levels and/or abnormally high TG:HDL-C ratios. The method may further comprise the step of recommending a particular treatment for abnormal lipid levels, e.g, abnormally high TG levels and/or abnormally high TG:HDL-C ratios or pre-disease condition.

E. Personalized Health Assessment

Methods and systems of assessing personal health and risk for disease, e.g., abnormal lipid levels, e.g., abnormally high TG levels and/or abnormally high TG:HDL-C ratios, in a subject, using the polymorphisms and association of the instant invention are also provided. The methods provide personalized health care knowledge to individuals as well as to their health care providers, as well as to health care companies. It will be appreciated that the term “health care providers” is not limited to physicians but can be any source of health care. The methods and systems provide personalized information including a personal health assessment report that can include a personalized molecular profile, e.g., a CD36L1 genetic profile, a health profile, or both. Overall, the methods and systems as described herein provide personalized information for individuals and patient management tools for healthcare providers and/or subjects using a variety of communications networks such as, for example, the Internet. U.S. Patent Application Serial No. 60/266,082, filed Feb. 1, 2001, entitled “Methods and Systems for Personalized Health Assessment,” further describes personalized health assessment methods, systems, and apparatus, and is expressly incorporated herein by reference.

In one aspect, the invention provides an Internet-based method for assessing a subject's risk for abnormal lipid levels, e.g., abnormally high TG levels and/or abnormally high TG:HDL-C ratios. In one embodiment, the method comprises obtaining a biological sample from a subject, analyzing the biological sample to determine the presence or absence of a polymorphic region of CD36L1, and providing results of the analysis to the subject via the Internet, wherein the presence of a polymorphic region of CD36L1 indicates an increased or decreased risk for abnormal lipid levels, e.g., abnormally high TG levels and/or abnormally high TG:HDL-C ratios. In another embodiment, the method comprises analyzing data from a biological sample from a subject relating to the presence or absence of a polymorphic region of CD36L1 and providing results of the analysis to the subject via the Internet, wherein the presence of a polymorphic region of CD36L1 indicates an increased or decreased risk for abnormal lipid levels, e.g., abnormally high TG levels and/or abnormally high TG:HDL-C ratios.

It will be appreciated that the phrase “wherein the presence of a polymorphic region of CD36L1 indicates an increased risk for abnormal lipid levels, e.g., abnormally high TG levels and/or abnormally high TG:HDL-C ratios” includes an increased or higher than normal risk of developing abnormal lipid levels, e.g., abnormally high TG levels and/or abnormally high TG:HDL-C ratios indicated by a subject being heterozygous (CT) at IVS5 and heterozygous (CT) at EX8, or the complements thereof, or a subject being homozygous (TT) at IVS5 and heterozygous (CT) at EX8, or the complements thereof, or the complements thereof, wherein the presence of the combination of alleles at EX8 and IVS5 indicates an association with abnormally high TG or an abnormally high TG:HDL-C ratio.

The terms “Internet” and/or “communications network” as used herein refer to any suitable communication link, which permits electronic communications. It should be understood that these terms are not limited to “the Internet” or any other particular system or type of communication link. That is, the terms “Internet” and/or “communications network” refer to any suitable communication system, including extra-computer system and intra-computer system communications. Examples of such communication systems include internal busses, local area networks, wide area networks, point-to-point shared and dedicated communications, infra-red links, microwave links, telephone links, CATV links, satellite and radio links, and fiber-optic links. The terms “Internet” and/or “communications network” can also refer to any suitable communications system for sending messages between remote locations, directly or via a third party communication provider such as AT&T. In this instance, messages can be communicated via telephone or facsimile or computer synthesized voice telephone messages with or without voice or tone recognition, or any other suitable communications technique.

In another aspect, the methods of the invention also provide methods of assessing a subject's risk for abnormal lipid levels, e.g., abnormally high TG levels and/or abnormally high TG:HDL-C ratios. In one embodiment, the method comprises obtaining information from the subject regarding the polymorphic region of a CD36L1 gene, through e.g., obtaining a biological sample from the individual, analyzing the sample to obtain the subject's CD36L1 genetic profile, representing the CD36L1 genetic profile information as digital genetic profile data, electronically processing the CD36L1 digital genetic profile data to generate a risk assessment report for abnormal lipid levels, e.g., abnormally high TG levels and/or abnormally high TG:HDL-C ratios, and displaying the risk assessment report on an output device, where,the presence of a polymorphic region of CD36L1 indicates an increased risk for abnormal lipid levels, e.g., abnormally high TG levels and/or abnormally high TG:HDL-C ratios. In another embodiment, the method comprises analyzing a subject's CD36L1 genetic profile, representing the CD36L1 genetic profile information as digital genetic profile data, electronically processing the CD36L1 digital genetic profile data to generate a risk assessment report for abnormal lipid levels, e.g., abnormally high TG levels and/or abnormally high TG:HDL-C ratios, and displaying the risk assessment report on an output device, where the presence of a polymorphic region of CD36L1 indicates an increased risk for abnormal lipid levels, e.g., abnormally high TG levels and/or abnormally high TG:HDL-C ratios. Additional health information may be provided and can be utilized to generate the risk assessment report. Such information includes, but is not limited to, information regarding one or more of age, sex, ethnic origin, diet, sibling health, parental health, clinical symptoms, personal health history, blood test data, weight, and alcohol use, drug use, nicotine use, and blood pressure.

The CD36L1 digital genetic profile data may be transmitted via a communications network, e.g., the Internet, to a medical information system for processing.

In yet another aspect the invention provides a medical information system for assessing a subject's risk for abnormal lipid levels, e.g., abnormally high TG levels and/or abnormally high TG:HDL-C ratios comprising a means for obtaining information from the subject regarding the polymorphic region of a CD36L1 gene, through e.g., obtaining a biological sample from the individual to obtain a CD36L1 genetic profile, a means for representing the CD36L1 genetic profile as digital molecular data, a means for electronically processing the CD36L1 digital genetic profile to generate a risk assessment report for abnormal lipid levels, e.g., abnormally high TG levels and/or abnormally high TG:HDL-C ratios, and a means for displaying the risk assessment report on an output device, where the presence of a polymorphic region of CD36L1 indicates an increased risk for abnormal lipid levels, e.g., abnormally high TG levels and/or abnormally high TG:HDL-C ratios.

In another aspect, the invention provides a computerized method of providing medical advice to a subject comprising obtaining information from the subject regarding the polymorphic region of a CD36L1 gene, through e.g., obtaining a biological sample from the subject, analyzing the subject's biological sample to determine the subject's CD36L1 genetic profile, and, based on the subject's CD36L1 genetic profile, determining the subject's risk for abnormal lipid levels, e.g., abnormally high TG levels and/or abnormally high TG:HDL-C ratios. Medical advice may be then provided electronically to the subject, based on the subject's risk for abnormal lipid levels, e.g., abnormally high TG levels and/or abnormally high TG:HDL-C ratios. The medical advice may comprise, for example, recommending one or more of the group consisting of: further diagnostic evaluation, use of medical or surgical devices, administration of medication, e.g lipid modulating medications, or lifestyle change, e.g., diet or exercise change. Additional health information may also be obtained from the subject and may also be used to provide the medical advice.

In another aspect, the invention includes a method for self-assessing risk for a abnormal lipid levels, e.g., abnormally high TG levels and/or abnormally high TG:HDL-C ratios. The method comprises providing information from the subject regarding the polymorphic region of a CD36L1 gene, through e.g., providing a biological sample for genetic analysis, and accessing an electronic output device displaying results of the genetic analysis, thereby self-assessing risk for abnormal lipid levels, e.g., abnormally high TG levels and/or abnormally high TG:HDL-C ratios, where the presence of a polymorphic region of CD36L1 indicates an increased risk for abnormal lipid levels, e.g., abnormally high TG levels and/or abnormally high TG:HDL-C ratios.

In another aspect, the invention provides a method of self-assessing risk for abnormal lipid levels, e.g., abnormally high TG levels and/or abnormally high TG:HDL-C ratios comprising providing information from the subject regarding the polymorphic region of a CD36L1 gene, through e.g., providing a biological sample, accessing CD36L1 digital genetic profile data obtained from the biological sample, the CD36L1 digital genetic profile data being displayed via an output device, where the presence of a polymorphic region of CD36L1 indicates an increased risk for abnormal lipid levels, e.g., abnormally high TG levels and/or abnormally high TG:HDL-C ratios.

An output device may be, for example, a CRT, printer, or website. An electronic output device may be accessed via the Internet.

The biological sample may be obtained from the individual at a laboratory company. In one embodiment, the laboratory company processes the biological sample to obtain CD36L1 genetic profile data, represents at least some of the CD36L1 genetic profile data as digital genetic profile data, and transmits the CD36L1 digital genetic profile data via a communications network to a medical information system for processing. The biological sample may also be obtained from the subject at a draw station. A draw station processes the biological sample to obtain CD36L1 genetic profile data and transfers the data to a laboratory company. The laboratory company then represents at least some of the CD36L1 genetic profile data as digital genetic profile data, and transmits the CD36L1 digital genetic profile data via a communications network to a medical information system for processing.

In another aspect, the invention provides a method for a health care provider to generate a personal health assessment report for an individual. The method comprises counseling the individual to provide a biological sample and authorizing a draw station to take a biological sample from the individual and transmit molecular information from the sample to a laboratory company, where the molecular information comprises the presence or absence of a polymorphic region of CD36L1. The health care provider then requests the laboratory company to provide digital molecular data corresponding to the molecular information to a medical information system to electronically process the digital molecular data and digital health data obtained from the individual to generate a health assessment report, receives the health assessment report from the medical information system, and provides the health assessment report to the individual.

In still another aspect, the invention provides a method of assessing the health of an individual. The method comprises obtaining health information from the individual using an input device (e.g., a keyboard, touch screen, hand-held device, telephone, wireless input device, or interactive page on a website), representing at least some of the health information as digital health data, obtaining a biological sample from the individual, and processing the biological sample to obtain molecular information, where the molecular information comprises the presence or absence of a polymorphic region of CD36L1. At least some of the molecular information and health data is then presented as digital molecular data and electronically processed to generate a health assessment report. The health assessment report is then displayed on an output device. The health assessment report can comprise a digital health profile of the individual. The molecular data can comprise protein sequence data, and the molecular profile can comprise a proteomic profile. The molecular data can also comprise information regarding one or more of the absence, presence, or level, of one or more specific proteins, polypeptides, chemicals, cells, organisms, or compounds in the individual's biological sample. The molecular data may also comprise, e.g., nucleic acid sequence data, and the molecular profile may comprise, e.g., a genetic profile.

In yet another embodiment, the method of assessing the health of an individual further comprises obtaining a second biological sample or a second health information at a time after obtaining the initial biological sample or initial health information, processing the second biological sample to obtain second molecular information, processing the second health information, representing at least some of the second molecular information as digital second molecular data and second health information as digital health information, and processing the molecular data and second molecular data and health information and second health information to generate a health assessment report. In one embodiment, the health assessment report provides information about the individual's predisposition for abnormal lipid levels, e.g., abnormally high TG levels and/or abnormally high TG:HDL-C ratios, and options for risk reduction.

Options for risk reduction comprise, for example, one or more of diet, exercise, one or more vitamins, one or more drugs, cessation of nicotine use, and cessation of alcohol use. wherein the health assessment report provides information about treatment options for a particular disorder. Treatment options comprise, for example, one or more of diet, one or more drugs, physical therapy, and surgery. In one embodiment, the health assessment report provides information about the efficacy of a particular treatment regimen and options for therapy adjustment.

In another embodiment, electronically processing the digital molecular data and digital health data to generate a health assessment report comprises using the digital molecular data and/or digital health data as inputs for an algorithm or a rule-based system that determines whether the individual is at risk for a specific disorder, e.g., abnormal lipid levels, e.g, abnormally high TG levels and/or abnormally high TG:HDL-C ratios. Electronically processing the digital molecular data and digital health data may also comprise using the digital molecular data and digital health data as inputs for an algorithm or a rule-based system based on one or more databases comprising stored digital molecular data and/or digital health data relating to one or more disorders, e.g., abnormal lipid levels, e.g., abnormally high TG levels and/or abnormally high TG:HDL-C ratios.

In another embodiment, processing the digital molecular data and digital health data comprises using the digital molecular data and digital health data as inputs for an algorithm or a rule-based system based on one or more databases comprising: (i) stored digital molecular data and/or digital health data from a plurality of healthy individuals, and (ii) stored digital molecular data and/or digital health data from one or more pluralities of unhealthy individuals, each plurality of individuals having a specific disorder. At least one of the databases can be a public database. In one embodiment, the digital health data and digital molecular data are transmitted via, e.g., a communications network, e.g., the Internet, to a medical information system for processing.

A database of stored molecular data and health data, e.g., stored digital molecular data and/or digital health data, from a plurality of individuals, is further provided. A database of stored digital molecular data and/or digital health data from a plurality of healthy individuals, and stored digital molecular data and/or digital health data from one or more pluralities of unhealthy individuals, each plurality of individuals having a specific disorder, e.g., abnormal lipid levels, e.g., abnormally high TG levels and/or abnormally high TG:HDL-C ratios, is also provided.

The new methods and systems of the invention provide healthcare providers with access to ever-growing relational databases that include both molecular data and health data that is linked to specific disorders, e.g., abnormal lipid levels, e.g, abnormally high TG levels and/or abnormally high TG:HDL-C ratios. In addition public medical knowledge is screened and abstracted to provide concise, accurate information that is added to the database on an ongoing basis. In addition, new relationships between particular SNPs, e.g., SNPs associated with abnormal lipid levels, e.g., abnormally high TG levels and/or abnormally high TG:HDL-C ratios, or genetic mutations and specific discords are added as they are discovered.

The present invention is further illustrated by the following examples which should not be construed as limiting in any way. The contents of all cited references (including, without limitation, literature references, issued patents, published patent applications and database records including Genbank™ records) as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

### Association of Common Polymorphisms at the CD36L1 Gene with Abnormal Lipids in Women with Familial, Premature Coronary Artery Disease

METHODS

Study Populations

Subjects were drawn from the GeneQuest study, a collection of nuclear families consisting of affected sibling pairs and living parents ascertained for premature CAD at fifteen medical centers in the United States. Each proband was required to have developed CAD by age 45 if male, or age 50 if female, as manifest by either a myocardial infarction, surgical or percutaneous coronary revascularization, or a coronary angiogram with evidence of at least a 70% stenosis in a major epicardial artery. At least one living sibling also had to fulfill these qualifying criteria for the family to be enrolled. Probands were identified through patient registries as well as new admissions. The protocol was approved by the institutional review board at each participating institution. All patients gave informed consent to participate.

Patients meeting eligibility criteria were invited to the hospital clinic for physical evaluation and blood draw. Clinical data, including anthropometric measures such as body mass index (BMI; kg/m²), were gathered for each subject on the day of enrollment. Cardiac events and patient medical histories for all affected individuals were confirmed through medical records. Non-fasting blood samples were obtained by drawing into tubes containing EDTA. Plasma HDL-C, total cholesterol and TG levels were measured using standard procedures at a central laboratory at the Cleveland Clinic. Genomic DNA was isolated from peripheral blood lymphocytes using the Puregene™ kit (Gentra Systems, Inc.™) according to manufacturer's suggested protocol at a commercial laboratory.

For the purpose of the current study, a case series of unrelated individuals with CAD was selected such that only one from each family was represented, giving preference to the sibling with the earlier age of onset. The case series was limited to Caucasian individuals as they represented the majority of the collection.

Genotyping CD36L1 Variants

Six previously identified SNPs in CD36L1 were evaluated: a missense (G→A) at nucleotide position 146 in exon 1 resulting in a change from glycine to serine amino acid 2 in exon 1 (EX1; alleles EX1G and EX1A), a missense (G→A) at nucleotide position 119 in exon 3 resulting in a change from valine to isoleucine at amino acid 135 in exon 3 (EX3; alleles EX3G and EX3A), an intronic SNP (C→T) at nucleotide position 54 of intron 5 (IVS5; alleles IVS5C and IVS5T), a silent mutation (C→T) at amino acid 301 in exon 7 (EX7; alleles EX7C and EX7T), a silent mutation (C→T) at nucleotide position 41, amino acid 350 in exon 8 (EX8; alleles EX8C and EX8T), and an intronic SNP (C→G) 41bp upstream from the start of exon 11 (IVS10; alleles IVS10C and IVS10G). Genotypes were obtained using the 5′ nuclease assay with allele specific TaqMan probes (Livak, et al. (1999) Genet Anal 14(5-6 :143-9).

Statistical Analysis

Linkage disequilibrium was assessed with the normalized disequilibrium parameter (Lewontin, R. C. (1964) Genetics 49:49-67). D′, using the EM algorithm (Excoffer L and Slatkin, M. (1995) Mol Biol Evol. 12:921-927). All other analyses were performed using the SAS statistical package version 6.12 (SAS Institute Inc.). The association of CD36L1 genotypes with HDL-C, TG and the TG:HDL-C ratio was assessed using the general linear model procedure (PROC GLM). Since TG levels are not normally distributed, both TG and the ratio of TG:HDL-C were log-tranformed prior to analysis. Each SNP was defined as a ‘class’ variable, which allowed the effect of each of the 3 possible genotypes (homozygous variant, heterozygous and homozygous wildtype) to be evaluated separately. Because the homozygous variant genotype for IVS10 was found in only one person, this genotype was grouped with heterozygotes for analysis. Hardy-Weinberg equilibrium was assessed with a chi square goodness of fit.

Combinations of SNPs were evaluated by use of an interaction term as an indirect means of assessing the effect of haplotypes. Only those SNP pairs showing significant linkage disequilibrium (D′>0.30 and p<0.05) were evaluated in this way. The effect of interaction between sex and genotype combinations on lipid levels was tested with 3-way interaction terms which included sex and a single pair of polymorphisms, as well as all possible two-way interactions and main effects of these interactions. Some 3-way and all higher order interactions were not evaluated due to small sample size. When significant interaction was found with sex, further analyses were carried out for men and women separately.

Results

The study population included 113 female and 258 male CAD patients. At enrollment, subjects ranged in age from 29 to 72 years with a mean of 47.3 years for women and 47.7 years for men. Patients were retrospectively ascertained with an average time from their qualifying event to enrollment of 6.8 years (range 0-30 years) for women and 9.3 years (range 0-42 years) for men. Because all subjects were chosen from families originally ascertained for CAD, they are enriched for CAD risk factors including low HDL-C, high TG, high BMI and type 2 diabetes mellitus (NIDDM). The mean BMI for women was 29.1 (range 16-53) and for men 29.7 (range 19-61). Fifteen percent of women and 8% of men had NIDDM. The distribution of HDL-C and TG levels in the population are shown in FIG. 1 for men and FIG. 2 for women. Forty-seven percent of women and 63% of men had low HDL-C (<40 mg/dl). Among those with low HDL-C, 57% of women and 66% of men also had elevated TG levels (>200 mg/dl).

Variant allele frequencies were 0.12 for EX1A, 0.006 for EX3A, 0.08 for IVS5T, 0.01 for EX7T, 0.51 for EX8C, 0.06 for IVSIOG. All SNPs were in Hardy-Weinberg equilibrium (p>0.10). Because EX3A and EX7T were rare, they were not included in statistical analyses. No evidence was found for linkage disequilibrium (a non-random association or correlation between SNPs) between the EX1 polymorphism and any of the other three polymorphisms (D′=0.21, 0.23 and 0.36 for linkage disequilibrium with IVS5, EX8 and IVS10, respectively). Strong, significant linkage disequilibrium was found between three pairs: EX8 and IVS5 (D′=0.68, p<0.0001), EX8 and IVS10 (D′=0.65, p=0.006), and IVS5 and IVS10 (D′=0.99, p=0.05), suggesting that these three variants may be inherited together as a haplotype. The combined effects of these SNP pairs were evaluated further.

Results of linear regression analysis revealed that HDL-C levels were associated with combinations of genotypes at two sites in CD36L1 and that the effect was modified by sex. Significant interaction was found for sex, the IVS5 SNP and the EX8 SNP (sex*IVS5*EX8, p=0.03). Similar analyses examining TG as the dependent variable were carried out and the interaction term sex*IVS5*EX8 was found to be significant again (p=0.006). Finally, to account for the combined effect of low HDL-C and high TG levels, the ratio of TG:HDL-C was examined and once more significant interaction for sex*IVS5*EX8 (p=0.003) was found. Controlling for NIDDM status and BMI in these models did not alter the significance substantially.

General linear models run for men and women separately revealed striking differences. Significant interaction between the IVS5 and EX8 SNPs (IVS5*EX8) was found for HDL-C (p=0.01), TG (p=0.0004) and for the TG:HDL-C ratio (p<0.0001) in women. In men, no significant interactions in the association of HDL-C (p=0.81), TG (p=0.60) or TG:HDL-C (p=0.61) were found for this or any other combination of SNPs.

Mean levels of HDL-C, TG and the TG:HDL-C ratio in women by combined IVS5 and EX8 genotypes are presented in Table I. Significant differences were found between genotype combinations of IVS5 and EX8 for TG (p=0.001) and TG:HDL-C ratio (p=0.0003). The association with HDL-C was borderline (p=0.07). The amount of variance in serum lipid levels explained by the combined genotypes of EX8 and IVS5 in women was 19.4% for TG, 21.9% for the ratio of TG:HDL-C and 10.1% for HDL-C. The two most infrequent combinations of genotypes gave the lowest mean level of HDL-C, highest mean level of TG and the highest TG:HDL-C ratio. The one woman who was heterozygous at IVS5 (CT genotype) and homozygous (TT) at EX8 had an HDL-C of 36 mg/dl, TG of 797 mg/dl and TG:HDL-C ratio of 22:1. Three women heterozygous at both IVS5 (CT) and EX8 (CT) had the lowest mean level of HDL-C (25.0 mg/dl), high mean level of TG (778 mg/dl) and highest TG:HDL-C ratio (38:1). Other characteristics of these four women are shown in Table IV, below.

TABLE IV
Additional characteristics of the four women with IVS5-EX8
combinations associated with extreme TG:HDL-C ratios.

| | Patient A | Patient B | Patient C | Patient D |
|---|---|---|---|---|
| IVS5 genotype | CT | CT | CT | CT |
| EX8 genotype | TT | CT | CT | CT |
| HDL-C (mg/dl) 36 | 37 | 18 | 20 | |
| TG (mg/dl) | 797 | 238 | 722 | 1374 |
| BMI | 33 | 29 | 33 | 34 |
| Diabetes | NIDDM | No | NIDDM | IDDM |
| Age at enrollment | 44 | 45 | 52 | 49 |
| Qualifying event | MI | MI | MI | MI |
| Age at event | 32 | 45 | 34 | 39 |
| Hypertension | Yes | No | No | Yes |

Because the serum lipid levels of the three women heterozygous at both IVS5 and EX8 were extreme, it was hypothesized that women with this combination (3% in the population studied) may experience reduced fitness and therefore be subject to negative selection. To test this indirectly, the frequency of the combination in men and women from GeneQuest were compared and it was found that there was a significantly higher frequency in men (9%, p=0.009). The frequency between men and women >50 years old from a general U.S. Caucasian population sample was also compared and again it a significantly lower frequency in women (4%) compared to men (15%, p=0.03) was found.

To determine if the observed associations could have been detected by examining anything less than a 3-way interaction, the association of each of the four individual SNPs with HDL-C, TG and TG:HDL-C ratio was first tested. No significant associations (p values ranged from 0.18 to 0.95) were found. Next, the interaction between each individual SNP and sex was tested and only the interaction between sex and the IVS5 SNP for TG was found to be significant (p=0.05) and borderline significant for the TG:HDL-C ratio (p=0.07) . the interaction between the three pair of SNPs in significant linkage disequilibrium was then tested and no evidence was found for association (p>0.21 for all interaction terms).

Two additional variants were genotyped in this population including a silent SNP in exon 7 (EX7) and a missense SNP in exon 3 (EX3). No women carried the EX3 variant and only two carried the EX7 variant. One had an HDL-C of 28 mg/dl, TG of 165 mg/dl and the other had an HDL-C of 39 mg/dl, TG of 132 mg/dl.

### Example 2

### Hormonal Influence on the Association of CD36L1 Genotypes with the TG:HDL-C Ratio

To test the hypothesis that hormones influence the association of CD36L1 genotypes with the TG:HDL-C ratio, we examined women from the GQ population stratified by menopause status and hormone use.

Among the 36 women from GQ who were premenopausal, significant interaction was found for the IVS5 and EX8 SNPs. Mean differences in TG:HDL-C ratio in this subpopulation by genotype combination were significant (p=0.001). Among the 29 post-menopausal women taking hormone replacement therapy, significant interaction was found again. Mean differences in TG:HDL-C ratio in this subpopulation by genotype combination were significant (p=0.06). However, among the 22 post-menopausal women not taking hormone replacement therapy, no association was found (p=0.31).

These results support the hypothesis that the association of lipids with CD36L1 variants is modulated by hormonal status.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method for determining whether a subject has, or is at risk of developing, abnormally high TG level or an abnormally high TG:HDL-C ratio, comprising determining whether the subject has an allelic variant of a polymorphic region of the CD36L1 gene that is associated with abnormally high TG levels or abnormally high TG:HDL-C ratios, to thereby determine whether the subject has, or is at risk of developing an abnormally high TG level or an abnormally high TG:HDL-C ratio.

2. The method of claim 1, wherein an abnormally high TG level or an abnormally high TG:HDL-C ratio is indicated by the presence of CT at IVS5 and CT at EX8, or the complements thereof, or TT at IVS5 and CT at EX8, or the complements thereof.

3. The method of claim 2, wherein determining the identity of the allelic variant of a polymorphic region comprises contacting a nucleic acid of the subject with at least one probe or primer which is capable of hybridizing to a CD36L1 gene.

4. A method of claim 3, wherein the probe or primer is capable of specifically hybridizing to an allelic variant of the polymorphic region.

5. A method of claim 1, wherein the probe or primer has a nucleotide sequence from about 15 to about 30 nucleotides.

6. A method of claim 1, wherein the probe or primer is a single stranded nucleic acid.

7. A method of claim 1, wherein the probe or primer is labeled.

8. A method of claim 1, wherein determining the identity of the allelic variant of a polymorphic region is carried out by allele specific hybridization.

9. A method of claim 1, wherein determining the identity of the allelic variant of a polymorphic region is carried out by primer specific extension.

10. A method of claim 1, wherein determining the identity of the allelic variant of a polymorphic region is carried out by an oligonucleotide ligation assay.

11. A method of claim 1, wherein determining the identity of the allelic variant of a polymorphic region is carried out by single-stranded conformation polymorphism.

12. A method of diagnosing or aiding in the diagnosis of abnormally high TG level or TG:HDL-C ratio in a subject comprising the steps of:
(a) obtaining a nucleic acid sample from the subject; and
(b) determining the identity of the nucleotides at nucleotide positions 41 of exon 8 and 54 of intron 5 of the CD36L1 gene, or the complements thereof,
wherein the presence of CT at IVS5 and CT at EX8, or the complements thereof, or TT at IVS5 and CT at EX8, or the complements thereof, is indicative of increased likelihood of abnormally high TG level or TG:HDL-C ratio in the subject as compared with a subject having any other combination of these alleles.

13. A method for treating a subject having a disease or disorder associated with specific allelic variants of a CD36L1 gene, comprising the steps of:
(a) determining the identity of CD36L1 allelic variants associated with an abnormally high TG level or TG:HDL-C ratio; and
(b) administering to the subject a compound that modulates CD36L1 gene expression or protein activity.

14. The method of claim 13, wherein the specific allelic variants are CT at IVS5 and CT at EX8 in combination, or the complements thereof, or TT at IVS5 and CT at EX8 in combination, or the complements thereof.

15. A kit for determining whether a subject has, or is at risk of developing, abnormally high TG level or an abnormally high TG:HDL-C ratio, comprising a probe or primer which is capable of hybridizing to a polymorphic region of a CD36L1 gene and thereby identifying whether the CD36L1 gene contains an allelic variant of a polymorphic region which is associated with abnormally high TG level or an abnormally high TG:HDL-C ratio, and instructions for use in diagnosing a subject as having, or is at risk of developing, towards developing abnormally high TG level or an abnormally high TG:HDL-C ratio.

16. The kit of claim 15, wherein an abnormally high TG level or an abnormally high TG:HDL-C ratio is indicated by the presence of CT at IVS5 and CT at EX8, or the complements thereof, or TT at IVS5 and CT at EX8, or the complements thereof.

17. A method for predicting the effect of hormone replacement therapy on the TG level or TG:HDL-C ratio in a female subject comprising identifying one or more allelic variants of the CD36L1 gene which are associated with abnormally high TG level or TG:HDL-C ratio in females, thereby predicting the effect of hormone replacement therapy on the TG level or TG:HDL-C ratio in the subject.

18. The method of claim 17, wherein presence of CT at IVS5 and CT at EX8, or the complements thereof, or TT at IVS5 and CT at EX8, or the complements thereof, indicates the effect of hormone replacement therapy in a subject to be an increase in TG level or TG:HDL-C ratio.

19. The method of claim 17, wherein the female subject is postmenopausal.

20. An Internet-based method for assessing a subject's risk for developing an abnormally high TG level or an abnormally high TG:HDL-C ratio, the method comprising:
a) analyzing biological information from a subject indicative of the presence or absence of a polymorphic region of CD36L1;
b) providing results of the analysis to the subject via the Internet, wherein the presence of a polymorphic region of CD36L1 indicates an increased risk for developing an abnormally high TG level or an abnormally high TG:HDL-C ratio.

21. A method of assessing a subject's risk for developing an abnormally high TG level or an abnormally high TG:HDL-C ratio, the method comprising:
a) obtaining biological information from the individual;
b) analyzing the information to obtain the subject's CD36L1 genetic profile;
c) representing the CD36L1 genetic profile information as digital genetic profile data;
d) electronically processing the CD36L1 digital genetic profile data to generate a risk assessment report for developing an abnormally high TG level or an abnormally high TG:HDL-C ratio, wherein the presence of a polymorphic region of CD36L1 indicates an increased risk for developing an abnormally high TG level or an abnormally high TG:HDL-C ratio; and
e) displaying the risk assessment report on an output device.

22. A method of assessing a subject's risk for developing an abnormally high TG level or an abnormally high TG:HDL-C ratio, the method comprising:
a) obtaining the subject's CD36L1 genetic profile information as digital genetic profile data;
b) electronically processing the CD36L1 digital genetic profile data to generate a risk assessment report for developing an abnormally high TG level or an abnormally high TG:HDL-C ratio, wherein the presence of a polymorphic region of CD36L1 indicates an increased risk for developing an abnormally high TG level or an abnormally high TG:HDL-C ratio; and
c) displaying the risk assessment report on an output device.

23. The method of claim 22, further comprising the step of using the risk assessment report to provide medical advice.

24. The method of claim 22, wherein the CD36L1 digital genetic profile data are transmitted via a communications network to a medical information system for processing.

25. The method of claim 24, wherein the communications network is the Internet.

26. A medical information system for assessing a subject's risk for developing an abnormally high TG level or an abnormally high TG:HDL-C ratio comprising:
a) means for obtaining biological information from the individual to obtain a CD36L1 genetic profile;
b) means for representing the CD36L1 genetic profile as digital molecular data;
c) means for electronically processing the CD36L1 digital genetic profile to generate a risk assessment report for developing an abnormally high TG level or an abnormally high TG:HDL-C ratio; and
d) means for displaying the risk assessment report on an output device, wherein the presence of a polymorphic region of CD36L1 indicates an increased risk for developing an abnormally high TG level or an abnormally high TG:HDL-C ratio.

27. A computerized method of providing medical advice to a subject comprising:
a) based on the subject's CD36L1 genetic profile, determining the subject's risk for developing an abnormally high TG level or an abnormally high TG:HDL-C ratio;
b) based on the subject's risk for vascular disease, electronically providing medical advice to the subject.

28. The method of claims 27, wherein the medical advice comprises one or more of the group consisting of further diagnostic evaluation, administration of medication, or lifestyle change.

29. A method of self-assessing risk for developing an abnormally high TG level or an abnormally high TG:HDL-C ratio, the method comprising accessing CD36L1 digital genetic profile data obtained from biological information, the CD36L1 digital genetic profile data being displayed via an output device, wherein the presence of a polymorphic region of CD36L1 indicates an increased risk for developing an abnormally high TG level or an abnormally high TG:HDL-C ratio.

30. The method of claim 29, wherein the electronic output device is accessed via the Internet.

31. The method of claim 29, wherein additional health information is provided.

32. The method of claim 31, wherein the additional health information comprises information regarding one or more of age, sex, ethnic origin, diet, sibling health, parental health, clinical symptoms, personal health history, blood test data, weight, and alcohol use, drug use, nicotine use, and blood pressure.

33. A method for a health care provider to generate a personal health assessment report for an individual, the method comprising counseling the individual to provide a biological sample; authorizing a draw station to take a biological sample from the individual and transmit molecular information from the sample to a laboratory company, wherein the molecular information comprises the presence or absence of a polymorphic region of CD36L1; requesting the laboratory company to provide digital molecular data corresponding to the molecular information to a medical information system to electronically process the digital molecular data and digital health data obtained from the individual to generate a health assessment report; receiving the health assessment report from the medical information system; and providing the health assessment report to the individual.

34. A method of assessing the health of an individual, the method comprising: obtaining health information from the individual using an input device; representing at least some of the health information as digital health data; obtaining biological information from the individual, wherein the information comprises the presence or absence of a polymorphic region of CD36L1; representing at least some of the information as digital molecular data; electronically processing the digital molecular data and digital health data to generate a health assessment report; and displaying the health assessment report on an output device.

35. The method of claim 34, wherein electronically processing the digital molecular data and digital health data to generate a health assessment report comprises using the digital molecular data and digital health data as inputs for an algorithm or a rule-based system that determines whether the individual is at risk for a specific disorder.

36. The method of claim 34, wherein the individual has or is at risk of developing an abnormally high TG level or an abnormally high TG:HDL-C ratio, and wherein electronically processing the digital molecular data and digital health data to generate a health assessment report comprises using the digital molecular data and digital health data as inputs for an algorithm or a rule-based system that determines the individual's prognosis.

37. The method of claim 34, wherein electronically processing the digital molecular data and digital health data comprises using the digital molecular data and digital health data as inputs for an algorithm or a rule-based system based on one or more databases comprising stored digital molecular data and/or digital health data relating to one or more disorders.

38. The method of claim 34, wherein electronically processing the digital molecular data and digital health data comprises using the digital molecular data and digital health data as inputs for an algorithm or a rule-based system based on one or more databases comprising (i) stored digital molecular data and/or digital health data from a plurality of healthy individuals, and (ii) stored digital molecular data and/or digital health data from one or more pluralities of unhealthy individuals, each plurality of individuals having a specific disorder.

39. The method of claim 38, wherein the communications network is the Internet.

40. The method of claim 38, wherein the input device is a keyboard, touch screen, hand-held device, telephone, wireless input device, or interactive page on a website.

41. The method of claim 34, wherein the health assessment report comprises a digital molecular profile of the individual.

42. The method of claim 34, wherein the health assessment report comprises a digital health profile of the individual.

43. The method of claim 34, wherein the molecular data comprises nucleic acid sequence data, and the molecular profile comprises a genetic profile.

44. The method of claim 34, wherein the health information comprises information relating to one or more of age, sex, ethnic origin, diet, sibling health, parental health, clinical symptoms, personal health history, blood test data, weight, and alcohol use, drug use, nicotine use, and blood pressure.

45. The method of claim 34, further comprising obtaining a second set of biological information at a time after obtaining the first set of biological information; processing the second set of biological information to obtain a second set of information; representing at least some of the second set of information as digital second molecular data; and processing the molecular data and second molecular data to generate a health assessment report.

46. The method of claim 34, wherein the health assessment report provides information about the individual's predisposition for developing an abnormally high TG level or an abnormally high TG:HDL-C ratio and options for risk reduction.

47. The method of claim 46, wherein the options for risk reduction comprise one or more of diet, exercise, one or more vitamins, one or more drugs, cessation of nicotine use, and cessation of alcohol use.

48. The method of claim 47, further comprising building a database of stored molecular data from a plurality of individuals.

49. The method of claim 34, further comprising storing the molecular data and health data.

50. The method of claim 49, further comprising building a database of stored molecular data and health data from a plurality of individuals.

51. The method of claim 49, further comprising building a database of stored digital molecular data and/or digital health data from a plurality of healthy individuals, and stored digital molecular data and/or digital health data from one or more pluralities of unhealthy individuals, each plurality of individuals having a specific disorder.

52. The method of claim 51, further comprising building a database of stored molecular data and health data from a plurality of individuals.


SEQUENCE LISTING (121 sequences)

SEQ ID NO: 1 - DNA, 2630 bp, Homo sapiens
  CDS (119)..(1645)
accgtgcctctgcggcctgcgtgcccggagtccccgcctgtgtcgtctctgtcgccgtccccgtctcctgccaggcgcggagccctgcgagccgcgggtgggccccaggcgcgcagacatgggctgctccgccaaagcgcgctgggctgccggggcgctgggcgtcgcggggctactgtgcgctgtgctgggcgctgtcatgatcgtgatggtgccgtcgctcatcaagcagcaggtccttaagaacgtgcgcatcgaccccagtagcctgtccttcaacatgtggaaggagatccctatccccttctatctctccgtctacttctttgacgtcatgaaccccagcgagatcctgaagggcgagaagccgcaggtgcgggagcgcgggccctacgtgtacagggagttcaggcacaaaagcaacatcaccttcaacaacaacgacaccgtgtccttcctcgagtaccgcaccttccagttccagccctccaagtcccacggctcggagagcgactacatcgtcatgcccaacatcctggtcttgggtgcggcggtgatgatggagaataagcccatgaccctgaagctcatcatgaccttggcattcaccaccctcggcgaacgtgccttcatgaaccgcactgtgggtgagatcatgtggggctacaaggacccccttgtgaatctcatcaacaagtactttccaggcatgttccccttcaaggacaagttcggattatttgctgagctcaacaactccgactctgggctcttcacggtgttcacgggggtccagaacatcagcaggatccacctcgtggacaagtggaacgggctgagcaaggttgacttctggcattccgatcagtgcaacatgatcaatggaacttctgggcaaatgtggccgcccttcatgactcctgagtcctcgctggagttctacagcccggaggcctgccgatccatgaagctaatgtacaaggagtcaggggtgtttgaaggcatccccacctatcgcttcgtggctcccaaaaccctgtttgccaacgggtccatctacccacccaacgaaggcttctgcccgtgcctggagtctggaattcagaacgtcagcacctgcaggttcagtgcccccttgtttctctcccatcctcacttcctcaacgccgacccggttctggcagaagcggtgactggcctgcaccctaaccaggaggcacactccttgttcctggacatccacccggtcacgggaatccccatgaactgctctgtgaaactgcagctgagcctctacatgaaatctgtcgcaggcattggacaaactgggaagattgagcctgtggtcctgccgctgctctggtttgcagagagcggggccatggagggggagactcttcacacattctacactcagctggtgttgatgcccaaggtgatgcactatgcccagtacgtcctcctggcgctgggctgcgtcctgctgctggtccctgtcatctgccaaatccggagccaagagaaatgctatttattttggagtagtagtaaaaagggctcaaaggataaggaggccattcaggcctattctgaatccctgatgacatcagctcccaagggctctgtgctgcaggaagcaaaactgtagggtcctgaggacaccgtgagccagccaggcctggccgctgggcctgaccggccccccagcccctacaccccgcttctcccggactctcccagcagacagccccccagccccacagcctgagcctcccagctgccatgtgcctgttgcacacctgcacacacgccctggcacacatacacacatgcgtgcaggcttgtgcagacactcagggatggagctgctgctgaagggacttgtagggagaggctcgtcaacaagcactgttctggaaccttctctccacgtggcccacaggcctgaccacaggggctgtgggtcctgcgtccccttcctcgggtgagcctggcctgtcccgttcagccgttgggcccaggcttcctcccctccaaggtgaaacactgcagtcccggtgtggtggctccccatgcaggacgggccaggctgggagtgccgccttcctgtgccaaattcagtggggactcagtgcccaggccctggccacgagctttggccttggtctacctgccaggccaggcaaagcgcctttacacaggcctcggaaaacaatggagtgagcacaagatgccctgtgcagctgcccgagggtctccgcccaccccggccggactttgatccccccgaagtcttcacaggcactgcatcgggttgtctggcgcccttttcctccagcctaaactgacatcatcctatggactgagccggccactytytggccgaagtggccgcaggctgtgcccccgagctgcccccaccccctcacagggtccctcagattataggtgcccaggctgaggtgaagaggcctgggggccctgccttccgggcgctcctggaccctggggcaaacctgtgacccttttctactggaatagaaatgagttttatcatctttgaaaaataattcactcttgaagtaataaacgtttaaaaaaatgggaaaaaaaaaaaaaaaaaaaaaaaa

SEQ ID NO: 2 - protein, 509 aa, Homo sapiens
MGCSAKARWAAGALGVAGLLCAVLGAVMIVMVPSLIKQQVLKNVRIDPSSLSFNMWKEIPIPFYLSVYFFDVMNPSEILKGEKPQVRERGPYVYREFRHKSNITFNNNDTVSFLEYRTFQFQPSKSHGSESDYIVMPNILVLGAAVMMENKPMTLKLIMTLAFTTLGERAFMNRTVGEIMWGYKDPLVNLINKYFPGMFPFKDKFGLFAELNNSDSGLFTVFTGVQNISRIHLVDKWNGLSKVDFWHSDQCNMINGTSGQMWPPFMTPESSLEFYSPEACRSMKLMYKESGVFEGIPTYRFVAPKTLFANGSIYPPNEGFCPCLESGIQNVSTCRFSAPLFLSHPHFLNADPVLAEAVTGLHPNQEAHSLFLDIHPVTGIPMNCSVKLQLSLYMKSVAGIGQTGKIEPVVLPLLWFAESGAMEGETLHTFYTQLVLMPKVMHYAQYVLLALGCVLLLVPVICQIRSQEKCYLFWSSSKKGSKDKEAIQAYSESLMTSAPKGSVLQEAKL

SEQ ID NO: 3 - DNA, 1825 bp, Homo sapiens
  CDS (156)..(1682)
gccacctgcagggctactgctgctccggccactgcctgagactcaccttgctggaacgtgagcctcggcttctgtcatctctgtggcctctgtcgcttctgtcgctgtcccccttcagtccctgagccccgcgagcccgggccgcacacgcggacatgggcggcagcgccagggcgcgctgggtggcggtggggctgggcgtcgtggggctgctgtgcgctgtgctcggtgtggttatgatcctcgtgatgccctcgctcatcaaacagcaggtactgaagaatgtccgcatagaccccagcagcctgtcctttgcaatgtggaaggagatccctgtacccttctacttgtccgtctacttcttcgaggtggtcaatcccagcgagatcctaaagggtgagaagccagtagtgcgggagcgtggaccctatgtctacagggaattcagacataaggccaacatcaccttcaatgacaatgatactgtgtcctttgtggagcaccgcagcctccatttccagccggacaggtcccacggctctgagagtgactacattatactgcctaacattctggtcttggggggcgcagtaatgatggagagcaagtctgcaggcctgaagctgatgatgaccttggggctggccaccttgggccagcgtgcctttatgaaccgaacagttggtgagatcctgtggggctatgaggatcccttcgtgaattttatcaacaaatacttaccagacatgttccccatcaagggcaagttcggcctgtttgttgagatgaacaactcagactctgggctcttcactgtgttcacgggcgtccagaacttcagcaagatccacctggtggacagatggaatgggctcagcaaggtcaactactggcattcagagcagtgcaacatgatcaatggcacttccgggcagatgtgggcaccattcatgacaccccagtcctcgctggaattcttcagtccggaagcctgcaggtctatgaagctcacctaccatgattcaggggtgtttgaaggcatccccacctatcgcttcacagcccctaaaactttgtttgccaatgggtctgtttacccacccaatgaaggtttctgcccgtgccttgaatccggcattcaaaatgtcagcacttgcaggtttggtgcacccctgtttctgtcacaccctcacttctacaatgcagaccctgtgctatcagaagccgttctgggtctgaaccctgacccaagggagcattctttgttccttgacatccatccggtcactgggatccccatgaactgttctgtgaagttgcagataagcctctacatcaaagctgtcaagggcattgggcaaacagggaagatcgagcccgtggtcctcccattgctgtggtttgagcagagcggtgccatgggcggcgagcccctgaacacgttctacacgcagctggtgctgatgccccaggtacttcagtatgtgcagtatgtgctgctggggctgggcggcctcctgctgctggtgcccgtcatctaccagttgcgcagccaggagaaatgctttttattttggagtggtagtaaaaagggctcgcaggataaggaggccattcaggcctactctgagtctctgatgtcaccagctgccaagggcacggtgctgcaagaagccaagctgtagggtcccaaagacaccacgagcccccccaacctgatagcttggtcagaccagccatccagcccctacaccccgcttcttgaggactctctcagcggacagtccgccagtgccatggcctgagccccagatgtcacacctgt

SEQ ID NO: 4 - protein, 509 aa, Homo sapiens
MGGSARARWVAVGLGVVGLLCAVLGVVMILVMPSLIKQQVLKNVRIDPSSLSFAMWKEIPVPFYLSVYFFEVVNPSEILKGEKPVVRERGPYVYREFRHKANITFNDNDTVSFVEHRSLHFQPDRSHGSESDYIILPNILVLGGAVMMESKSAGLKLMMTLGLATLGQRAFMNRTVGEILWGYEDPFVNFINKYLPDMFPIKGKFGLFVEMNNSDSGLFTVFTGVQNFSKIHLVDRWNGLSKVNYWHSEQCNMINGTSGQMWAPFMTPQSSLEFFSPEACRSMKLTYHDSGVFEGIPTYRFTAPKTLFANGSVYPPNEGFCPCLESGIQNVSTCRFGAPLFLSHPHFYNADPVLSEAVLGLNPDPREHSLFLDIHPVTGIPMNCSVKLQISLYIKAVKGIGQTGKIEPVVLPLLWFEQSGAMGGEPLNTFYTQLVLMPQVLQYVQYVLLGLGGLLLLVPVIYQLRSQEKCFLFWSGSKKGSQDKEAIQAYSESLMSPAAKGTVLQEAKL

SEQ ID NO: 5 - DNA, 1002 bp, Homo sapiens
actgcggagatgagggtctagaaggtggtggcggggcatgtggaccgttgtaagggctctggggttcctgggtgggctggcgaagtcctactcacagtgaccaaccatgatgatggtcccgatagaggaggagagggaggaggagggaaaaggaagggtgaggggctcagaggggagagctgggaggaggggagacataggtgggggaaggggtaggagaaaggggaagggagcaagagggtgaggggcaccaggccccatagacgttttggctcagcggccacgaggcttcatcagctcccgccccaaaacggaagcgaggccgtgggggcagcggcagcatggcggggcttgtcttggcggccatggccccgccccctgcccgtccgatcagcgccccgccccgtccccgccccgaccccgccccgggcccgctcaggccccgcccctgccgccggaatcctgaagcccaaggctgcccgggggcggtccggcggcgccggcgatggggcataaaaccactggccacctgccgggctgctcctgcgtgcgctgccgtcccggatccaccgtgcctctgcggcctgcgtgccccgagtccccgcctgtgtcgtctctgtcgccgtccccgtctcctgccaggcgcggagccctgcgagccgcgggtgggccccaggcgcgcagacatgggctgctccgccaaagcgcgctgggctgccggggcgctgggcgtcgcggggctactgtgcgctgtgctgggcgctgtcatgatcgtgatggtgccgtcgctcatcaagcagcaggtccttaaggtgggtgagggagaccccagggggtccgcgcacggacccgggctgttgggcgctgggcgccgggaggacccgcgcgttgcggtgggtgggcgaccgcagcggaatcggcgcccgggcctggcgccgcagaacacgagggaggccaggcgcttcgggaggggctgctgcccgcctccccaccaccctcacc

SEQ ID NO: 6 - DNA, 479 bp, Homo sapiens
agcctcatgtgcgaagggctttcccaccacctcctatcccaagctcccgccgaggagccccttccctggccgggctcgggcagctgttccggagccttgtggtggggcgtggggccctcatcactctcctcacaagcgtacttgtcccttcccctgcagaacgtgcgcatcgaccccagtagcctgtccttcaacatgtggaaggagatccctatccccttctatctctccgtctacttctttgacgtcatgaaccccagcgagatcctgaagggcgagaagccgcaggtgcgggagcgcgggccctacgtgtacaggtgaggctgtgtccacgtgatggtggacgggccggctgacgctgggcatgggacgggtctcaagtggacgggatggggaggctgctgactgacccccaaacattgttccggaagcacgcaactcatagtcggggtaagtgctactcccaaaaaagtttgcgt

SEQ ID NO: 7 - DNA, 495 bp, Homo sapiens
catgtcctgcagtgggcaggcagcgggagggacagacttggcgaaggggccgagctcagctttggctgtggggccggaggtgtgcacagacgtccagggcccctggttcccaggcaggcattgcaggcgagtagaagggaaacgtcccatgcagcggggcggggcgtctgacccactggcttcccccacagggagttcaggcacaaaagcaacatcaccttcaacaacaacgacaccgtgtccttcctcgagtaccgcaccttccagttccagccctccaagtcccacggctcggagagcgactacatcgtcatgcccaacatcctggtcttggtgaggctgccctgtggcccacgccgcctcgcaccctgacctcgtcccctgtctctcctcccgcctgccccttgtgcagagagcagtccctgaggtggtcggagcgtggggactcacgcctggtgggtggctttcggccctgtgctgtctccaccaccccca

SEQ ID NO: 8 - DNA, 526 bp, Homo sapiens
ggtggttctggtgtcccagatgccccacgtggccactccaggggcctcctgcaccccagcatttcccttcatgggctctttgctgtgaggcccagctggggccaagggaggatgggccagccacgtccagcctctgacactagtgtcccttcgccttgcagggtgcggcggtgatgatggagaataagcccatgaccctgaagctcatcatgaccttggcattcaccaccctcggcgaacgtgccttcatgaaccgcactgtgggtgagatcatgtggggctacaaggaccccttgtgaatctcatcaacaagtactttccaggcatgttccccttcaaggacaagttcggattatttgctgaggtacgtgtggcctggtgagaagccaaagattcaggcctgtgtcctgtcttcccctcacacagcctggacactggtcaccagcttgctttgtagctggctggggatctagtggctgtgggttgtaagtgactgagaacctgactcaaaccggcttgagtgaaa

SEQ ID NO: 9 - DNA, 416 bp, Homo sapiens
cctctcggtccccagacactgggcatttggcagtgaaccagatgctgggggccctgtccttctggtggagggggaggagggctcagcccagaatgttcagaccaggccggctcaatggcaggcctaagccttacgatgctgttccctgctgtgtctgtagctcaacaactccgactctgggctcttcacggtgttcacgggggtccagaacatcagcaggatccacctcgtggacaagtggaacgggctgagcaaggtgaggggcgagaggcgagggcccctgtcgccagggagaggggagggtgggcccggccatggctgctcgggagtggcagggaccagagagctccttcttcctttgtcgtgaagagggtgctgggaggatgaacactcttgaagttggaggagggatttta

SEQ ID NO: 10 - DNA, 436 bp, Homo sapiens
tctctgtgtgtctacatagcctgccctcttcccaccgtgccagtattgggaattgagtggccgtgcgtgcaccagggtgagttaggtgtgcagcacctgagagggcttattaaggggccttggccctactgaggggtctagtctggatgcttccccccaggttgacttctggcattccgatcagtgcaacatgatcaatggaacttctgggcaaatgtggccgcccttcatgactcctgagtcctcgctggagttctacagcccggaggcctgccggtaatcactgggactcggggcctcctgggtttcctgggtagctcatggccaaattctgtggtgttggctgtgcacttggaaagcattttgactcatcgtggatttgactcagtagcccttggcaccagcttgaattctctttggtcacaccaccaaaagc

SEQ ID NO: 11 - DNA, 481 bp, Homo sapiens
  misc_feature 355, 373, 375, 384, 386: n = A,T,C or G
ggaggtcgctgcagctccgcgggtgagagatgggggcggtttggacccgggaggtggtagcgcccgtggggagaagtggctggatctgggcagcctttggcagggcctggctctggccgccgggtctgggtgtcccctctcatcctgtctgtcccctgcagatccatgaagctaatgtacaaggagtcaggggtgtttgaaggcatccccacctatcgcttcgtggctcccaaaaccctgtttgccaacgggtccatctacccacccaacgaaggcttctgcccgtgcctggagtctggaattcagaacgtcagcagctgcaggttcagtacgtgccgtcccctgttctgggatngccggagggtgttaggtntngggcacctnanggtttatctgcccaatgctgtctgcttaatctctggcctctgtactcttgataacccattaagccaaaaatatgatgcctctgggacgatatctg

SEQ ID NO: 12 - DNA, 430 bp, Homo sapiens
tggggctttttacagaatggaggaagggatcctctctgtcgggtattatggtcatcgccacgggggtgccgtgcagaccacagctctgtgcagacttccggagtggcaggacgtgccaatatactgtcgttgtatgatgtcccctccctgcccttgttgtaggtgcccccttgtttctctcccatcctcacttcatcaacgccgacccggttctggcagaagcggtgactggcctgcaccctaaccaggaggcacactccttgttcgtggacatccacccggtgagcccctgccatcctctgtggggggtgggtgattcctggttggagcacacctggctgcctcctctctccccaggcagagagctgctgtgggctggggtggtgggaagcctggcttctagaatctcgagccaccaaagttccttact

SEQ ID NO: 13 - DNA, 390 bp, Homo sapiens
  misc_feature 279: n = A,T,C or G
ccccagcctgtggcttgttttaggtaagatacaagcaagctccactgggcagttagctgggacgcccaccctcttgactgggaccagggaaaagaaggttgactgtgtccctggagcttgggggtggccagtctcctcactgtgtttgttgccgcaggtcacgggaatccccatgaactgctctgtgaaactgcagctgagcctctacatgaaatctgtcgcaggcattgggtgagtggggactgggaactggggctgcattgctcattgagagattangtgctcagtgctccagtgttcccagactcccctgacataccccaggaaacagggcatggggaagggagagggtcctattgggggtggaatccagtccctgctgatcttctc

SEQ ID NO: 14 - DNA, 370 bp, Homo sapiens
atggctcctaaagtgtttcagctcattgtttatatttggtggtgagggtttagtgtgtgcaaaattatactaaacctgtttagatgttgtattcaagcagaattagatcaagtttgggtgtaagactttgttccaacacctatgtcttgcttatttccagacaaactgggaagattgagcctgtggtcctgccgctgctctggtttgcagaggtaagggtgcgttgggcacagcgtcgggggcttttgttaatagccaatgtgggcatttgaggcaggaggcggggggagcaccttgtagaaagggagagggctgagccagggtaaccggactgttacatggaccagcgtatcatacacttcaccctgtc

SEQ ID NO: 15 - DNA, 470 bp, Homo sapiens
cctggagggaggaggtccctggcaggctccaacacatgctttagccgggaagcttgaggtggggaaaagctgaggcgggcacagaggaaggtgttgggtggcatctgcgctgtagcccgcagcctgcggccccagctcatgtgtttgtcattctgtctcctcagagcggggccatggagggggagactcttcacacattctacactcagctggtgttgatgcccaaggtgatgcactatgcccagtacgtcctcctggcgctgggctgcgtcctgctgctggtccctgtcatctgccaaatccggagccaagtaggtgctggccagagggcagcccgggctgacagccattcgcttgcctgctgggggaaaggggcctcagatcggaccctctggccaaccgcagcctggagcccacctccagcagcagtcctgcgtctctgccggagtgggagcggtcactgctggggg

SEQ ID NO: 16 - DNA, 450 bp, Homo sapiens
ccccacatctcagccacctgcaatcgttgagggttgttggactctaaacttatgtgcctttcctgtttcctctttgccttttgcaaattgaagaaccgtgtaaaaccatttttatgtggcttcaacgtcaactataaattagcttggttatcttctaggagaaatgctatttattttggagtagtagtaaaaagggctcaaaggataaggaggccattcaggcctattctgaatccctgatgacatcagctcccaagggctctgtgctgcaggaagcaaaactgtaggtgggtaccaggtaatgccgtgcgcctccccgccccctcccatatcaagtagaatgctggcggcttaaaacatttggggtcctgctcattccttcagcctcaacttcacctggagtgtctacagactgaagatgcatatttgtgtattttgcttttggagaaa

SEQ ID NO: 17 - DNA, 544 bp, Homo sapiens
actgcggagatgagggtctagaaggtggtggcggggcatgtggaccgttgtaagggctctggggttcctgggtgggctggcgaagtcctactcacagtgaccaaccatgatgatggtcccgatagaggaggagagggaggaggagggaaaaggaagggtgaggggctcagaggggagagctgggaggaggggagacataggtgggggaaggggtaggagaaaggggaagggagcaagagggtgaggggcaccaggccccatagacgttttggctcagcggccacgaggcttcatcagctcccgccccaaaacggaagcgaggccgtgggggcagcggcagcatggcggggcttgtcttggcggccatggccccgccccctgcccgtccgatcagcgccccgccccgtccccgccccgaccccgccccgggcccgctcaggccccgcccctgccgccggaatcctgaagcccaaggctgcccgggggcggtccggcggcgccggcgatggggcataaaaccactggccacctgccgggctgctcc

SEQ ID NO: 18 - DNA, 190 bp, Homo sapiens
gtgggtgagggagaccccagggggtccgcgcacggacccgggctgttgggcgctgggcgccgggaggacccgcgcgttgcggtgggtgggcgaccgcagcggaatcggcgcccgggcctggcgccgcagaacacgagggaggccaggcgcttcgggaggggctgctgcccgcctccccaccaccctcacc

SEQ ID NO: 19 - DNA, 159 bp, Homo sapiens
agcctcatgtgcgaagggctttcccaccacctcctatcccaagctcccgccgaggagccccttccctggccgggctcgggcagctgttccggagccttgtggtggggcgtggggccctcatcactctcctcacaagcgtacttgtcccttcccctgcag

SEQ ID NO: 20 - DNA, 162 bp, Homo sapiens
gtgaggctgtgtccacgtgatggtggacgggccggctgacgctgggcatgggacgggtctcaagtggacgggatggggaggctgctgactgacccccaaacattgttccggaagcacgcaactcatagtcggggtaagtgctactcccaaaaaagtttgcgt

SEQ ID NO: 21 - DNA, 191 bp, Homo sapiens
catgtcctgcagtgggcaggcagcgggagggacagacttggcgaaggggccgagctcagctttggctgtggggccggaggtgtgcacagacgtccagggcccctggttcccaggcaggcattgcaggcgagtagaagggaaacgtcccatgcagcggggcggggcgtctgacccactggcttcccccacag

SEQ ID NO: 22 - DNA, 162 bp, Homo sapiens
gtgaggctgccctgtggcccacgccgcctcgcaccctgacctcgtcccctgtctctcctcccgcctgccccttgtgcagagagcagtccctgaggtggtcggagcgtggggactcacgcctggtgggtggctttcggccctgtgctgtctccaccaccccca

SEQ ID NO: 23 - DNA, 161 bp, Homo sapiens
ggtggttctggtgtcccagatgccccacgtggccactccaggggcctcctgcaccccagcatttcccttcatgggctctttgctgtgaggcccagctggggccaagggaggatgggccagccacgtccagcctctgacactagtgtcccttcgccttgcag

SEQ ID NO: 24 - DNA, 162 bp, Homo sapiens
gtacgtgtggcctggtgagaagccaaagattcaggcctgtgtcctgtcttcccctcacacagcctggacactggtcaccagcttgctttgtagctggctggggatctagtggctgtgggttgtaagtgactgagaacctgactcaaaccggcttgagtgaaa

SEQ ID NO: 25 - DNA, 160 bp, Homo sapiens
cctctcggtccccagacactgggcatttggcagtgaaccagatgctgggggccctgtccttctggtggagggggaggagggctcagcccagaatgttcagaccaggccggctcaatggcaggcctaagccttacgatgctgttccctgctgtgtctgtag

SEQ ID NO: 26 - DNA, 160 bp, Homo sapiens
gtgaggggcgagaggcgagggcccctgtcgccagggagaggggagggtgggcccggccatggctgctcgggagtggcagggaccagagagctccttcttcctttgtcgtgaagagggtgctgggaggatgaacactcttgaagttggaggagggatttta

SEQ ID NO: 27 - DNA, 160 bp, Homo sapiens
tctctgtgtgtctacatagcctgccctcttcccaccgtgccagtattgggaattgagtggccgtgcgtgcaccagggtgagttaggtgtgcagcacctgagagggcttattaaggggccttggccctactgaggggtctagtctggatgcttccccccag

SEQ ID NO: 28 - DNA, 160 bp, Homo sapiens
gtaatcactgggactcggggcctcctgggtttcctgggtagctcatggccaaattctgtggtgttggctgtgcacttggaaagcattttgactcatcgtggatttgactcagtagcccttggcaccagcttgaattctctttggtcacaccaccaaaagc

SEQ ID NO: 29 - DNA, 161 bp, Homo sapiens
ggaggtcgctgcagctccgcgggtgagagatgggggcggtttggacccgggaggtggtagcgcccgtggggagaagtggctggatctgggcagcctttggcagggcctggctctggccgccgggtctgggtgtcccctctcatcctgtctgtcccctgcag

SEQ ID NO: 30 - DNA, 153 bp, Homo sapiens
  misc_feature 27, 45, 47, 56, 58: n = A,T,C or G
gtacgtgccgtcccctgttctgggatngccggagggtgttaggtntngggcacctnanggtttatctgcccaatgctgtctgcttaatctctggcctctgtactcttgataacccattaagccaaaaatatgatgcctctgggacgatatctg

SEQ ID NO: 31 - DNA, 162 bp, Homo sapiens
tggggctttttacagaatggaggaagggatcctctctgtcgggtattatggtcatcgccacgggggtgccgtgcagaccacagctctgtgcagacttccggagtggcaggacgtgccaatatactgtcgttgtatgatgtcccctccctgcccttgttgtag

SEQ ID NO: 32 - DNA, 149 bp, Homo sapiens
gtgagcccctgccatcctctgtggggggtgggtgattcctggttggagcacacctggctgcctcctctctccccaggcagagagctgctgtgggctggggtggtgggaagcctggcttctagaatctcgagccaccaaagttccttact

SEQ ID NO: 33 - DNA, 157 bp, Homo sapiens
ccccagcctgtggcttgttttaggtaagatacaagcaagctccactgggcagttagctgggacgcccaccctcttgactgggaccagggaaaagaaggttgactgtgtccctggagcttgggggtggccagtctcctcactgtgtttgttgccgcag

SEQ ID NO: 34 - DNA, 159 bp, Homo sapiens
  misc_feature 48: n = A,T,C or G
gtgagtggggactgggaactggggctgcattgctcattgagagattangtgctcagtgctccagtgttcccagactcccctgacataccccaggaaacagggcatggggaagggagagggtcctattgggggtggaatccagtccctgctgatcttctc

SEQ ID NO: 35 - DNA, 160 bp, Homo sapiens
atggctcctaaagtgtttcagctcattgtttatatttggtggtgagggtttagtgtgtgcaaaattatactaaacctgtttagatgttgtattcaagcagaattagatcaagtttgggtgtaagactttgttccaacacctatgtcttgcttatttccag

SEQ ID NO: 36 - DNA, 158 bp, Homo sapiens
gtaagggtgcgttgggcacagcgtcgggggcttttgttaatagccaatgtgggcatttgaggcaggaggcggggggagcaccttgtagaaagggagagggctgagccagggtaaccggactgttacatggaccagcgtatcatacacttcaccctgtc

SEQ ID NO: 37 - DNA, 164 bp, Homo sapiens
cctggagggaggaggtccctggcaggctccaacacatgctttagccgggaagcttgaggtggggaaaagctgaggcgggcacagaggaaggtgttgggtggcatctgcgctgtagcccgcagcctgcggccccagctcatgtgtttgtcattctgtctcctcag

SEQ ID NO: 38 - DNA, 159 bp, Homo sapiens
gtaggtgctggccagagggcagcccgggctgacagccattcgcttgcctgctgggggaaaggggcctcagatcggaccctctggccaaccgcagcctggagcccacctccagcagcagtcctgcgtctctgccggagtgggagcggtcactgctggggg

SEQ ID NO: 39 - DNA, 158 bp, Homo sapiens
ccccacatctcagccacctgcaatcgttgagggttgttggactctaaacttatgtgcctttcctgtttcctctttgccttttgcaaattgaagaaccgtgtaaaaccatttttatgtggcttcaacgtcaactataaattagcttggttatcttctag

SEQ ID NO: 40 - DNA, 163 bp, Homo sapiens
gtgggtaccaggtaatgccgtgcgcctccccgccccctcccatatcaagtagaatgctggcggcttaaaacatttggggtcctgctcattccttcagcctcaacttcacctggagtgtctacagactgaagatgcatatttgtgtattttgcttttggagaaa

SEQ ID NO: 41 - DNA, 23 bp, Homo sapiens
cccctgccgccggaatcctgaag

SEQ ID NO: 42 - DNA, 24 bp, Homo sapiens
cgctttggcggagcagcccatgtc

SEQ ID NO: 43 - DNA, 24 bp, Homo sapiens
tggggccctcatcactctcctcac

SEQ ID NO: 44 - DNA, 23 bp, Homo sapiens
gcagcctccccatcccgtccact

SEQ ID NO: 45 - DNA, 18 bp, Homo sapiens
attgcaggcgagtagaag

SEQ ID NO: 46 - DNA, 18 bp, Homo sapiens
caggcgggaggagagaca

SEQ ID NO: 47 - DNA, 20 bp, Homo sapiens
tgggctctttgctgtgaggc

SEQ ID NO: 48 - DNA, 20 bp, Homo sapiens
ccaggctgtgtgaggggaag

SEQ ID NO: 49 - DNA, 20 bp, Homo sapiens
gcccagaatgttcagaccag

SEQ ID NO: 50 - DNA, 20 bp, Homo sapiens
gcaccctcttcacgacaaag

SEQ ID NO: 51 - DNA, 19 bp, Homo sapiens
cacctgagagggcttatta

SEQ ID NO: 52 - DNA, 19 bp, Homo sapiens
caaaatgctttccaagtgc

SEQ ID NO: 53 - DNA, 20 bp, Homo sapiens
gccgccgggtctgggtgtcc

SEQ ID NO: 54 - DNA, 23 bp, Homo sapiens
cagaggccagagattaagcagac

SEQ ID NO: 55 - DNA, 20 bp, Homo sapiens
ttgtatgatgtcccctccct

SEQ ID NO: 56 - DNA, 20 bp, Homo sapiens
ttcccaccaccccagcccac

SEQ ID NO: 57 - DNA, 20 bp, Homo sapiens
ggttgactgtgtccctggag

SEQ ID NO: 58 - DNA, 21 bp, Homo sapiens
gggaacactggagcactgagc

SEQ ID NO: 59 - DNA, 20 bp, Homo sapiens
ggtggtgagggtttagtgtg

SEQ ID NO: 60 - DNA, 20 bp, Homo sapiens
ctccccccgcctcctgcctc

SEQ ID NO: 61 - DNA, 20 bp, Homo sapiens
aaggtgttgggtggcatctg

SEQ ID NO: 62 - DNA, 20 bp, Homo sapiens
ggctccaggctgcggttggc

SEQ ID NO: 63 - DNA, 19 bp, Homo sapiens
ttgaagaaccgtgtaaaac

SEQ ID NO: 64 - DNA, 18 bp, Homo sapiens
ttgaggctgaaggaatga

SEQ ID NO: 65 - DNA, 430 bp, Homo sapiens
tggggctttttacagaatggaggaagggatcctctctgtcgggtattatggtcatcgccacgggggtgccgtgcagaccacagctctgtgcagacttccggagtggcaggacgtgccaatatactgtcgttgtatgatgtcccctccctgcccttgttgtaggtgcccccttgtttctctcccatcctcacttcatcaacgctgacccggttctggcagaagcggtgactggcctgcaccctaaccaggaggcacactccttgttcgtggacatccacccggtgagcccctgccatcctctgtggggggtgggtgattcctggttggagcacacctggctgcctcctctctccccaggcagagagctgctgtgggctggggtggtgggaagcctggcttctagaatctcgagccaccaaagttccttact

SEQ ID NO: 66 - DNA, 160 bp, Homo sapiens
gtgaggggcgagaggcgagggcccctgtcgccagggagaggggagggtgggcctggccatggctgctcgggagtggcagggaccagagagctccttcttcctttgtcgtgaagagggtgctgggaggatgaacactcttgaagttggaggagggatttta

SEQ ID NO: 67 - DNA, 20 bp, Homo sapiens
aaccgggtcagcgttgagga

SEQ ID NO: 68 - DNA, 31 bp, Homo sapiens
tgccagaaccgggtcagcgttgaggaagtga

SEQ ID NO: 69 - DNA, 20 bp, Homo sapiens
tcctcaacgctgacccggtt

SEQ ID NO: 70 - DNA, 31 bp, Homo sapiens
tcacttcctcaacgctgacccggttctggca

SEQ ID NO: 71 - DNA, 20 bp, Homo sapiens
aaccgggtcggcgttgatga

SEQ ID NO: 72 - DNA, 31 bp, Homo sapiens
tgccagaaccgggtcggcgttgatgaagtga

SEQ ID NO: 73 - DNA, 20 bp, Homo sapiens
tcatcaacgccgacccggtt

SEQ ID NO: 74 - DNA, 31 bp, Homo sapiens
tcacttcatcaacgccgacccggttctggca

SEQ ID NO: 75 - DNA, 21 bp, Homo sapiens
agccatggccgggcccaccct

SEQ ID NO: 76 - DNA, 31 bp, Homo sapiens
cgagcagccatggccgggcccaccctcccct

SEQ ID NO: 77 - DNA, 21 bp, Homo sapiens
agggtgggcccggccatggct

SEQ ID NO: 78 - DNA, 31 bp, Homo sapiens
aggggagggtgggcccggccatggctgctcg

SEQ ID NO: 79 - DNA, 21 bp, Homo sapiens
agccatggccaggcccaccct

SEQ ID NO: 80 - DNA, 31 bp, Homo sapiens
cgagcagccatggccaggcccaccctcccct

SEQ ID NO: 81 - DNA, 21 bp, Homo sapiens
agggtgggcctggccatggct

SEQ ID NO: 82 - DNA, 31 bp, Homo sapiens
aggggagggtgggcctggccatggctgctcg

SEQ ID NO: 83 - DNA, 22 bp, Homo sapiens
tcctgggtgggctggcgaagtc

SEQ ID NO: 84 - DNA, 24 bp, Homo sapiens
gttttggggcgggagctgatgaag

SEQ ID NO: 85 - DNA, 18 bp, Homo sapiens
tgtaaaacgacggccagt

SEQ ID NO: 86 - DNA, 18 bp, Homo sapiens
caggaaacagctatgacc

SEQ ID NO: 87 - DNA, 62 bp, Homo sapiens
ctgagcaaggtgaggggcgagaggcgagggcccctgtcgccagggaggggagggtgggccyg

SEQ ID NO: 88 - DNA, 51 bp, Homo sapiens
cstgcggccccagctcatgtgtttgtcattctgtctcctcagagcggggcc

SEQ ID NO: 89 - DNA, 24 bp, Homo sapiens
ccggcgatggggcataaaaccact

SEQ ID NO: 90 - DNA, 23 bp, Homo sapiens
cgcccagcacagcgcacagtagc

SEQ ID NO: 91 - DNA, 20 bp, Homo sapiens
gcccagaatgttcagaccag

SEQ ID NO: 92 - DNA, 20 bp, Homo sapiens
gcaccctcttcacgacaaag

SEQ ID NO: 93 - DNA, 34 bp, Homo sapiens
ccttgtttctctcccatcctcacttcctcaaggc

SEQ ID NO: 94 - DNA, 22 bp, Homo sapiens
caccaccccagcccacagcagc

SEQ ID NO: 95 - DNA, 1002 bp, Homo sapiens
actgcggagatgagggtctagaaggtggtggcggggcatgtggaccgttgtaagggctctggggttcctgggtgggctggcgaagtcctactcacagtgaccaaccatgatgatggtcccgatagaggaggagagggaggaggagggaaaaggaagggtgaggggctcagaggggagagctgggaggaggggagacataggtgggggaaggggtaggagaaaggggaagggagcaagagggtgaggggcaccaggccccatagacgttttggctcagcggccacgaggcttcatcagctcccgccccaaaacggaagcgaggccgtgggggcagcggcagcatggcggggcttgtcttggcggccatggccccgccccctgcccgtccgatcagcgccccgccccgtccccgccccgaccccgccccgggcccgctcaggccccgcccctgccgccggaatcctgaagcccaaggctgcccgggggcggtccggcggcgccggcgatggggcataaaaccactggccacctgccgggctgctcctgcgtgcgctgccgtcccggatccaccgtgcctctgcggcctgcgtgccccgagtccccgcctgtgtcgtctctgtcgccgtccccgtctcctgccaggcgcggagccctgcgagccgcgggtgggccccaggcgcgcagacatgagctgctccgccaaagcgcgctgggctgccggggcgctgggcgtcgcggggctactgtgcgctgtgctgggcgctgtcatgatcgtgatggtgccgtcgctcatcaagcagcaggtccttaaggtgggtgagggagaccccagggggtccgcgcacggacccgggctgttgggcgctgggcgccgggaggacccgcgcgttgcggtgggtgggcgaccgcagcggaatcggcgcccgggcctggcgccgcagaacacgagggaggccaggcgcttcgggaggggctgctgcccgcctccccaccaccctcacc

SEQ ID NO: 96 - DNA, 495 bp, Homo sapiens
catgtcctgcagtgggcaggcagcgggagggacagacttggcgaaggggccgagctcagctttggctgtggggccggaggtgtgcacagacgtccagggcccctggttcccaggcaggcattgcaggcgagtagaagggaaacgtcccatgcagcggggcggggcgtctgacccactggcttcccccacagggagttcaggcacaaaagcaacatcaccttcaacaacaacgacaccgtgtccttcctcgagtaccgcaccttccagttccagccctccaagtcccacggctcggagagcgactacatcatcatgcccaacatcctggtcttggtgaggctgccctgtggcccacgccgcctcgcaccctgacctcgtcccctgtctctcctcccgcctgccccttgtgcagagagcagtccctgaggtggtcggagcgtggggactcacgcctggtgggtggctttcggccctgtgctgtctccaccaccccca

SEQ ID NO: 97 - DNA, 470 bp, Homo sapiens
cctggagggaggaggtccctggcaggctccaacacatgctttagccgggaagcttgaggtggggaaaagctgaggcgggcacagaggaaggtgttgggtggcatctgcgctgtagcccgcagcgtgcggccccagctcatgtgtttgtcattctgtctcctcagagcggggccatggagggggagactcttcacacattctacactcagctggtgttgatgcccaaggtgatgcactatgcccagtacgtcctcctggcgctgggctgcgtcctgctgctggtccctgtcatctgccaaatccggagccaagtaggtgctggccagagggcagcccgggctgacagccattcgcttgcctgctgggggaaaggggcctcagatcggaccctctggccaaccgcagcctggagcccacctccagcagcagtcctgcgtctctgccggagtgggagcggtcactgctggggg

SEQ ID NO: 98 - DNA, 21 bp, Homo sapiens
gcggagcagctcatgtctgcg

SEQ ID NO: 99 - DNA, 31 bp, Homo sapiens
ctttcgcggagcagctcatgtctgcgcgcct

SEQ ID NO: 100 - DNA, 21 bp, Homo sapiens
cgcagacatgagctgctccgc

SEQ ID NO: 101 - DNA, 31 bp, Homo sapiens
aggcgcgcagacatgagctgctccgccaaag

SEQ ID NO: 102 - DNA, 21 bp, Homo sapiens
gcggagcagcgcatgtctgcg

SEQ ID NO: 103 - DNA, 31 bp, Homo sapiens
ctttcgcggagcagcgcatgtctgcgcgcct

SEQ ID NO: 104 - DNA, 21 bp, Homo sapiens
cgcagacatgcgctgctccgc

SEQ ID NO: 105 - DNA, 31 bp, Homo sapiens
aggcgcgcagacatgcgctgctccgccaaag

SEQ ID NO: 106 - DNA, 21 bp, Homo sapiens
ttgggcatgatgatgtagacg

SEQ ID NO: 107 - DNA, 31 bp, Homo sapiens
ggatgttgggcatgatgatgtagacgctctc

SEQ ID NO: 108 - DNA, 21 bp, Homo sapiens
cgactacatcatcatgcccaa

SEQ ID NO: 109 - DNA, 31 bp, Homo sapiens
gagagcgactacatcatcatgcccaacatcc

SEQ ID NO: 110 - DNA, 21 bp, Homo sapiens
ttgggcatgaggatgtagacg

SEQ ID NO: 111 - DNA, 31 bp, Homo sapiens
ggatgttgggcatgaggatgtagacgctctc

SEQ ID NO: 112 - DNA, 21 bp, Homo sapiens
cgactacatcctcatgcccaa

SEQ ID NO: 113 - DNA, 32 bp, Homo sapiens
gagagcgactacatccatcatgcccaacatcc

SEQ ID NO: 114 - DNA, 21 bp, Homo sapiens
tggggccgcacgctgcgggct

SEQ ID NO: 115 - DNA, 31 bp, Homo sapiens
tgagctggggccgcacgctgcgggctacagc

SEQ ID NO: 116 - DNA, 21 bp, Homo sapiens
agcccgcagcgtgcggcccca

SEQ ID NO: 117 - DNA, 31 bp, Homo sapiens
gctgtagcccgcagcgtgcggccccagctca

SEQ ID NO: 118 - DNA, 21 bp, Homo sapiens
tggggccgcaggctgcgggct

SEQ ID NO: 119 - DNA, 31 bp, Homo sapiens
tgagctggggccgcaggctgcgggctacagc

SEQ ID NO: 120 - DNA, 21 bp, Homo sapiens
agcccgcagcctgcggcccca

SEQ ID NO: 121 - DNA, 31 bp, Homo sapiens
gctgtagcccgcagcctgcggccccagctca